(19) United States
(12) Patent Application Publication
(10) Pub. No.: US 2025/0122490 A1
(43) Pub. Date: Apr. 17, 2025

(54) Nucleic Acid-Guided Nucleases

(71) Applicant: GigaMune, Inc., San Francisco, CA (US)

(72) Inventors: David Scott JOHNSON, San Francisco, CA (US); Jan Fredrik SIMONS, San Francisco, CA (US); Yoong Wearn LIM, Pacifica, CA (US); Matthew James SPINDLER, San Francisco, CA (US); Kyle Pierce CARTER, Brisbane, CA (US); Savreet Kaur SANDHU, San Francisco, CA (US); Ellen Kathleen WAGNER, San Francisco, CA (US); Garry COLES, San Mateo, CA (US); Robert EDGAR, Corte Madera, CA (US)

(21) Appl. No.: 18/887,663

(22) Filed: Sep. 17, 2024

Related U.S. Application Data
(63) Continuation of application No. 18/336,922, filed on Jun. 16, 2023, now abandoned.
Application No. 18/336,922 is a continuation of application No. PCT/US2021/065554, filed on Dec. 29, 2021.
(60) Provisional application No. 63/133,089, filed on Dec. 31, 2020.

(51) Int. Cl. C12N 9/22; C12N 15/10; C12N 15/63

(52) U.S. Cl. CPC C12N 9/22; C12N 15/102; C12N 15/63; C07K 2319/09; C12N 2310/20

(57) ABSTRACT

The present disclosure provides novel nucleic acid-guided nucleases and methods of using the nucleases for genome editing. The present disclosure further provides a system for editing a target region in a genome comprising a nucleic acid-guided nuclease, a heterologous guide nucleic acid for complexing with the nucleic acid-guided nuclease, and an editing polynucleotide configured to bind to the target region.

### 1. REFERENCE TO RELATED APPLICATIONS

This application is a continuation of U.S. patent application Ser. No. 18/336,992, filed Jun. 16, 2023, which is a bypass continuation of International Application PCT/US2021/065554, filed Dec. 29, 2021, which claims the benefit of U.S. Provisional Application No. 63/133,089 filed on Dec. 31, 2020, the disclosures of which are hereby incorporated by reference in their entirety.

### 2. SEQUENCE LISTING

The instant application contains a Sequence Listing which has been submitted via Patent Center and is hereby incorporated by reference in its entirety. Said XML copy, created on Jul. 12, 2023, is named 55950US_CRF_sequencelisting.xml and is 2,798,066 bytes in size.

### 3. BACKGROUND

The CRISPR (clustered regularly interspaced short palindromic repeats)-Cas9 system allows targeted alteration of genomic sequences in living cells, making possible ex vivo and in vivo gene editing therapies through targeted nonhomologous end-joining and homology-directed repair. In addition to the canonical Cas9 nuclease family, additional nucleic acid-guided nuclease families have been discovered, including CasX, Cpf1/Cas12a (which includes MAD7), Cas12b, Cas12c, and Cas13.

However, nucleases available in the art have limitations, such as difficulties in purification on a large scale for use in genome engineering or other applications, and challenges in delivery due to their sizes. They have further limitations related to their specificity, processivity, genome editing efficiency, and genome targeting limitations imposed by PAM recognition sequences.

Therefore, there is a need for additional nucleic acid-guided nucleases that provide additional or improved targeting functionality and/or improved function, as compared to enzymes in the Cas9 family. Further, development of various genome editing tools is desired to provide an option to choose an optimal tool for specific application and purposes.

### 4. SUMMARY

The present disclosure provides novel nucleic acid-guided nucleases and methods of using the nucleases for genome editing. The new genome editing tools provided herein are expected to increase flexibility in applying genome editing technologies, because each nuclease has unique characteristics, which can affect target recognition specificity and genetic editing efficiency. Further, the nucleases have desired properties in terms of their genome editing efficiency and specificity. These benefits are important for applications in biomedical research, agriculture, human gene therapy, human cell therapy, and diagnostics, and many other commercial and industrial applications.

Accordingly, one aspect of the present disclosure provides an engineered, non-naturally occurring targetable nuclease system comprising: (a) nucleic acid-guided nuclease, comprising a nuclease polypeptide having at least 95% sequence identity to a sequence selected from SEQ ID NO: 2-273, and (b) at least one engineered guide polynucleotide designed to form a complex with the nuclease and comprising a guide sequence, wherein the guide sequence is designed to hybridize with a target sequence in a eukaryotic cell, and (c) the complex of the nuclease and the guide polynucleotide do not naturally occur.

In some embodiments, the nuclease polypeptide has at least 96%, 97%, 98%, 99%, or 100% sequence identity to a sequence selected from SEQ ID NO: 2-273. In some embodiments, the nuclease polypeptide has less than 100% sequence identity to SEQ ID NO: 2-273. In some embodiments, the nuclease polypeptide has at least 95%, 96%, 97%, 98%, 99%, or 100% sequence identity to a sequence selected from SEQ ID NO: 123, 116, 146, 43, 254, and 175. In some embodiments, the nuclease polypeptide has at least 95%, 96%, 97%, 98%, 99%, or 100% sequence identity to a sequence selected from SEQ ID NO: 123, 146, 254, and 175.

In some embodiments, the nuclease polypeptide comprises a sequence selected from SEQ ID NO: 815-822. In some embodiments, the nuclease polypeptide comprises sequences of SEQ ID NO: 815-822

In some embodiments, the nuclease polypeptide has at least 95%, 96%, 97%, 98%, 99%, or 100% sequence identity to a sequence selected from SEQ ID NO: 116 and 43.

In some embodiments, the nuclease polypeptide comprises a sequence of SEQ ID NO: 123. In some embodiments, the nuclease polypeptide comprises a sequence of SEQ ID NO: 116. In some embodiments, the nuclease polypeptide comprises a sequence of SEQ ID NO: 146. In some embodiments, the nuclease polypeptide comprises a sequence of SEQ ID NO: 32. In some embodiments, the nuclease polypeptide comprises a sequence of SEQ ID NO: 254. In some embodiments, the nuclease polypeptide comprises a sequence of SEQ ID NO: 175.

In some embodiments, the nuclease polypeptide is fused to a fusion peptide. In some embodiments, the fusion peptide is a signal peptide fused in-frame to the nuclease polypeptide. In some embodiments, the fusion peptide is a nuclear localization sequence fused to the nuclease polypeptide. In some embodiments, the nuclear localization sequence has a sequence selected from SEQ ID NO: 628-631.

In some embodiments, the nuclease polypeptide is originated from Acidaminococcus massiliensis, Acidaminococcus sp., Acinetobacter indicus, Agathobacter rectalis, Anaerovibrio lipolyticus, Bacteroidales bacterium, Bacteroides galacturonicus, Bacteroides plebeius, Bacteroidetes bacterium, Butyrivibrio fibrisolvens, Butyrivibrio hungatei, Butyrivibrio sp., Candidatus Falkowbacteria bacterium, Candidatus Falkowbacteria bacterium, Candidatus Gottesmanbacteria bacterium, Candidatus Jacksonbacteria bacterium, Candidatus Magasanikbacteria bacterium, Candidatus Moranbacteria bacterium, Candidatus Pacebacteria bacterium, Candidatus Roizmanbacteria bacterium, Candidatus Ryanbacteria bacterium, Candidatus Saccharibacteria bacterium, Candidatus Sungbacteria bacterium, Candidatus Uhrbacteria bacterium, Candidatus Wildermuthbacteria bacterium, Candidatus Yonathbacteria bacterium, Catenovulum sp., Clostridiales bacterium, Clostridium sp., Coprococcus eutactus, Coprococcus sp., Deltaproteobacteria bacterium, Elizabethkingia sp., Eubacteriaceae bacterium, Eubacterium eligens, Eubacterium rectale, Eubacterium sp., Eubacterium ventriosum, Fibrobacter sp., Fibrobacter succinogenes, Firmicutes bacterium, Flavobacterium branchiophilum, Francisella hispaniensis, Francisella novicida, Francisella philomiragia, Francisella tularensis, Lachnospiraceae bacterium, Lachnospira pectinoschiza, Lentisphaeria bacterium, Leptospiraceae bacterium, Leptospira sp., Moraxella bovis, Moraxella bovoculi, Moraxella lacunata, Moraxella ovis, Moraxella sp., Muribaculaceae bacterium, Patescibacteria group bacterium, Phycisphaerae bacterium, Phycisphaerales bacterium, Porphyromonadaceae bacterium, Porphyromonas crevioricanis, Prevotella brevis, Prevotellaceae bacterium, Prevotella copri, Prevotellamassilia sp., Prevotella ruminicola, Prevotella sp., Prolixibacteraceae bacterium, Pseudobutyrivibrio sp., Pseudobutyrivibrio xylanivorans, Psychrobacter sp., Ruminococcaceae bacterium, Ruminococcus sp., Sedimentisphaera cyanobacteriorum, Sneathia amnii, Spirochaetia bacterium, Succinivibrionaceae bacterium, or Treponema sp.

In another aspect, the present disclosure provides a polynucleotide comprising a first polynucleotide segment encoding the nucleic acid-guided nuclease having at least 95% sequence identity to a sequence selected from SEQ ID NO: 2-273.

In some embodiments, the polynucleotide further comprises a second polynucleotide segment encoding a fusion peptide.

In some embodiments, the first polynucleotide segment has been codon optimized for expression in mammalian cells. In some embodiments, the first polynucleotide segment has been codon optimized for expression in human cells.

In some embodiments, the first polynucleotide segment has a sequence having at least 95%, 96%, 97%, 98%, or 99% sequence identity to a sequence selected from SEQ ID NO: 722-766. In some embodiments, the first polynucleotide segment has a sequence selected from SEQ ID NO: 722-766. In some embodiments, the polynucleotide further comprises the sequence selected from SEQ ID NO: 767-811.

In some embodiments, the first polynucleotide segment has been codon optimized for expression in bacterial cells. In some embodiments, the polynucleotide comprises the sequence selected from SEQ ID NO: 632-676.

In some embodiments, the first polynucleotide segment has a sequence selected from SEQ ID NO: 677-721.

In yet another aspect, the present disclosure provides a vector encoding the nucleic acid-guided nuclease, comprising the polynucleotide of any one of claims 20-29.

In some embodiments, the vector further comprises a promoter operably linked to the polynucleotide encoding the nucleic acid-guided nuclease.

In one aspect, the present disclosure provides a host cell comprising the polynucleotide provided herein or the vector provided herein.

One aspect of the present disclosure provides a method of generating a nucleic acid-guided nuclease comprising the steps of: culturing the host cell described herein, and isolating the nucleic acid-guided nuclease from the host cell culture.

In one aspect, the present disclosure provides a method of modifying a target region of a eukaryotic or prokaryotic genome, comprising the steps of: contacting a sample comprising the target region with a nucleic acid-guided nuclease having at least 90% sequence identity to a sequence selected from the group consisting of SEQ ID NO: 2-273, and a guide nucleic acid complexed with the nucleic acid-guided nuclease, and allowing the nucleic acid-guided nuclease to modify the target region.

In some embodiments, the contacting step is performed further in the presence of a homology template configured to bind to the target region.

In some embodiments, the guide nucleic acid is a heterologous guide nucleic acid.

In some embodiments, the nucleic acid-guided nuclease is originated from Acidaminococcus massiliensis, Acidaminococcus sp., Acinetobacter indicus, Agathobacter rectalis, Anaerovibrio lipolyticus, Bacteroidales bacterium, Bacteroides galacturonicus, Bacteroides plebeius, Bacteroidetes bacterium, Butyrivibrio fibrisolvens, Butyrivibrio hungatei, Butyrivibrio sp., Candidatus Falkowbacteria bacterium, Candidatus Falkowbacteria bacterium, Candidatus Gottesmanbacteria bacterium, Candidatus Jacksonbacteria bacterium, Candidatus Magasanikbacteria bacterium, Candidatus Moranbacteria bacterium, Candidatus Pacebacteria bacterium, Candidatus Roizmanbacteria bacterium, Candidatus Ryanbacteria bacterium, Candidatus Saccharibacteria bacterium, Candidatus Sungbacteria bacterium, Candidatus Uhrbacteria bacterium, Candidatus Wildermuthbacteria bacterium, Candidatus Yonathbacteria bacterium, Catenovulum sp., Clostridiales bacterium, Clostridium sp., Coprococcus eutactus, Coprococcus sp., Deltaproteobacteria bacterium, Elizabethkingia sp., Eubacteriaceae bacterium, Eubacterium eligens, Eubacterium rectale, Eubacterium sp., Eubacterium ventriosum, Fibrobacter sp., Fibrobacter succinogenes, Firmicutes bacterium, Flavobacterium branchiophilum, Francisella hispaniensis, Francisella novicida, Francisella philomiragia, Francisella tularensis, Lachnospiraceae bacterium, Lachnospira pectinoschiza, Lentisphaeria bacterium, Leptospiraceae bacterium, Leptospira sp., Moraxella bovis, Moraxella bovoculi, Moraxella lacunata, Moraxella ovis, Moraxella sp., Muribaculaceae bacterium, Patescibacteria group bacterium, Phycisphaerae bacterium, Phycisphaerales bacterium, Porphyromonadaceae bacterium, Porphyromonas crevioricanis, Prevotella brevis, Prevotellaceae bacterium, Prevotella copri, Prevotellamassilia sp., Prevotella ruminicola, Prevotella sp., Prolixibacteraceae bacterium, Pseudobutyrivibrio sp., Pseudobutyrivibrio xylanivorans, Psychrobacter sp., Ruminococcaceae bacterium, Ruminococcus sp., Sedimentisphaera cyanobacteriorum, Sneathia amnii, Spirochaetia bacterium, Succinivibrionaceae bacterium, or Treponema sp.

In some embodiments, the nucleic acid-guided nuclease has at least 95%, 96%, 97%, 98%, 99% or 100% identity to a sequence selected from SEQ ID NO: 2-273. In some embodiments, the nuclease polypeptide has at least 95%, 96%, 97%, 98%, 99%, or 100% sequence identity to a sequence selected from SEQ ID NO: 123, 116, 146, 43, 254, and 175. In some embodiments, the nuclease polypeptide has at least 95%, 96%, 97%, 98%, 99%, or 100% sequence identity to a sequence selected from SEQ ID NO: 123, 146, 254, and 175.

In some embodiments, the nuclease polypeptide comprises a sequence selected from SEQ ID NO: 815-822. In some embodiments, the nuclease polypeptide comprises sequences of SEQ ID NO: 815-822.

In some embodiments, the nuclease polypeptide has at least 95%, 96%, 97%, 98%, 99%, or 100% sequence identity to a sequence selected from SEQ ID NO: 116 and 43. In some embodiments, the nuclease polypeptide comprises a sequence selected from 123, 116, 146, 32, 254, 275, and 175.

In some embodiments, the sample comprises a eukaryotic cell. In some embodiments, the sample comprises a bacterial cell. In some embodiments, the sample comprises a plant cell. In some embodiments, the sample comprises a mammalian cell. In some embodiments, the sample comprises an immune cell. In some embodiments, the immune cell is a B cell or T cell.

In some embodiments, a T cell receptor is engineered into the genome. In some embodiments, an endogenous T cell receptor is disrupted. In some embodiments, a T cell receptor is engineered into the genome and an endogenous T cell receptor is disrupted.

In some embodiments, the homology template includes a sequence complementary to the target region. In some embodiments, the homology template includes an insertion, deletion, or modification compared to the target region.

In some embodiments, the guide nucleic acid is an engineered, non-naturally occurring polynucleotide. In some embodiments, the guide nucleic acid and the homology template form a single polynucleotide.

In another aspect, the present disclosure provides a cell, tissue or organism comprising a genome modified by the method of the present disclosure.

### 5. BRIEF DESCRIPTION OF THE DRAWINGS

FIG. 1. Histogram showing amino acid percent identity to MAD7 for novel Cas enzymes (SEQ IDS 2-273), which we refer to as the “GIG-” nucleases or “GIG-” enzymes, identified in a sequence search of 134,655 prokaryotic genomes in the NCBI Genbank database.

FIG. 2. Sequence tree showing the relationship among the novel GIG-Cas enzymes (SEQ IDS 2-273) identified in a sequence search of 134,655 prokaryotic genomes in the NCBI Genbank database.

FIG. 3. Sequence logo summarizing crRNA CRISPR repeats (SEQ IDS 274-627) in the genomic vicinity of the novel GIG-Cas enzymes (SEQ IDS 2-273) identified in a sequence search of 134,655 prokaryotic genomes in the NCBI Genbank database.

FIG. 4. Vector map of T7p14 DNA construct for in vitro transcription and translation (SEQ ID NO: 814).

FIGS. 5A-5C. Functional assessment of novel GIG-Cas enzymes through an in vitro GFP reporter assay. FIG. 5A: GIG-1 (SEQ ID NO: 123) and GIG-2 (SEQ ID NO: 43); FIG. 5B: GIG-4 (SEQ ID NO: 254) and GIG-5 (SEQ ID NO: 28); FIG. 5C: GIG-3 (SEQ ID NO: 79). Abscissa: incubation time, each cycle corresponds to 10 min for a total of 18 h Ordinate: GFP relative fluorescence signal (excitation/emission: 485/520 nm, resp).

FIG. 6. Heatmap of PAM activities of the GIG-Cas enzymes, identified using the in vitro screening system of Maxwell et al. (Methods, 2018).

FIGS. 7A-7D. PAM sequence motifs that function with novel GIG or other Cas enzymes, identified using the in vitro screening system of Maxwell et al. (Methods, 2018).

FIG. 8. Vector map of pET21 construct for bacterial expression (SEQ ID NO: 812).

FIG. 9. SDS-PAGE analysis of purified recombinant GIG nucleases (GIG-1, GIG-2, GIG-5, GIG-10, GIG-12, GIG-15, GIG-16, and GIG-17). 1 μg of each protein was loaded on 4-20% gel. (H) samples were purified by His-purification; (C) samples were CEX purified following His-purification.

FIGS. 10A-10C. SE-HPLC analysis of purified GIG-Cas nucleases (GIG-1, GIG-2, GIG-5, GIG-10, GIG-12, GIG-15, GIG-16 and GIG-17) following His and CEX purification. FIG. 10A: AsCas12a, MAD7, GIG-1 and GIG-2; FIG. 10B: GIG-5, GIG-10, GIG-12 and GIG-15; FIG. 10C: GIG-16 and GIG-17.

FIG. 11. Knockdown and HDR Efficiency of selected GIG nucleases at the human TRAC locus in Jurkat cells. Cells were electroporated with the RNPs consisting of the indicated nuclease and TRAC-targeting sgRNA (GR-31, GR-40, and GR-42). As a negative control, RNP consisting of AsCas12a and a scrambled sgRNA was also electroporated. Additionally, each sample was also electroporated with a homology-directed repair (HDR) template for GFP expression. Cells were stained with fluorescently-conjugated antibodies for CD3 and TCRaβ and analyzed by flow cytometry 5 days after electroporation. Higher knockdown efficiency indicates lower expression levels of CD3 and TCRaβ. Cells that successfully incorporated the HDR template express GFP.

FIG. 12. Knockdown and HDR Efficiency of selected GIG nucleases at the human TRAC locus in Jurkat cells. Cells were electroporated with RNPs consisting of the indicated nuclease and TRAC-targeting sgRNA, as well as an HDR template for GFP expression. As a negative control, RNPs were electroporated without the HDR template. Cells were stained with fluorescently-conjugated antibodies for CD3 and TCRaβ and analyzed by flow cytometry 5 days after electroporation. Higher knockdown efficiency indicates lower expression levels of CD3 and TCRaβ. Cells that successfully incorporated the HDR template express GFP.

FIG. 13. Knockdown efficiency of AsCas12a and GIG-17 nucleases at the human B2M locus in Jurkat cells. Cells were electroporated with RNPs consisting of the indicated nuclease and three unique B2M-targeting sgRNAs (GR-44, GR-45, GR-46). Cells were stained with fluorescently-conjugated antibody for HLA-A, B, C and analyzed by flow cytometry 5 days after electroporation. Higher knockdown efficiency indicates higher levels of B2M deficient cells.

FIG. 14. Knockdown efficiency of AsCas12a and GIG-17 nucleases at the human HLA-A*02:01 locus in T2 cells. Cells were electroporated with RNPs consisting of the indicated nuclease and three unique HLA-A*02:01-targeting sgRNAs (GR-71, GR-72 or GR-73). Cells were stained with a fluorescently-conjugated antibody for HLA-A2 and analyzed by flow cytometry 5 days after electroporation. Higher knockdown efficiency indicates higher levels of HLA-A2 deficient cells.

FIG. 15. Vector map of pReceiver lentiviral construct for mammalian expression (SEQ ID 813).

The figures depict various embodiments of the present invention for purposes of illustration only. One skilled in the art will readily recognize from the following discussion that alternative embodiments of the structures and methods illustrated herein can be employed without departing from the principles of the invention described herein.

### 6. DETAILED DESCRIPTION

### 6.1. Definitions

Unless defined otherwise, all technical and scientific terms used herein have the meaning commonly understood by one of ordinary skill in the art to which the invention pertains. As used herein, the following terms have the meanings ascribed to them below.

The term “heterologous guide nucleic acid” as used herein refers to a guide nucleic acid that is capable of complexing with a nucleic acid-guided nuclease to form a ribonucleic acid particle (RNP), wherein the RNP does not exist in nature.

The term “compatible” as used herein refers to a guide nucleic acid and nucleic-acid guided nuclease that are capable of complexing to form an RNP that functions as a targeted nuclease complex.

The terms “variant” or “mutant” as used herein refers to a biological material (e.g., protein, polynucleotide, etc.) exhibiting qualities that deviates from what occurs in nature. For example, a variant or mutant can be a polypeptide having a mutation from a wild type polypeptide at one or more amino acids, or which contains addition, deletion or substitution of one or more amino acids.

The terms “crRNA”, “gRNA” and “guide RNA” are used interchangeably as described elsewhere, e.g., PCT/US2013/074667. In general, gRNA is a polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence is about or more than 50%, 60%, 70%, 80%, 90%, 95%, 99%, or more.

The practice of the present invention employs, unless otherwise indicated, conventional techniques of immunology, biochemistry, chemistry, molecular biology, microbiology, cell biology, genomics, recombinant DNA, which are well known for those will skill in the art. See Green and Sambrook (Molecular Cloning: A Laboratory Manual), Current Protocols in Molecular Biology (Ausubel, et al., eds.), Antibodies: a Laboratory Manual (Harlow & Taylor, eds.).

### 6.2. Other interpretational conventions

Ranges recited herein are understood to be shorthand for all of the values within the range, inclusive of the recited endpoints. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50.

### 6.3. Nucleic Acid-Guided Nucleases

In a first aspect, a nucleic acid-guided nuclease is provided. The nucleases are functional in prokaryotic and eukaryotic cells and are useful for in vitro, ex vivo, and in vivo genome editing applications. In some embodiments, the nucleic acid-guided nucleases are naturally occurring. In some embodiments, the nucleic acid-guided nucleases are non-naturally occurring. In certain embodiments, the non-naturally occurring nuclease is an engineered nuclease. In some embodiments, the nucleic acid-guided nucleases are purified proteins.

In some embodiments, nucleic acid guided nucleases are part of a “targetable nuclease system” comprising a nucleic acid guided nuclease and a guide nucleic acid. A targetable nuclease system can be used to bind, cleave, modify, and/or edit a target polynucleotide sequence, often referred to as a “target sequence”. Methods, systems, vectors, polynucleotides, and compositions described herein may be used in various applications including altering or modifying synthesis of a gene product, such as a protein, polynucleotide cleavage, polynucleotide editing, polynucleotide splicing, trafficking of target polynucleotide, isolation of target polynucleotide, visualization of target polynucleotide, etc. Aspects of the current invention also include methods and uses of the compositions and systems described herein in “genome engineering”, defined as altering or manipulating the expression of one or more gene products in prokaryotic, archaeal, or eukaryotic cells in vitro, in vivo, or ex vivo. For example, use of nucleic acid guided nucleases are described in U.S. Pat. No. 10,011,849, incorporated by reference in its entirety herein.

### 6.3.1. Nucleases

The present disclosure provides novel naturally and non-naturally occurring nucleic acid-guided nucleases. In some embodiments, suitable nucleic acid-guided nucleases are obtained from an organism from a genus which includes but is not limited to: Moraxella, Acidaminococcus, Francisella, Lachnospira, Butyrivibrio, Clostridium, Coprococcus, Prevotella, Flavobacterium, Eubacterium, Sedimentisphaera, Limihaloglobus, Pseudobutyrivibrio, Anaerovibrio, Psychrobacter, Acinetobacter, Catenovulum, Bacteroides, Ruminococcus, Porphyromonas, Elizabethkingia, and Prevotellamassilia. In some embodiments, the nucleic-acid guided nucleases are a variant or a modification of a naturally occurring nuclease.

In some embodiments, the novel nucleases comprise less than 95%, 90%, 80%, 70%, 60%, 50%, 40%, 30%, or 20% sequence identity to any previously disclosed Cpf1, Cas12a, and MAD7 enzymes. Further, nucleases provided herein are different from the Cpf1, Cas12a, and MAD7 enzymes known in the art. For example, endonucleases of the present disclosure have a sequence different from the sequences disclosed in U.S. Pat. No. 9,790,490B2. In some embodiments, the novel nuclease of the present disclosure comprises less than 95%, 90%, 80%, 70%, 60%, 50% or 40% sequence identity to any of the sequences disclosed in U.S. Pat. No. 9,790,490B2. U.S. Pat. No. 9,790,490B2 and sequences disclosed therein are incorporated by reference in their entireties herein.

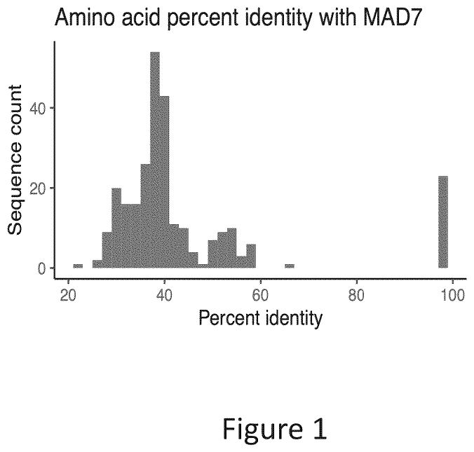

The term “orthologue” or “homologue” as used herein refers to a protein having a sequence having at least 80%, or preferably at least 85%, sequence identity, when aligned with a suitable sequence alignment algorithm. On average, the novel nucleases reported herein has only about 38% sequence identity to previously reported Cpf1 sequences of subtype V-A (see U.S. Pat. No. 9,790,490B2) (FIG. 1). So, most nucleases reported in the present disclosures do not have a previously known homologue.

In some embodiments, the nuclease is obtained from a bacterial genomic locus for a gene selected from the families cas1, cas2, and cpf1 and a CRISPR array. In some embodiments, Cpf1 or Cpf1-like peptide sequences are originated from organisms of the genera Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Glucomacetobacter, Neiserria, Roseburia, Parvibaculum, Staphylococcus, Nitratifractor, Mycoplasma, or Campylobacter. In some embodiments, Cpf1 or Cpf1-like peptide sequences are originated from organisms other than the genera Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Glucomacetobacter, Neiserria, Roseburia, Parvibaculum, Staphylococcus, Nitratifractor, Mycoplasma, or Campylobacter.

In some embodiments, the nucleic acid-guided nuclease of the present disclosure comprises a nuclease polypeptide. The nuclease polypeptide is a polypeptide having a sequence selected from SEQ ID NO: 2-273. In some embodiments, the nuclease polypeptide is a polypeptide having less than 100% sequence identity to a sequence selected from SEQ ID NO: 2-273. In some embodiments, the nuclease polypeptide has at least 96%, 97%, 98%, or 99% sequence identity to a sequence selected from SEQ ID NO: 2-273. In some embodiments, the nuclease polypeptide has at least 95%, 96%, 97%, 98%, 99%, or 100% sequence identity to a sequence selected from SEQ ID NO: 123, 116, 146, 43, 254, and 175.

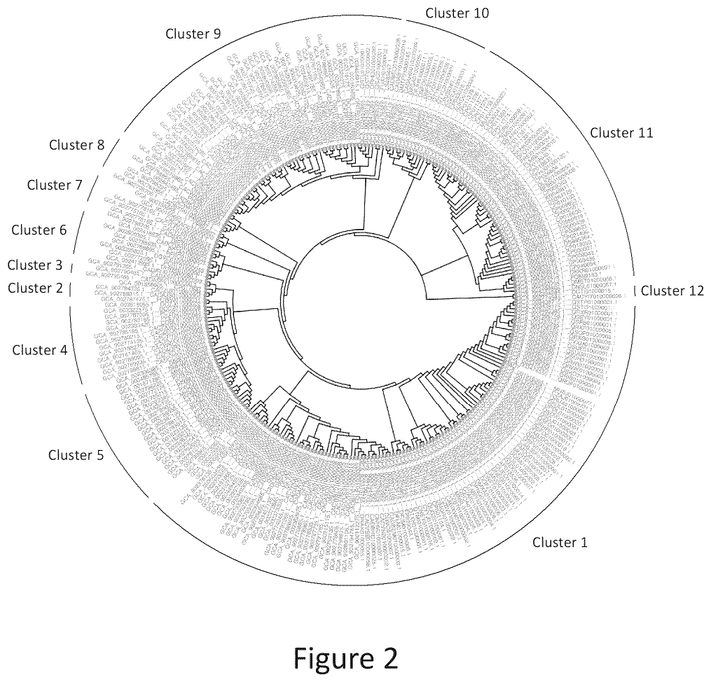

In some embodiments, the nuclease polypeptide is in cluster 1 described in Example 1 and FIG. 2. In some embodiments, the nuclease polypeptide comprises a sequence selected from SEQ ID Nos: 188, 204, 221, 256, 240, 233, 189, 202, 185, 247, 191, 201, 246, 81, 83, 243, 88, 258, 223, 131, 214, 226, 85, 231, 79, 80, 217, 238, 87, 254, 248, 241, 242, 65, 94, 95, 143, 176, 17, 169, 165, 160, 172, 157, 166, 163, 10, 16, 122, 126, 139, 144, 145, 23, 155, 123, 137, 138, 18, 48, 125, 127, 128, 135, 136, 150, 153, 1, 59, 15, 134, 171, 32, 175, 184, 159, 156, 199, 147, 146, 149, 154, 148, 198, 60, 120, 19, 197, 161, 173, 174, 50, 49, 196, 5, 130, 3, 200, 74, 97, 177, 33, 41, and 86.

In some embodiments, the nuclease polypeptide is in cluster 2 described in Example 1 and FIG. 2. In some embodiments, the nuclease polypeptide comprises a sequence selected from SEQ ID Nos: 228, 236, and 8.

In some embodiments, the nuclease polypeptide is in cluster 3 described in Example 1 and FIG. 2. In some embodiments, the nuclease polypeptide comprises a sequence selected from SEQ ID Nos: 245 and 272.

In some embodiments, the nuclease polypeptide is in cluster 4 described in Example 1 and FIG. 2. In some embodiments, the nuclease polypeptide comprises a sequence selected from SEQ ID Nos: 101, 102, 69, 212, 255, 237, 207, 216, 235, 227, 229, 70, 105, and 170.

In some embodiments, the nuclease polypeptide is in cluster 5 described in Example 1 and FIG. 2. In some embodiments, the nuclease polypeptide comprises a sequence selected from SEQ ID Nos: 110, 113, 111, 73, 66, 54, 55, 112, 75, 106, 109, 108, 53, 118, 100, 103, 114, 56, 67, and 162.

In some embodiments, the nuclease polypeptide is in cluster 6 described in Example 1 and FIG. 2. In some embodiments, the nuclease polypeptide comprises a sequence selected from SEQ ID Nos: 104, 107, 260, 253, 91, 99, 92, 262, and 271.

In some embodiments, the nuclease polypeptide is in cluster 7 described in Example 1 and FIG. 2. In some embodiments, the nuclease polypeptide comprises a sequence selected from SEQ ID Nos: 269, 220, 225, 266, and 186.

In some embodiments, the nuclease polypeptide is in cluster 8 described in Example 1 and FIG. 2. In some embodiments, the nuclease polypeptide comprises a sequence selected from SEQ ID Nos: 194, 203, 115, 211, 273, and 249.

In some embodiments, the nuclease polypeptide is in cluster 9 described in Example 1 and FIG. 2. In some embodiments, the nuclease polypeptide comprises a sequence selected from SEQ ID Nos: 132, 133, 124, 152, 151, 72, 206, 24, 25, 68, 195, 232, 30, 12, 182, 252, 259, 222, 251, 190, 209, 239, 250, 192, 205, 71, 76, 215, 93, 264, 208, 267, 183, 265, 193, 210, 89, 263, 268, 270, 213, 224, 218, 257, 36, 178, 187, and 244.

In some embodiments, the nuclease polypeptide is in cluster 10 described in Example 1 and FIG. 2. In some embodiments, the nuclease polypeptide comprises a sequence selected from SEQ ID Nos: 158, 230, 234, 140, 164, 142, 141, 180, 77, 78, 167, 13, 35, and 179.

In some embodiments, the nuclease polypeptide is in cluster 11 described in Example 1 and FIG. 2. In some embodiments, the nuclease polypeptide comprises a sequence selected from SEQ ID Nos: 62, 121, 61, 82, 4, 29, 39, 117, 58, 57, 40, 27, 7, 6, 31, 9, 28, 38, 37, 26, 34, 129, 96, 181, 168, 47, 261, 2, 46, 22, 63, 42, 44, 43, 45, 20, 51, 52, 64, 11, 84, 116, 21, 14, and 119.

In some embodiments, the nuclease polypeptide is in cluster 12 described in Example 1 and FIG. 2. In some embodiments, the nuclease polypeptide comprises a sequence selected from SEQ ID Nos: 219 and 90.

In some embodiments, the nuclease polypeptide is in cluster 3, 4, 5, 6, 7, 8, 9, or 10 described in Example 1 and FIG. 2.

In some embodiments, the nuclease polypeptide is not in cluster 1 described in Example 1 and FIG. 2. In some embodiments, the nuclease polypeptide is not in cluster 2 described in Example 1 and FIG. 2. In some embodiments, the nuclease polypeptide is not in cluster 3, 4, 5, 6, 7, 8, 9, or 10 described in Example 1 and FIG. 2. In some embodiments, the nuclease polypeptide is not in cluster 11 described in Example 1 and FIG. 2.

In some embodiments, the nuclease polypeptide comprises a conserved peptide sequence identified through a multiple sequence alignment of nucleases which are putatively evolutionarily related. In some embodiments, the nuclease polypeptide comprises one or more of the conserved peptide sequences of cluster 1 (SEQ ID NO: 815-822). In some embodiments, the nuclease polypeptide comprises one or more of the conserved peptide sequences of cluster 4 (SEQ ID NO: 823-832). In some embodiments, the nuclease polypeptide comprises the conserved peptide sequence of cluster 6 (SEQ ID NO: 833). In some embodiments, the nuclease polypeptide comprises the conserved peptide sequence of cluster 7 (SEQ ID NO: 834). In some embodiments, the nuclease polypeptide comprises one or more of the conserved peptide sequences of cluster 9 (SEQ ID NO: 835-840). In some embodiments, the nuclease polypeptide conserved peptide one or more of the consensus sequences of cluster 10 (SEQ ID NO: 841-844).

In some embodiments, the nuclease polypeptide comprises all the consensus sequences of cluster 1 (SEQ ID NO: 815-822). In some embodiments, the nuclease polypeptide comprises all the consensus sequences of cluster 4 (SEQ ID NO: 823-832). In some embodiments, the nuclease polypeptide comprises all the consensus sequences of cluster 9 (SEQ ID NO: 835-840). In some embodiments, the nuclease polypeptide comprises all the consensus sequences of cluster 10 (SEQ ID NO: 841-844).

In some embodiments, the nuclease polypeptide has at least 96%, 97%, 98%, or 99% sequence identity to SEQ ID NO: 123. In some embodiments, the nuclease polypeptide comprises a sequence of SEQ ID NO: 123.

In some embodiments, the nuclease polypeptide has at least 96%, 97%, 98%, or 99% sequence identity to SEQ ID NO: 116. In some embodiments, the nuclease polypeptide comprises a sequence of SEQ ID NO: 116.

In some embodiments, the nuclease polypeptide has at least 96%, 97%, 98%, or 99% sequence identity to SEQ ID NO: 146. In some embodiments, the nuclease polypeptide comprises a sequence of SEQ ID NO: 146.

In some embodiments, the nuclease polypeptide has at least 96%, 97%, 98%, or 99% sequence identity to SEQ ID NO: 32. In some embodiments, the nuclease polypeptide comprises a sequence of SEQ ID NO: 32.

In some embodiments, the nuclease polypeptide has at least 96%, 97%, 98%, or 99% sequence identity to SEQ ID NO: 254. In some embodiments, the nuclease polypeptide comprises a sequence of SEQ ID NO: 254.

In some embodiments, the nuclease polypeptide has at least 96%, 97%, 98%, or 99% sequence identity to SEQ ID NO: 175. In some embodiments, the nuclease polypeptide comprises a sequence of SEQ ID NO: 175.

In some embodiments, the polypeptides are engineered from the native sequence for specific functional properties. In some embodiments, these engineered polypeptides have 99%, 95%, 90%, 75%, or 50% sequence identity to the native sequence.

In some embodiments, the nuclease polypeptide is a polypeptide having at least 99% sequence identity to a sequence selected from SEQ ID NO: 1-273. In some embodiments, the nuclease polypeptide is a polypeptide having at least 98% sequence identity to a sequence selected from SEQ ID NO: 1-273. In some embodiments, the nuclease polypeptide is a polypeptide having at least 97% sequence identity to a sequence selected from SEQ ID NO: 1-273. In some embodiments, the nuclease polypeptide is a polypeptide having at least 96% sequence identity to a sequence selected from SEQ ID NO: 1-273. In some embodiments, the nuclease polypeptide is a polypeptide having at least 95% sequence identity to a sequence selected from SEQ ID NO: 1-273. In some embodiments, the nuclease polypeptide is a polypeptide having at least 94% sequence identity to a sequence selected from SEQ ID NO: 1-273. In some embodiments, the nuclease polypeptide is a polypeptide having at least 93% sequence identity to a sequence selected from SEQ ID NO: 1-273. In some embodiments, the nuclease polypeptide is a polypeptide having at least 92% sequence identity to a sequence selected from SEQ ID NO: 1-273. In some embodiments, the nuclease polypeptide is a polypeptide having at least 91% sequence identity to a sequence selected from SEQ ID NO: 1-273. In some embodiments, the nuclease polypeptide is a polypeptide having at least 90% sequence identity to a sequence selected from SEQ ID NO: 1-273.

In some embodiments, the nucleic acid-guided nuclease is a recombinant protein. In some embodiments, the nucleic acid-guided nuclease is expressed from a codon-optimized polynucleotide. In some embodiments, the nucleic acid-guided nuclease is expressed from a cell culture.

### 6.3.2. Engineered Nucleases

In some embodiments, an engineered nucleic acid-guided nuclease is used. In some embodiments, the engineered nucleic acid-guided nuclease is chemically or biologically modified. In some embodiments, the engineered nucleic acid-guided nuclease is modified to increase expression from a host cell, optimize for human or mammalian codons (See PCT/US2013/074667 incorporated by reference), increase stability of the protein, increase its gene editing efficiency, reduce off-target specificity, or change PAM sequence specificity. In some embodiments, the engineered nucleic-acid guided nuclease is modified for desired targeting in vivo or in vitro.

In some embodiments, one or more modifications previously described to associated with changes in the nucleic acid-guided nuclease functions are introduced to the nucleases described herein. In some embodiments, one or more mutations or modifications are made in a catalytic domain. In some embodiments, the catalytic activity of the nuclease is reduced or destroyed so that the DNA-binding activity is retained but the enzymatic function of the nuclease is reduced or destroyed. In some embodiments the inactivated nuclease is fused to one or more functional domains, for example, functional domains having methylase activity, demethylase activity, transcription activation activity, transcription repression activity, transcription release factor activity, histone modification activity, RNA cleavage activity, DNA cleavage activity, nucleic acid binding activity, exonuclease activity, single base-editing activity, recombinase activity, integrase activity, reverse transcription activity, or molecular switches. A “functional variant” of a protein herein refers to a variant of such protein which retains at least partial activity of a protein. In some embodiments, engineered nucleases of the current application comprise functional variants of naturally occurring nucleases disclosed in the current application. Functional variants are not always homologues.

In some embodiments, to improve or reduce specificity, the primary residues for mutagenesis are in the RuvC domain of the nuclease, see e.g., Slaymaker et al., 2015, “Rationally engineered Cas9 nucleases with improved specificity” incorporated by reference in its entirety herein. In some embodiments, mutants are designed to accommodate modifications in PAM recognition, for example by choosing mutations that alter PAM specificity and combining those mutations with nt-groove mutations that increase (or decrease) specificity for on-target vs. off-target sequences. In some embodiments, PAM recognition sites of the nucleases described herein can be substituted with a PAM recognition site of a different nuclease to change its PAM specificity.

In some embodiments, mutations are made specifically to the REC lob, REC1 domain, REC2 domain, Nuc lobe, PAM-interacting domain, WED domain, and/or the bridge helix (BH), see e.g., Paul & Montoya, Biomedical Journal, 43 (1): 8-17.

In some embodiments, mutations comprise modification of amino acids that are positively or negatively charged, hydrophobic or hydrophilic, located in a structural groove or other structural component of the nuclease, substitute any residue with an alanine residue, or are polar or nonpolar.

In some embodiments, engineered nucleases are fusions of any number of the enzymes listed herein or fusions between the enzymes listed herein and any other Cas enzyme. Engineered nucleases can comprise 99%, 95%, 90%, 80%, 70%, 60%, 50%, 40%, or 30% sequence identity with any of the enzymes listed herein. In some embodiments, the engineered nuclease comprises a fragment of a naturally occurring nuclease described herein. In some embodiments, a fusion is made by substituting one or more functional domains of a nuclease described herein.

In some embodiments, engineered nucleases are generated by modifying non-conserved sequences in Table 2. In certain embodiments, any nuclease from Cluster 1 (Table 2) is mutated at one or more amino acid positions outside of amino acids 630-652, 891-901, 915-931, 1034-1054, 1058-1063, 1217-1229, 1299-1307, 1308-1335, and 1588-1589. In some embodiments, the engineered nuclease comprises conserved sequences of Cluster 1 (SEQ ID Nos: 815-822).

In certain embodiments, any nuclease from Cluster 4 is modified at one or more amino acid positions outside of amino acids 92-99, 106-111, 113-152, 223-239, 291-303, 396-404, 409-421, 731-791, or 816-874. In some embodiments, the engineered nuclease comprises conserved sequences of Cluster 4 (SEQ ID Nos: 823-832).

In certain embodiments, any nuclease in Cluster 6 is mutated outside of amino acid positions comprising amino acid positions 1120-1126. In some embodiments, the engineered nuclease comprises the conserved sequence of Cluster 6 (SEQ ID NO: 833).

In certain embodiments, any nuclease from Cluster 7 is mutated in one or more amino acid positions outside of amino acids 600-654. In some embodiments, the engineered nuclease comprises the conserved sequence of Cluster 7 (SEQ ID NO: 834).

In certain embodiments, any nuclease from Cluster 9 is mutated outside of amino acid positions comprising amino acids 492-501, 596-625, 685-695, 697-707, 841-891, or 1191-1227. In some embodiments, the engineered nuclease comprises the conserved sequences of Cluster 9 (SEQ ID NO: 835-840).

In certain embodiments, any nuclease from Cluster 10 is mutated in one or more amino acid positions outside of amino acids 159-215, 630-655, 868-879, or 1052-1076. In some embodiments, the engineered nuclease comprises the conserved sequences of Cluster 10 (SEQ ID Nos: 841-844).

In certain embodiments of the invention, the engineered nuclease is a fusion protein comprising conserved sequences present in divergent nucleases, for example, conserved amino acid sequences from Cluster 1 fused with conserved amino acid sequences from Cluster 4. In certain embodiments, methods other than identifying and mutating conserved sequences are used to alter nuclease function, for example, generating 3D structures to identify functional domains, using machine learning to identify functional domains, and/or conducting large- or small-scale mutagenesis screens followed by functional analysis of variants in vivo or in vitro.

In some embodiments, the nucleic acid-guided nuclease is expressed from bacterial or mammalian expression constructs and evaluated as recombinant or purified proteins. In some embodiments, functionality is determined by testing the ability to generate DNA double strand breaks and the induction of indel (insertion and deletion) mutations and loss of function (LOF) mutations in cells. In some embodiments, RNP complexes are generated by incubating guide nucleic acids with each nucleic acid-guided nuclease. In one particular embodiment, RNP complexes are generated by incubating 375 pmol of guide nucleic acids with 50 pmol of each nucleic acid-guided nuclease for 10 minutes. In some embodiments, RNP complexes are introduced into cells using electroporation or nucleofection and the cutting efficiency is measured by quantifying the frequency of insertion/deletion mutations in the edited population by performing Sanger sequencing and ICE (Inference of CRISPR Edits, online tool from Synthego) analysis on PCR amplicons containing the cut sites in genes of interest. In some embodiments, successful generation of LOF mutations is confirmed by measuring protein expression levels of targeted genes using western blot, flow cytometry, or ELISA.

### 6.3.3. Signal Peptide Fusions

In some embodiments, the nuclease polypeptide is fused to a fusion peptide. In the embodiments, the nucleic acid-guided nuclease comprises (1) a nuclease polypeptide and (2) a fusion peptide. The fusion peptide can be a signal peptide. The signal peptide can be a prokaryotic or eukaryotic signal peptide fused in-frame to the nuclease polypeptide. The fusion peptide can be fused to the N-terminus or C-terminus of the nuclease polypeptide. The fusion peptide can be fused in the middle of the nuclease polypeptide. In some embodiments, the fusion peptide is a reporter protein or a tag for purification of the endonuclease polypeptide. In some embodiments, the fusion peptide provides additional functional attributes including transcriptional activation, transcriptional repression, DNA or RNA base editing, recombinase/integrase activity, and nickase activity.

In some embodiments, the fusion peptide is a signal peptide.

In some embodiments, the signal peptide is fused in-frame to the C-terminus of the nuclease polypeptide. In some embodiments, the signal peptide is fused in-frame to the N-terminus of the nuclease polypeptide.

### NLS Fusions

In some embodiments, the nuclease polypeptide is fused to a one or more nuclear localization sequences (NLSs), such as about 1, 2, 3, 4, 5, 6, 7, 8, 9, 10, or more NLSs. In some embodiments, the nucleic acid-guided nuclease comprises 1, 2, 3, 4, 5, 6, 7, 8, 9, 10, or more NLSs at or near the amino-terminus, 1, 2, 3, 4, 5, 6, 7, 8, 9, 10, or more NLSs at or near the carboxy-terminus, or a combination of these (e.g., one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered to be near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

In some embodiments, an NLS called a monopartite NLS (PKKKRKV from the SV40 Large T-antigen, PAAKRVKLD from c-Myc, or KLKIKRPVK from TUS-protein) is fused at the N or C-terminus of the nuclease polypeptide. In some embodiments, an NLS called a bipartite or nucleoplasmin NLS (KR [PAATKKAGQA]KKKK) is fused at the N or C-terminus.

In some embodiments, the nuclease enzyme fused with the NLS is used for applications that require trafficking of the fusion enzyme to the nucleus of a cell, e.g., a mammalian cell. The fusion enzyme is therein used for engineering the genome of the cell.

### Functional Fusions

In some embodiments, the nuclease polypeptide is fused to a transcriptional activation domain. The transcriptional activation domain can be fused to the N- or C-terminus of the nuclease polypeptide. The fusion can be direct or via a linker. The fused transcriptional activation domain recruits the transcriptional preinitiation complex to the promoter of a gene resulting in RNA polymerase mediated expression. In some embodiments, the transcriptional activation domain is or is a variant of the VP16 protein of herpes simplex virus, the nuclear factor kappaB, 65 kDa subunit (p65), Rta (Epstein-Barr virus R transactivator), In some embodiments, multiple domains of the same type or combinations are included.

In some embodiments, the nuclease polypeptide is fused to a UDG inhibitor (UGI) domain. The UGI domain can be fused to the N- or C-terminus of the nuclease polypeptide. In some embodiments, the deaminase domain is fused to the C-terminus of the nuclease polypeptide. The fusion can be direct or via a linker. In some embodiments, the nuclease polypeptide is fused to a deaminase domain at the N-terminus, and a UGI domain at the C-terminus of the nuclease polypeptide. Uracil DNA glycosylases (UDGs) recognize uracil, inadvertently present in DNA and initiates the uracil excision repair pathway by cleaving the N-glycosidic bond between the uracil and the deoxyribose sugar, releasing uracil and leaving behind a basic site (AP-site). In some embodiments, the UGI domain is or is a variant of UGI from B. subtilis bacteriophage PBS1 or PBS2 (UniProtKB-P14739). In some embodiments, the nuclease polypeptide is fused to a factor involved in double strand break repair choice (e.g., CtlP, Mre11, and a truncated piece of p53 named DNIs).

### 6.4. Guide Nucleic Acid

In preferred embodiments, a guide nucleic acid complexes with a compatible nucleic acid-guided nuclease. In some embodiments, a nucleic acid-guided nuclease is used together with a heterologous guide nucleic acid.

In some embodiments, a nucleic acid-guided nuclease and a heterologous guide nucleic acid originate from two different species. In some embodiments, a nucleic acid-guided nuclease and a heterologous guide nucleic acid originate from the same species. In some embodiments, a nucleic acid-guided nuclease and a heterologous guide nucleic acid originate from the same species but does not present in the same cell in nature.

Compatibility of nucleic acid-guided nucleases and guide nucleic acids can be determined by empirical testing. Heterologous guide nucleic acids can come from different bacterial species or be non-naturally occurring, being synthetic or engineered.

In some embodiments, the guide nucleic acid is DNA. In some embodiments, the guide nucleic acid is RNA. In some embodiments, the guide nucleic acid comprises both DNA and RNA. In some embodiments, the guide nucleic acid comprises non-naturally occurring nucleotides. In cases where the guide nucleic acid comprises RNA, the RNA guide nucleic acid can be encoded by a DNA sequence.

In some embodiments, a guide nucleic acid comprises one or more polynucleotides. In some embodiments, a guide nucleic acid comprises a guide sequence capable of hybridizing to a target sequence, and a scaffold sequence capable of interacting with or complexing with a nucleic acid-guided nuclease. In some embodiments, a guide sequence and a scaffold sequence are in a single polynucleotide. In some embodiments, a guide sequence and a scaffold sequence are in two or more separate polynucleotides.

A guide nucleic acid can comprise a scaffold sequence. In general, a ‘scaffold sequence’ includes any sequence that has a sequence to promote formation of a ribonucleoprotein particle (RNP), wherein the RNP comprises a nucleic acid-guided nuclease and a guide nucleic acid. In some embodiments, a scaffold sequence promotes formation of the RNP by having complementarity along the length of two sequence regions within the scaffold sequence, such as one or two sequence regions involved in forming a secondary structure. In some cases, the one or two sequence regions are on the same polynucleotide. In some cases, the one or two sequence regions are on separate polynucleotides. Optimal alignment may be determined by any suitable alignment algorithm, and may further account for secondary structures, such as self-complementarity within either the one or two sequence regions. In some embodiments, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some embodiments, at least one of the two sequence regions is about or more than about 5, 6, 7, 8, 9, 10, 11, 12, 13, 14, 15, 16, 17, 18, 19, 20, 25, 30, 40, 50, or more nucleotides in length.

In some embodiments, a scaffold sequence of a guide nucleic acid comprises a secondary structure. A secondary structure can comprise a pseudoknot region. In some cases, binding kinetics of a guide nucleic acid to a nucleic acid-guided nuclease is determined in part by secondary structures within the scaffold sequence. In some cases, binding kinetics of a guide nucleic acid to a nucleic acid-guided nuclease is determined in part by nucleic acid sequence with the scaffold sequence.

In some embodiments, a guide nucleic acid comprises a guide sequence (i.e., a spacer sequence). A guide sequence is a polynucleotide sequence having sufficient complementarity with a target polynucleotide sequence to hybridize with the target sequence and direct sequence-specific binding of a complexed nucleic acid-guided nuclease to the target sequence. The degree of complementarity between a guide sequence and its corresponding target sequence, when optimally aligned using a suitable alignment algorithm, can be about or more than about 50%, 60%, 75%, 80%, 85%, 90%, 95%, 97.5%, 99%, or more. Optimal alignment can be determined with the use of any suitable algorithm for aligning sequences. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20 nucleotides in length. In preferred embodiments, the guide sequence is 10-30 nucleotides long. The guide sequence can be 15-20 nucleotides in length. The guide sequence can be 15 nucleotides in length. The guide sequence can be 16 nucleotides in length. The guide sequence can be 17 nucleotides in length. The guide sequence can be 18 nucleotides in length. The guide sequence can be 19 nucleotides in length. The guide sequence can be 20 nucleotides in length.

A guide nucleic acid can be engineered to target a desired target sequence by altering the guide sequence such that the guide sequence is complementary to the target sequence, thereby allowing hybridization between the guide sequence and the target sequence. A guide nucleic acid with an engineered guide sequence can be referred to as an engineered guide nucleic acid. Engineered guide nucleic acids are often non-naturally occurring.

In some embodiments, genome editing by the nuclease does not require or is not dependent on a trans-activating CRISPR RNA (tracr) sequence and/or direct repeat is 5′ (upstream) of the guide (target or spacer) sequence.

A targetable nuclease system that includes one or more non-naturally occurring guide nucleic acid is a non-naturally occurring system.

In some embodiments, a chemical modification that alter RNA stability, subcellular targeting, tracking (e.g., by a fluorescent label) is used to modify guide RNA. These modifications are useful for improving the function of novel endonucleases for specific applications.

In some embodiments, the guide nucleic acid comprises: phosphorothioate, inverted polarity linkages, and abasic nucleoside linkages, locked nucleic acid (LNA), peptide nucleic acid (PNA), morpholino nucleic acid, cyclohexenyl nucleic acid (CeNA); and modified sugar moieties selected from 2′-O-methoxyethyl, 2′-O-methyl, and 2′-fluoro,), 2′-dimethylaminooxyethoxy, 2′-dimethylaminoethoxyethoxy. Additional modifications include conjugation of polyamine, polyamide, polyethylene glycol, polyether, cholesterol moiety, cholic acid, thioether, thiocholesterol, 5′ cap (e.g., a 7-methylguanylate cap (m7G)) or 3′ polyadenylated tail (i.e., a 3′ poly(A) tail). Additional modifications include a 5-methylcytosine; a 5-hydroxymethyl cytosine; a xanthine; a hypoxanthine; a 2-aminoadenine; a 6-methyl derivative of adenine; a 6-methyl derivative of guanine; a 2-propyl derivative of adenine; a 2-propyl derivative of guanine; a 2-thiouracil; a 2-thiothymine; a 2-thiocytosine; a 5-propynyl uracil; a 5-propynyl cytosine; a 6-azo uracil; a 6-azo cytosine; a 6-azo thymine; a pseudouracil; a 4-thiouracil; an 8-haloadenin; an 8-aminoadenin; an 8-thioladenin; an 8-thioalkyladenin; an 8-hydroxyladenin; an 8-haloguanin; an 8-aminoguanin; an 8-thiolguanin; an 8-thioalkylguanin; an 8-hydroxylguanin; a 5-halouracil; a 5-bromouracil; a 5-trifluoromethyluracil; a 5-halocytosine; a 5-bromocytosine; a 5-trifluoromethylcytosine; a 5-substituted uracil; a 5-substituted cytosine; a 7-methylguanine; a 7-methyladenine; a 2-F-adenine; a 2-amino-adenine; an 8-azaguanine; an 8-azaadenine; a 7-deazaguanine; a 7-deazaadenine; a 3-deazaguanine; a 3-deazaadenine; a tricyclic pyrimidine; a phenoxazine cytidine; a phenothiazine cytidine; a substituted phenoxazine cytidine; a carbazole cytidine; a pyridoindole cytidine; a 7-deazaguanosine; a 2-aminopyridine; a 2-pyridone; a 5-substituted pyrimidine; a 6-azapyrimidine; an N-2, N-6 or 0-6 substituted purine; a 2-aminopropyladenine; a 5-propynyluracil; or a 5-propynylcytosine.

### 6.7. RNP

A ribonucleoprotein particle or RNP is a complex formed between nuclease-guided nuclease and guide nucleic acid described in the above sections. The nuclease-guided nuclease and the guide nucleic acid that are compatible can form an RNP having targetable nuclease activity. In preferred embodiments, the RNP can be used for gene editing.

In some embodiments, the nuclease-guided nuclease and the guide nucleic acid are a natural pair. In some embodiments, it is a complex of a nucleic acid-guided nuclease and a heterologous guide nucleic acid. In some embodiments, the heterologous guide nucleic acid is non-naturally occurring, being synthetic or engineered.

### 6.8. Polynucleotide

In another aspect, the present invention provides a polynucleotide encoding a nucleic acid-guided nuclease. In some embodiments, the polynucleotide encodes a naturally occurring nucleic acid-guided nuclease. In some embodiments, the polynucleotide encodes a non-naturally occurring nucleic acid-guided nuclease described herein. In certain embodiments, the non-naturally occurring nuclease is an engineered nucleic acid-guided nuclease described herein.

In some embodiments, the polynucleotide comprises a first polynucleotide segment encoding a nuclease polypeptide and a second polynucleotide segment encoding a fusion peptide. The fusion peptide can be a signal peptide or one or more NLSs.

In some embodiments, the polynucleotide comprises a first polynucleotide segment encoding the nucleic acid-guided nuclease having at least 95% sequence identity to a sequence selected from SEQ ID NO: 2-273.

In some embodiments, the polynucleotide has been codon optimized for expression in mammalian cells. In some embodiments, the first polynucleotide is codon optimized. In some embodiments, the polynucleotide has been codon optimized for expression in bacteria or eukaryote or yeast. In some embodiments, the polynucleotide has been codon optimized for expression in human cells.

In some embodiments, the first polynucleotide segment has a sequence having at least 95%, 96%, 97%, 98%, or 99% sequence identity to a sequence selected from SEQ ID NO: 722-766. In some embodiments, the first polynucleotide segment has a sequence selected from SEQ ID NO: 722-766.

In some embodiments, the first polynucleotide segment has a sequence having at least 95%, 96%, 97%, 98%, or 99% sequence identity to a sequence selected from SEQ ID NO: 677-721. In some embodiments, the first polynucleotide segment has a sequence selected from SEQ ID NO: 677-721.

In another aspect, the present disclosure provides a vector encoding the nucleic acid-guided nuclease provided herein. In some embodiments, the vector comprises the polynucleotide described herein. In some embodiments, the vector comprises at least one mRNA. In some embodiments, the vector further comprises a promoter or other regulatory element operably linked to the polynucleotide encoding the nucleic acid-guided nuclease. In some embodiments, the regulatory element drives expression in a tissue-specific (e.g., liver, brain, lymphocyte, muscle, tumor, virus-infected cells, etc.) or temporally specific manner (e.g., embryonic, fetal, cell cycle specific, etc.). In some embodiments, the vector is a plasmid. In some embodiments, the vector is a viral vector. In certain embodiments, the vector is an AAV, retrovirus, adenovirus, helper dependent adenovirus, or lentivirus (including IDLV). In certain embodiments, the means of delivery is a particle, nanoparticle, or lipid nanoparticle. In certain embodiments, the means of delivery is by exosomes or fusosomes. In certain embodiments, the means of delivery is a microbubble. In certain embodiments, the means of delivery is by electroporation. In some embodiments, expression constructs are introduced into target cells using electroporation or transfected using lipid or chemical-based methods, “gene-guns” using particle bombardment, microinjection, ligand mediated gene delivery, impalefection, laser irradiation, photoporation, sonoporation, hydroporation, and magnetofection.

In some embodiments, prokaryotic and eukaryotic expression constructs are designed to express both the nucleic acid-guided nuclease and guide nucleic acid in target cells. In some embodiments, expression constructs are for transient or stable expression in target cells. In some embodiments, constructs are designed to express a single or numerous guide nucleic acids in tandem.

In some embodiments, the nucleic acid-guided nuclease and guide nucleic acid are delivered as RNA.

In some embodiments, biological tools, or systems such as viral vectors are used to deliver the nucleic acid-guided nuclease and guide nucleic acid into target cells. In some embodiments, this involves the generation of vectors that produce viral particles in a helper cell line. Viral particles are collected and used to transduce the target cell line. In some embodiments, viral vectors are either integrating or non-integrating vectors such as lentiviral, adenoviral, and adeno-associated viral vectors. In some embodiments, these biological tools are used to introduce either or both the nucleic acid-guided nuclease and guide nucleic acid into target cells. In some embodiments, expression of both or either the nucleic acid-guided nuclease and guide nucleic acid is controlled using inducible expression vectors. In some embodiments, expression from vectors is controlled using cell type specific promoters to drive either or both the nucleic acid-guided nuclease and guide nucleic acid expression in specific cell types. This allows for systemic delivery of viral particles but restricts expression to specific cell type in an organism.

### 6.9. Host Cell

In yet another aspect, the present disclosure provides a host cell comprising the nucleic acid-guided nuclease provided herein. In some embodiments, the host cell comprises a polynucleotide encoding a nucleic acid-guided nuclease. In some embodiments, the host cell comprises a vector comprising a polynucleotide encoding a nucleic acid-guided nuclease.

In some embodiments, the nucleic acid-guided nuclease is a naturally occurring protein. In some embodiments, the nucleic acid-guided nuclease is a synthetic or engineered protein.

In some embodiments, the host cell further comprises a guide nucleic acid. In some embodiments, the guide nucleic acid is a heterologous guide nucleic acid. In some embodiments, the host cell comprises an expression construct encoding a guide nucleic acid. In some embodiments, a guide nucleic acid is provided in a cassette in a single polynucleotide with the polynucleotide encoding a nucleic acid-guided nuclease.

The host cell can be transiently or non-transiently transfected with one or more vectors, linear polynucleotides, polypeptides, nucleic acid-protein complexes, or any combination thereof.

In some embodiments, the host cell is a prokaryotic cell. In some embodiments, the host cell is a eukaryotic cell. Reference is made to PCT/US13/74667, incorporated herein by reference. In some embodiments, modifications are made to germline cells, resulting genetically engineered multicellular organisms, for example a knock-in or knock-out mouse, rat, or human. See Platt et al., Cell, 159 (2): 440-455, which is incorporated herein by reference. In some embodiments, the host cell may be a non-mammalian eukaryotic cell such as a poultry bird (e.g., chicken), a vertebrate fish (e.g., salmon), shellfish (e.g., oyster, clam, shrimp), insect (e.g., fruit fly), yeast, or plant (e.g., cassava, corn, sorghum, soybean, oat, rice, citrus, nut trees, cotton, tobacco, edible fruits, edible vegetables, coffee, cocoa), such that a cell, tissue, or full organism is edited using the nuclease.

### 6.10. Targetable Nuclease System

In one aspect, the present disclosure provides a targetable nuclease system for editing a target region of a eukaryotic or prokaryotic genome, comprising (1) a nucleic acid-guided nuclease, and (2) a guide nucleic acid for complexing with the nucleic acid-guided nuclease. In some embodiments, the system further comprises (3) a homology template configured to bind to the target region.

### 6.10.1. Nucleic Acid-Guided Nuclease

The gene editing system comprises a nucleic acid-guided nuclease described herein. The nucleic acid-guided nuclease can be a naturally occurring nuclease or an engineered nuclease.

The targetable nuclease system can comprise any of the nucleic acid-guided nuclease described herein. In some embodiments, the nucleic acid-guided nuclease comprises a nuclease having at least 95% sequence identity to a sequence selected from SEQ ID NO: 2-273.

In some embodiments, the nucleic acid-guided nuclease is originated from Acidaminococcus massiliensis, Acidaminococcus sp., Acinetobacter indicus, Agathobacter rectalis, Anaerovibrio lipolyticus, Bacteroidales bacterium, Bacteroides galacturonicus, Bacteroides plebeius, Bacteroidetes bacterium, Butyrivibrio fibrisolvens, Butyrivibrio hungatei, Butyrivibrio sp., Candidatus Falkowbacteria bacterium, Candidatus Falkowbacteria bacterium, Candidatus Gottesmanbacteria bacterium, Candidatus Jacksonbacteria bacterium, Candidatus Magasanikbacteria bacterium, Candidatus Moranbacteria bacterium, Candidatus Pacebacteria bacterium, Candidatus Roizmanbacteria bacterium, Candidatus Ryanbacteria bacterium, Candidatus Saccharibacteria bacterium, Candidatus Sungbacteria bacterium, Candidatus Uhrbacteria bacterium, Candidatus Wildermuthbacteria bacterium, Candidatus Yonathbacteria bacterium, Catenovulum sp., Clostridiales bacterium, Clostridium sp., Coprococcus eutactus, Coprococcus sp., Deltaproteobacteria bacterium, Elizabethkingia sp., Eubacteriaceae bacterium, Eubacterium eligens, Eubacterium rectale, Eubacterium sp., Eubacterium ventriosum, Fibrobacter sp., Fibrobacter succinogenes, Firmicutes bacterium, Flavobacterium branchiophilum, Francisella hispaniensis, Francisella novicida, Francisella philomiragia, Francisella tularensis, Lachnospiraceae bacterium, Lachnospira pectinoschiza, Lentisphaeria bacterium, Leptospiraceae bacterium, Leptospira sp., Moraxella bovis, Moraxella bovoculi, Moraxella lacunata, Moraxella ovis, Moraxella sp., Muribaculaceae bacterium, Patescibacteria group bacterium, Phycisphaerae bacterium, Phycisphaerales bacterium, Porphyromonadaceae bacterium, Porphyromonas crevioricanis, Prevotella brevis, Prevotellaceae bacterium, Prevotella copri, Prevotellamassilia sp., Prevotella ruminicola, Prevotella sp., Prolixibacteraceae bacterium, Pseudobutyrivibrio sp., Pseudobutyrivibrio xylanivorans, Psychrobacter sp., Ruminococcaceae bacterium, Ruminococcus sp., Sedimentisphaera cyanobacteriorum, Sneathia amnii, Spirochaetia bacterium, Succinivibrionaceae bacterium, or Treponema sp. In some embodiments, the nucleic acid-guided nuclease is a variant or a modification of a naturally occurring nucleic acid-guided nuclease. In some embodiments, the nucleic-acid guided nuclease comprises a nuclease polypeptide and a fusion peptide. The fusion peptide can be a signal peptide or one or more NLSs.

In some embodiments, the nucleic acid-guided nuclease is produced from a codon-optimized polynucleotide.

### 6.10.2. Guide Nucleic Acid

The gene editing system further comprises a guide nucleic acid described herein. The guide nucleic acid can be naturally occurring, synthetic, or engineered.

In some embodiments, the engineered guide polynucleotide is designed to form a complex with the nuclease and comprises a guide sequence, wherein the guide sequence is designed to hybridize with a target sequence in a prokaryotic or eukaryotic cell.

In some embodiments, a nucleic acid-guided nuclease is used together with a heterologous guide nucleic acid, which is compatible with the nucleic acid-guided nuclease, thereby forming a functional RNP.

In some embodiments, a nucleic acid-guided nuclease and a heterologous guide nucleic acid originate from two different species. In some embodiments, a nucleic acid-guided nuclease and a heterologous guide nucleic acid originate from the same species. In some embodiments, a nucleic acid-guided nuclease and a heterologous guide nucleic acid originate from the same species but does not present in the same cell in nature.

### 6.10.3. Homology Template

A homology template includes a sequence homologous to a target sequence. The target sequence can be any polynucleotide endogenous or exogenous to a prokaryotic or eukaryotic cell. For example, the target sequence can be a polynucleotide residing in the nucleus of the eukaryotic cell. In some embodiments, the target region is in a eukaryotic cell genome. In some embodiments, the target region is in a bacterial cell genome. In some embodiments, the target region is in a plant cell genome. In some embodiments, the target region is in a mammalian cell genome. In some embodiments, the target region is in a human genome.

The target sequence can be a coding sequence or a non-coding sequence. The target sequence can be localized close to or include a PAM; that is, a short sequence recognized by an RNP. In some embodiments, PAMs are 2-5 base pair sequences adjacent the target sequence. A PAM can be 1, 2, 3, 4, 5, 6, 7, 8, 9, 10 or more nucleotides in length.

In some embodiments, the PAM sequence is TTTV (wherein V is any one base selected from A, C, or G). In some embodiments, the PAM sequence is not TTTV. In some embodiments, the PAM sequence is selected from NTTN, NCTV, CTTV, GTTV, NCTV, TCTV, and NTTV (wherein N is any one base selected from A, T, C, or G).

A homology template can comprise at least one mutation or a modification relative to a target sequence. In some embodiments, the homology template comprises an insertion, deletion, or modification compared to the target region. The homology template can comprise a sequence complementary to the target region.

A homology template can comprise a homology region (or homology arms) flanking at least one mutation or a modification relative to a target sequence, such that the flanking homology regions facilitate homologous recombination of the editing sequence into a target sequence. In some embodiments, the at least one mutation is one or more PAM mutations that mutate or delete a PAM site. A PAM mutation can be a silent mutation. A PAM mutation can be a non-silent mutation. Non-silent mutations can include a missense mutation. An editing sequence can comprise one or more mutations in a coding or non-coding sequence relative to a target site.

In some embodiments, the homology template comprises at least one mutation relative to a target sequence. A mutation can be a silent mutation or non-silent mutation, such as a missense mutation. A mutation can include an insertion of one or more nucleotides or base pairs. A mutation can include a deletion of one or more nucleotides or base pairs. A mutation can include a substitution of one or more nucleotides or base pairs for a different one or more nucleotides or base pairs. Inserted or substituted sequences can include exogenous or heterologous sequences. In some embodiments, the homology template further comprises an exogenous sequence flanked by homology regions.

In some embodiments, homology regions within the homology template flank the one or more mutations of the editing cassette and can be inserted into the target sequence by recombination. Recombination can comprise DNA cleavage, such as by a nucleic acid-guided nuclease, and repair via homologous recombination.

In some embodiments, a homology template is in a vector or provided as a separate polynucleotide, such as an oligonucleotide, linear polynucleotide, or synthetic polynucleotide. In some embodiments, a homology template is on the same polynucleotide as a guide nucleic acid. In some embodiments, a homology template is on a separate polynucleotide as a guide nucleic acid. In some embodiments, a homology template is designed to serve as a template in homologous recombination, within or near a target sequence nicked or cleaved by a nucleic acid-guided nuclease. A homology template can be of any suitable length, such as about or more than 10, 15, 20, 25, 50, 75, 100, 150, 200, 500, 1000, or more nucleotides in length. In some embodiments, a homology template is complementary to a portion of a polynucleotide comprising the target sequence. A homology template can overlap with one or more nucleotides of a target sequences (e.g., about or more than about 1, 5, 10, 15, 20, 25, 30, 35, 40, 50, 100, 150, 200, 250, 300, 400, 500, 600, 700, 800, 900, 1000, or more nucleotides).

### 6.11. Method of Editing a Genome

In one aspect, the present disclosure provides a method of modifying a target region of a eukaryotic or prokaryotic genome using a gene editing system provided herein. The method can comprise the steps of (1) contacting a sample comprising the target region with (i) a nucleic acid-guided nuclease and (ii) a guide nucleic acid complexed with the nucleic acid-guided nuclease and (2) allowing the nucleic acid-guided nuclease to modify the target region. In some embodiments, the sample is further contacted with (iii) a homology template configured to bind to the target region.

In some embodiments, the sample comprises a eukaryotic cell, a bacterial cell, a plant cell, a mammalian cell or a human cell. In some embodiments, the sample comprises an immune cell. In some embodiments, the immune cell is a B cell or T cell. In some embodiments, the cell for genome editing is a germline cell, which results in a transgenic multicellular organism, such as a human, mouse, or rat. In some embodiments, the cell for genome editing is a stem cell, hematopoietic stem cell, induced pluripotent stem cell, or other such target cell which allows for nuclease-mediated genome editing followed by derivation of specific cell or tissue types.

In some embodiments, one or more vectors encoding one or more components of a gene editing system are introduced into a host cell. In some embodiments, a nucleic acid-guided nuclease and a guide nucleic acid are operably linked to separate regulatory elements on separate vectors. In some embodiments, two or more of the elements expressed from the same or different regulatory elements combined in a single vector are introduced. When several elements are combined in a single vector, the coding sequence of one element may be located on the same or opposite strand of the coding sequence of a second element, and oriented in the same or opposite direction. In some embodiments, a single promoter drives expression of a transcript encoding a nucleic acid-guided nuclease and one or more guide nucleic acids. In some embodiments, a nucleic acid-guided nuclease and one or more guide nucleic acids are operably linked to and expressed from the same promoter. In other embodiments, one or more guide nucleic acids or polynucleotides encoding the one or more guide nucleic acids are introduced into a cell in the in vitro environment already comprising a nucleic acid-guided nuclease or polynucleotide sequence encoding the nucleic acid-guided nuclease. Delivery vehicles, vectors, particles, nanoparticles, formulations, and components thereof for expression of one or more elements of the nucleic acid targeting system are as used elsewhere, e.g., PCT/US2013/074667 incorporated by reference herein.

In some embodiments, the method comprises the step of contacting more than one guide nucleic acid. In some embodiments, each of the more than one guide nucleic acid has a different guide sequence, thereby targeting a different target sequence.

In some embodiments, the method is used for modifying a target region in a prokaryotic or eukaryotic cell in vivo, ex vivo, or in vitro. In some embodiments, the method comprises sampling a cell or a population of cells such as prokaryotic cells, or those from a human or non-human animal or plant for gene editing. Culturing may occur at any stage in vitro or ex vivo. The cell or cells may even be re-introduced into the host.

In some embodiments, the method comprises the step of allowing a RNP to bind to the target sequence to effect cleavage of said target region, thereby modifying the target region.

The present invention relates to engineering and optimization of systems, methods, and compositions used for the control of gene expression involving DNA or RNA sequence targeting, that relate to the nucleic acid targeting system and components thereof. In some embodiments, viral vectors are used to deliver libraries of nuclease-guided nucleases and or libraires of guide nucleic acids into target cells. In some embodiments, bacteria such as Agrobacterium tumefaciens are used to transfer the sequences for nuclease-guided nuclease and the guide nucleic acid into the plant genome.

In some embodiments, these methods are used to introduce the nuclease-guided nuclease and guide nucleic acid sequences into prokaryotes and eukaryotes including but are not limited to bacteria, yeast, fungi, nematodes, drosophila, zebrafish, mice, rats, primates, and other animal model systems.

In some embodiments, the sequences for the nucleic acid-guided nuclease and guide nucleic acid are delivered into target cells using the systems described above to make knock out (KO) or LOF mutations by inducing DNA double stranded breaks (DSBs).

In some embodiments, the sequences for the nucleic acid-guided nuclease and guide nucleic acid are delivered with homology directed repair (HDR) templates for making knock-in (KI) mutations. In some embodiments, HDR templates are provided as either single stranded or double stranded DNA and are introduced simultaneously with the nucleic acid-guided nuclease and guide nucleic acid, or sequentially using the methods for gene transfer described above. In some embodiments, HDR templates are engineered to incorporate naturally occurring or synthetic sequences into the target genome.

In some embodiments, modified versions of the nucleic acid-guided nuclease are generated to make a “CRISPR-Nickase” which results in a DNA single strand break. In some embodiments, this alternation may enhance fidelity and decrease off target editing and is used for generating KO or KI mutations.

### 6.12. Genome-Edited Cells

In one aspect, the present disclosure provides a cell comprising a genome modified by the method described herein. In some embodiments, the cell is an immune cell. In some embodiments, the cell is B cell or T cell.

An advantage of the present invention is that it minimizes or avoids off-target binding and its resulting side effects while editing the genome of cells.

In some embodiments, a disease associated gene or polynucleotide has been modified. In some embodiments, a polynucleotide encoding a B cell or T cell receptor has been modified.

In some embodiments, the gene editing comprises knocking out genes, modifying gene regulatory sequences to increase or decrease RNA expression, editing genes, altering genes, amplifying genes, replacing genes, inserting genes, and repairing particular mutations.

In some embodiments, the engineered nucleic acid-guided nuclease is an enzymatically dead nuclease which is used to block transcription of a target gene without altering the host cells genomic DNA sequence.

In some embodiments, an enzymatically dead nucleic acid-guided nuclease is fused to transcriptional activators to enhance transcription of a target gene without altering the host cells genomic DNA sequence.

In some embodiments, a library of sequences for the nucleic acid-guided nuclease or guide nucleic acid is introduced into cells to alter gene expression to identify gene functions at the genome level. In some embodiments, these screens may result in novel biological insights or the identification of novel drug targets.

In some embodiments, an enzymatically dead nucleic acid-guided nuclease and a guide nucleic acid are utilized for Chromatin immuno-precipitation (ChIP) of regions of the genome. In some embodiments, the nuclease is fused to a protein tag to allow for binding and purification of specific regions of chromatin. In some embodiments, these tags include the hemagglutinin (HA) domain, an IF2 domain, a GST domain, a green florescent protein domain, and a 6×His tag. In some embodiments, these proteins and are used for epigenetic, genomic, and proteomic profiling of specific chromatin regions.

In some embodiments, an enzymatically dead nucleic acid-guided nuclease is fused to enzymes which modify or label DNA. In some embodiments, enzymes such as methyltransferases, demethylases, acetyltransferases, and deacetylases are used to add or remove modifications to the target cell genome.

In some embodiments, an enzymatically dead nucleic acid-guided nuclease is fused to florescent proteins to visualize chromatin dynamics during cellular processes such as DNA replication. In some embodiments, the enzymatically inactive nucleic acid-guided nuclease florescent reporter is used to detect specific nucleic acids within live or dead cells.

In some embodiments, the nucleic acid-guided nuclease used as a diagnostic to detect viral pathogens or microbial contaminants in biological samples. In some embodiments, the nucleic acid-guided nuclease is enzymatically active or enzymatically dead and is used to detect nucleic acids using enzymatic reporters such as horseradish peroxidase, alkaline phosphatase, or florescent reporters such as green fluorescent protein.

In some embodiments, the nucleic acid-guided nuclease and guide nucleic acid are introduced into germ cells, gametes, zygotes, blastomeres, and embryonic stems to generate genetically engineered multi-cellar organisms for basic research or disease modeling. In some embodiments, the nucleic acid-guided nuclease and guide nucleic acid are introduced into cells using in vitro, ex vivo, or in vivo methods. In some embodiments, modified organisms are fungi, plants, and eukaryotes.

In some embodiments, the nucleic acid-guided nuclease and guide nucleic acid are introduced into somatic cells in vivo to generate genetically engineered multi-cellar organisms. In some embodiments, modified organisms are fungi, plants, and eukaryotes. In some embodiments, these approaches would aim to modify specific cell types or all cells within a developed or developing organism.

In some embodiments, the nucleic acid-guided nuclease and guide nucleic acid are introduced by intravenous injections, retro-orbital injection, intratracheal injection, intratumoral injection, joint and soft tissue injections, intra-muscular injection, intralesional injection, intraocular injection, or other methodologies for delivering nucleic acids, viral vectors, or RNA & protein complexes into tissues within a living organism.

In some embodiments, these methods are used for cancer or disease modeling, cell biological or genetic research, correction of disease associated mutations, cell therapies, wound healing and regeneration, diagnostics, imaging tools, agricultural purposes, drug discovery, and drug development and manufacturing.

In some embodiments, primary cells from patients are obtained and the nucleic acid-guided nuclease and guide nucleic acids are delivered into cells ex vivo. In some embodiments, cells are modified to contain any of the genomic, epigenomic, or transcriptomic alterations described above. In some embodiments, modified cells are introduced back into patients for therapeutic purposes. In some embodiments, these modifications either correct disease associated mutations or introduce sequences to enhance the regeneration or health promoting capacity of immune cells. In some embodiments, the engineered cells are used as therapeutics for cancer, autoimmunity, or infectious disease. In some embodiments, the target cells are T cells, natural killer cells, antigen presenting cells, macrophages, and hematopoietic stem cells. In some embodiments, T cell receptors (TCRs) or chimeric antigen receptors (CARs) are introduced into immune cells (e.g., T cells) from the patient and infused back into the patient. In some embodiments, the Cas nucleases are used to KO genes, e.g., endogenous TCRs, human leukocyte antigens, or immune suppressive genes. In some embodiments, the target cells are allogeneic (i.e., from a donor rather than the patient). In some embodiments, molecular switches, kill switches, or secretory or membrane bound proteins which facilitate tumor infiltration are introduced into the engineered cells.

### 6.13. Examples

### 6.13.1. Example 1: Identification and Computational Analysis of Nucleic Acid-Guided Nucleases in Prokaryotic Genomes

MAD7 is a Cas12a variant with only 31% homology with the canonical AsCpf1 from Acidominococcus species at the amino acid level and has evolved further away from Cas9 compared to AsCpf1. MAD7 amino acid sequence was used as query to search for homologs within other prokaryotes. blastx was used to query against 134,655 prokaryotic genomes in the NCBI Genbank database. 381 MAD7 homologs, which we term the “GIG-” nucleases or “GIG-” enzymes or “GIG-” Cas nucleases or “GIG-” Cas enzymes, were identified in this computational search. In an effort to obtain full-length coding sequences, the homolog sequences were extended upstream to the nearest methionine (if present), and downstream to the nearest stop codon (if present). These homologs had an average of 44.2% (range 22.96%-98.89%) identity to MAD7 amino acid sequence (FIG. 1). The homolog protein sequences were aligned to MAD7 using Clustal Omega (see Sequence Listing) and a phylogenetic tree was generated (FIG. 2).

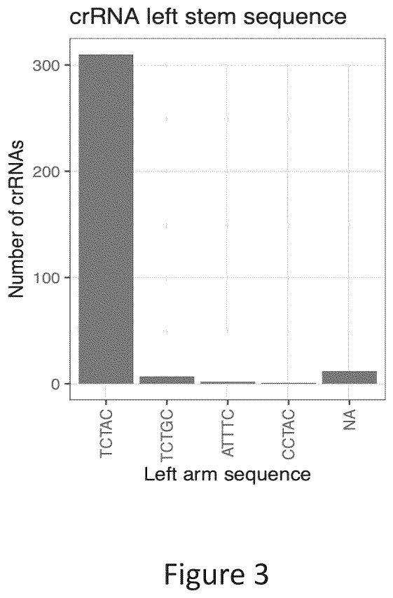

In the same genomes where the nuclease sequences were discovered, CRISPR repeat arrays were searched for using PILER-CR (Edgar BMC Bioinformatics 8:18, 2007). When transcribed, the CRISPR repeats form the CRISPR RNA (crRNA) containing a stem loop structure and the spacer region for sequence-specific targeting. Palindromic sequences were searched for within the CRISPR repeat sequences using the findPalindromes function of the R package, Biostrings, using stem loop arm length of 5 nucleotides, and loop length within 3 to 5 nucleotides. The majority of the predicted crRNAs contained the canonical stem loop left arm sequence of TCTAC, with a minority of them containing novel stem loop left arm sequences of TCTGC, ATTTC or CCTAC (FIG. 3). The CRISPR repeat sequences are listed as SEQ IDS 274-627.

To identify conserved domains in the MAD7 homologs, clusters were identified within the list of MAD7 homologs. Using the R packages ape and geiger, 12 clusters were identified containing varying number of homolog sequences (FIG. 2). A multiple sequence alignment was performed and consensus amino acid sequences were generated for sequences within each cluster, as provided in Table 1.

Furthermore, among the consensus sequences, conserved domains were identified as strings (i.e., peptides) containing ≥4 amino acids with ≤10% ambiguous amino acids and no gaps. These conserved peptide sequences, which may represent domains of functional importance to the GIG-Cas enzymes, are listed in Table 2. Non-conserved amino acids in the conserved domains are marked as Xs.

TABLE 1
Alignment of 272 MAD7 GIG- nuclease homologs with ClustalW.

| | GIG- | | |
|---|---|---|---|
| | nuclease | | |
| | SEQ ID | | |
| Cluster | NO | Position | Consensus sequence |
| 1 | 188, 204, | 1-49 | -----XXX-XNNFXXFIG---IXSXXKTLRNELIP-TXXTQEXIEKNX- |
| | 221, 256, | 50-98 | ---------IXXEDELRAENXQXXKXIXDDYXRXFIXEXLS-------- |
| | 240, 233, | 99-147 | --------------IXDIDWXXLFEAMEXXLKX--XD------------ |
| | 189, 202, | 148-196 | ----------XKXXLEKEQAEKRKXIYKKXXDDDRFKXXFXAKLISXXL |
| | 185, 247, | 197-245 | PEFXXXN--XX-----------XKEEKXEAXKLFXXFATXFXXXFKNRK |
| | 191, 201, | 246-294 | NXXSAXAISXSICXRIVNXNXPXFLSNXLVXXRIXKNXP-XXIXKIEXE |
| | 246, 81, | 295-343 | LKDXLX--------XXSLEEIXSXXFYXXVXTQXG------IDFYNDIC |
| | 83, 243, | 344-392 | GXXNXXMNLYCQXXKNXK-X-------X---------NXXKMRKLHKQI |
| | 88, 258, | 393-441 | LXXRETSSEXPXKFESDEEVYXSVNXFL--------DNXXSKNIXERLR |
| | 223, 131, | 442-490 | KXGXNXNXYX--LDKIYIXXKXYXXVSQKXYGXWXTINXALEXXYXNXX |
| | 214, 226, | 491-539 | XGKGKSKXXKV-----KKAVKXDXXKSXXEXNELVXXYXX--------- |
| | 85, 231, | 540-588 | -------------------------X-KAEXYIXEISXILXXXEXXELK |
| | 79, 80, | 589-637 | Y--NPXIXLIENEEKAXEXKNXLDXIMNXFHWCXVFXXE--EEVXKDNN |
| | 217, 238, | 638-686 | FYAELEEIYDELXP-------XXSLYNXVRNYVTQKPYSTK-KIKLNFG |
| | 87, 254, | 687-735 | IPTLADGWSKXKEYDNNAIILXXDX----XYYLGIFNAKX-XXDKKIIE |
| | 248, 241, | 736-784 | G------------------------SEXXGDYKKMXYNLLPGP------ |
| | 242, 65, | 785-833 | ----------------NKMXP--------XVFLSSKTGXETYKPS---- |
| | 94, 95, | 834-882 | -------------XYILEGYXQNK-------XXXX-------------- |
| | 143, 176, | 883-931 | --------SKNFDIKFCX-DLIDXXKX-----CIAKHPXWKNFGFKFS- |
| | 17, 169, | 932-980 | --DTSX---------------YEDISGFYREVEKQGYKIDXTYISEKDI |
| | 165, 160, | 981-1029 | XXLVEXGXLYLFQIYNKDFXEXSTG------KDNLHTXYXKNLFS---- |
| | 172, 157, | 1030-1078 | ----EENLKXIVLKLNGEAELFXR----KSSIK--KPXIHKKGSILVNX |
| | 166, 163, | 1079-1127 | TYKXXEX---------XXX-IPXXIYQEXYKYFN----KXXXELSDEAK |
| | 10, 16, | 1128-1176 | KXKD--KVGHKEAXHXIVKDXRYTXD-KXFXHXPITINFKA---XK-XX |
| | 122, 126, | 1177-1225 | INXRVLXYXAKNP-DXXXIGIDRGERXLIYXSXIXXXGX-IXEQKSFNI |
| | 139, 144, | 1226-1274 | VNG----------YXYQEKLKQREXERDXARKXWXEIGKIKXLKEGYLS |
| | 145, 23, | 1275-1323 | LVXHEIAKXXXXYNAIXXMEDLN-YGFKRGRF-KVERQVYQKFETMLIN |
| | 155, 123, | 1324-1372 | KLNYLVFKDRX-XXENGGXLXGYQLTYIPESLKNXGKQCGXXFYVPAAY |
| | 137, 138, | 1373-1421 | TSKIDPTTGFVNIFNFKDLTNXX------KXKEFLXKFDSIRYDXEKX- |
| | 18, 48, | 1422-1470 | ---------LFXFTFDYNNFKTXN------------------XXMXX-X |
| | 125, 127, | 1471-1519 | WTVYTYG-ERIXRXFXX------------XRXXXXSXXIDPTXXXXKXX |
| | 128, 135, | 1520-1568 | E----XXXINXXDGHDXRXDIXDXEXX---------XXX-FXXXXFEIF |
| | 136, 150, | 1569-1617 | XLTXQMRNSLXX----XX-D----XDXXISPVLN-XNXXFXDSXXXXXK |
| | 153, 1, 59, | 1618-1666 | ------------LPKDADANGAYXIALKGLYXXKQIKXXXKEDGKFXRX |
| | 15, 134, | 1667-1694 | XLKISNXDWFDFIQNKRYL--------- |
| | 171, 32, | | |
| | 175, 184, | | |
| | 159, 156, | | |
| | 199, 147, | | |
| | 146, 149, | | |
| | 154, 148, | | |
| | 198, 60, | | |
| | 120, 19, | | |
| | 197, 161, | | |
| | 173, 174, | | |
| | 50, 49, | | |
| | 196, 5, | | |
| | 130, 3, | | |
| | 200, 74, | | |
| | 97, 177, | | |
| | 33, 41, 86 | | |
| 2 | 228, 236, | 1-51 | XXXXEXXTXXXXXXKXXXXELXXXGKTXXXIXXXGXXXXDXXXXXXXXXXX |
| | 8 | 52-102 | KXXIDXXXXXXXXXXLXXXXXDXXXXXXXXXXXXXXXXXXXXXXXXXXLXX |
| | | 103-153 | XXXXAXPXXXXXXXXXXXXXXLQXXXXXXXXXXXXXXXXXXXXXXXXXXXX |
| | | 154-204 | XXXXXXXXXXFXXXXIXXXXXXTXXXXXXXXXXXXXXXXXNXXXXXXIXXX |
| | | 205-255 | XXXXXXNXXXXXXLXXXXXXXXXXXXXXXXDXXNXXXXXXIXXXXXXXXXL |
| | | 256-306 | XXXXXEYXXXXXXXXXXXXXXXXXXQLXKQXLXXXXXXXXXXXXXTXDEXX |
| | | 307-357 | XVXXXXXXXXXXXXXXXLXXXXXXXXXXXGXXXXXXXXXLXXXXXXXXGXX |
| | | 358-408 | KXXXDXXXXXXXXXXXEXLXXXXXXXXXXXXXXXXXXXXXXXXXXXXXXXX |
| | | 409-459 | XLXXXXXXXXXXXXXXXKXXXXXXXXXXXXXXXEAXXXXXXXXXXXXXXXX |
| | | 460-510 | XXXXXXXXXXXXXXXXXAXXXXXXXXXXXXXXXXXEXXXXXFYXXFXXIXX |
| | | 511-561 | XXXXXXXXXXXVRNXXTKXXXXXXXXXXXXXXXXAXXXXXXWXXXXXXXXX |
| | | 562-612 | XXXXXKXDXKYYXXXXXXXXXXXXXXXXXXXXXXXXXXXXXXXXXXXXXXX |
| | | 613-663 | XXXXXXXXXXXXXXXXXXXXXDXXXXXXXXXXXXXXXXXXXXXXXXXXXXX |
| | | 664-714 | XXXXXXXXXXXXXXXXIXXXXXXXXXXXXYXXXXXXXXXXXXXYXDXXXFX |
| | | 715-765 | XXXXXXXXXXXXXXXIXXXXXXXLVDXGXXXLFXXXXKXXXXXXXXXXXXX |
| | | 766-816 | XXXXXXXXXXXXXXXXXXXXXXXXXXXXXXXXXXXXXXXXXXXXXNXXXXX |
| | | 817-867 | XXXXXXXXXEXXXXXXNXXSXXXXXXXXXXXXYXRXXXXXXXXXXXXXXXX |
| | | 868-918 | XXYXXXKXXLXXXXXXNXXXXXXVXXXXNXXVXEXXQXGXXXXIXXXXRXX |
| | | 919-969 | XXXXYXXVLXXXXXIXXXXSLNVIXXVDYXXLLXXXXXXXXXXXXXWXXXX |
| | | 970-1020 | XXXXLKXXYXXXAXXXXXXLXRXYNAXXXXEXXXXXXXXXXXXIDXQXXXX |
| | | 1021-1071 | FEXXXXXXLXXXXXKXVXDXXXXGSXXXXLQLXXXXXXXXXXXXXQXGILF |
| | | 1072-1122 | FXXXXYTXXIDPXXGFXXLFDXXXXXXXXXXXKXFXXXXXXSYXXDXXXFX |
| | | 1123-1173 | FXXXXXXVXXXTXXXXXXXXXXWXXXXXGXXXXXXXXXXXXXXXXXXXXXX |
| | | 1174-1224 | XXXKXXXXXXXIXXXXXXDLXXXXXXXXXXXDXXXXXXXXFXXXXXRXXXX |
| | | 1225-1275 | XXTXXXXXXXXXXXXXXXXXXXXXXXXXXKXPXXGXXXXXYNIXXXXXXXX |
| | | 1276-1306 | XXLXXXXXXXLXXXXXXXXXXXEXXXXXXXX |
| 3 | 245, 272 | 1-51 | XXXXXXXXXXXXXXXXXXXXXXXXXXXXXXXXXXXXXXXXXXXXXXXXXXX |
| | | 52-102 | XXXXXXXXXXXXXXXXXXXXXXXXXXXXXXXXXXXXXXXXXXXXXXXXXXX |
| | | 103-153 | XXXXXXXXXXXXXXXXXXXXXXXXXXXXXXXXXXXXXXXXXXXXXXXXXXX |
| | | 154-204 | XXXXXXXXXXXXXXXXXXXXXXXXXXXXXXXXXXXXXXXXXXXXXXXXXXX |
| | | 205-255 | XXXXXXXXXXXXXXXXXXXXXXXXXXXXXXXXXXXXXXXXXXXXXXXXXXX |
| | | 256-306 | XXXXXXXXXXXXXXXXXXXXXXXXXXXXXXXXXXXXXXXXXXXXXXXXXXX |
| | | 307-357 | XXXXXXXXXXXXXXXXXXXXXXXXXXXXXXXXXXXXXXXXXXXXXXXXXXX |
| | | 358-408 | XXXXXXXXXXXXXXXXXXXXXXXXXXXXXXXXXXXXXXXXXXXXXXXXXXX |
| | | 409-459 | XXXXXXXXXXXXXXXXXXXXXXXXXXXXXXXXXXXXXXXXXXXXXXXXXXX |
| | | 460-510 | XXXXXXXXXXXXXXXXXXXXXXXXXXXXXXXXXXXXXXXXXXXXXXXXXXX |
| | | 511-561 | XXXXXXXXXXXXXXXXXXXXXXXMIYTSQSEEEKNKNYKISFKYINSAKID |
| | | 562-612 | EMVEKDEMYLFQIYNKDFSSYSNGNYKLNTMYWNNIFDSNNIENIIFNIAG |
| | | 613-663 | GATIFFRAKSLERRETHKQGEVLSNKNKDNQKTTSIFSYPLIKNKRYTEDK |
| | | 664-714 | IKLHVPIEINCNSKNLNQRKLNYRVNKNIQNLKEVNIIGISRGTNDLLYAT |
| | | 715-765 | VINSKGEIIEQTSLGKIKNVVFDKSINATREIISDYNKIIDTKEKERENAN |
| | | 766-816 | NRNKTGEELNLEKSIKDIKNGYISQAVNVIANLVKKYNAIIVFEDLNDKKS |
| | | 817-867 | EKIESKIEKSIYRNIQNAVITKLSYLVDKKKKDKFAEGSILNGYQLTYYCE |
| | | 868-918 | QDLVEKQEDSEEKQNGIVFYVSTYMTTNIDPKTGFVNCFSVSLPKSIDGVK |
| | | 919-969 | DFLEKFKEIKFNTKEGYYEFVVNYENISKYTRYFIGEKLSKKEWVICSYGD |
| | | 970-1020 | RIEESKDINTGKTKYKKIDLTKEFKCLFEKFNIGQGKKLKTNIIKFLENLD |
| | | 1021-1071 | LGSGLQAKIKGTNRFINKQSIQQEEFCREFMRLFALILKLQNSDGKESYII |
| | | 1072-1122 | SPVKDSDGKFFDSRKHNEYSSIVPENEISNSAYNIARKGLLIINRIKKTKE |
| | | 1123-1143 | LERIDLNLRDEYWLNFVQNNN |
| 4 | 101, 102, | 1-50 | -------------------------------------------------- |
| | 69, 212, | 51-100 | ---------------------X-----------XX------MEQALSN-- |
| | 255, 237, | 101-150 | -----LLYND--DAQWFKWYDXVRNYLTKKPQDDXKENKLKLNFDNXSLL |
| | 207, 216, | 151-200 | G--GWSDGQEKXKXAXLLKY-XNEXYLCILKTXN-------IFDTSKE-N |
| | 235, 227, | 201-250 | PIYXIXHSXASRLILRN-----LKFQTLAGKGFKGENG------------ |
| | 229, 70, | 251-300 | ---------IXYGXMGKXXPXKAIQCLQKXIK--------ERYVSKYPLL |
| | 105, 170 | 301-350 | EE----------FVXXKYTDKSXFDAXITETLKQCYVCEFEPIDWNLVXE |
| | | 351-400 | KXNNGELXLFXIHXKDYMPKX-----XXGKKXLXTXYWXDXXSE-GSKHQ |
| | | 401-450 | LCA-----GAEIFMRXPVAK----XX--XSKLVNKXDXDGNXXXX-XX-X |
| | | 451-500 | XX-XXXXX-X-XXXXXX-XXXXXXXXXXXXXXEIIKDKRFYGEXKXXFHC |
| | | 501-550 | PIKLNYEAKXY------XPKYAYPEVNXAIXESLQQSDXLQFIGIDRGEK |
| | | 551-600 | HLVYXXTVDKDXEIIXCXDXX----------------------------- |
| | | 601-650 | ---DNINGTDYVQXLXAVANERIIAXKNWQXIGKIKDLKSGYISXVVHRX |
| | | 651-700 | VEEVIKDGN-XXPXAXIVLEDLNTEXKRGRQ-KIEKXVYQNXEXALAKKL |
| | | 701-750 | NFVVDKDAXXXEXGSVXKALQLTPPIXN--YQDIEGKKQFGXMLYTRANY |
| | | 751-800 | TSVTDPATGWRKTIYXKNGKEEDIKKQILEKFSDFGFDGR-DYXFEYT-- |
| | | 801-850 | -----EAHAGX----TWRLYSGKNGKPLPRFXNKKQLQQDKNIWVPEQVN |
| | | 851-900 | VVEILDXLFAXFDKTKSFKXQIE--QGVELX-KIEXRXETAWQSLRXALD |
| | | 901-950 | LIQQIRNXGXEXX---------------DDXFLYSPVRNXXGEHFDTRNH |
| | | 951-1000 | ANNG-XXXXKDXDANGAYNIARKGLIMDXHIKYWXXXGKPX------KXN |
| | | 1001-1050 | DLDLFISDKEWDLWLLDRXXWEXELPXFASRNAKEXXDX----------- |
| 5 | 110, 113, | 1-50 | XXXXXXXXXXXXX-XXX-XXX-X-X-XXXXXXXXX---XX-XXXXX---- |
| | 111, 73, | 51-100 | XX--XXX-X---X-XXX-XX-XXXX-X----XX-X-----X-------X- |
| | 66, 54, 55, | 101-150 | --X--------X----------X------XX-XX--X-----X---X--- |
| | 112, 75, | 151-200 | ---XX--X---X----X--------------XX-X--------------- |
| | 106, 109, | 201-250 | --X--XXXX-X--X-XXX-XX--XX-X-XX-XXXXXXX-XXX--X-X--- |
| | 108, 53, | 251-300 | ---XX--X----XX--------X---X---X-XXX--X--X--XX--X-- |
| | 118, 100, | 301-350 | XX-----X------X----X-X-X-XX-X-XXXX------X--------- |
| | 103, 114, | 351-400 | ------X--X-------X----XXX-X--XX-XXXXXX----X-XXX--- |
| | 56, 67, | 401-450 | -X---X--X---------------X-X----XXXXX---X-AL------- |
| | 162 | 451-500 | -----X--X----X----XFYXXFLXXXXXX-EKXTXGYXXXKKXXEXLI |
| | | 501-550 | TXX-------------XKKDSX-KXXIKXFADXVLXIYQMXKYFALEKKX |
| | | 551-600 | XWX-XPYXLDXXFYXXFDEGXX--XXXDA-X-IVXYYNKXRNYLTKKP-Y |
| | | 601-650 | XXEKXKLNFENGTLLXGWDKNX----EKXX-XXILRKNERYYLGIIKXX- |
| | | 651-700 | NKXFXDE------XXQXYXDIINSGXXEXMVYKQLKDXTKXGX------- |
| | | 701-750 | ------X----X----------------------------FYXXD----- |
| | | 751-800 | ------XXXIXKXLXILXKKQXLXXYXSXQXYXEXX-X-XXX-X--X-XY |
| | | 801-850 | XSXKXFDFXXXXXXEXXX----XXX-X-X--XYKIXFXPXSEEYIXEKNX |
| | | 851-900 | XG-------ELYLFEIHNKDXXXX-----XXXKGTKNLHTLYFXXLFSQE |
| | | 901-950 | NLKHT-------FLKLXXG-AEXFYRXKXXEXKLXXXKXV-X-XX--X-- |
| | | 951-1000 | ---------XXXXXHKRXXEXKIXFHCPIXXN----XXESI--XKFNXKI |
| | | 1001-1050 | NNXLXNNXXXN--IIGXDRGEKXLAYYSVIXQKGXILXTXSLN------- |
| | | 1051-1100 | ------X--DINPVDYXXKLEXRXKERXXQRKXW--QXXXXIKDLKXGYI |
| | | 1101-1150 | SQVXXKXXXLXIX-----XNAIIVFEDLNMRFKXIRGGIEXXXYQQLEKA |
| | | 1151-1200 | LIXKLXXLVFKXXXDPEEXGXLLNXYQLTAPXXSFXXMGK--QTGXIFYX |
| | | 1201-1250 | XASYTSKXXP--XXGXRXNIYLKXEAIE--XAKLXITXFIXIXWDKDKXX |
| | | 1251-1300 | XXFSYXXKDFSEXKKXXXSKXXLYANAX------X-----XVERXXWDRR |
| | | 1301-1350 | YXX---XNXXXXL-XX-XX-------------DXTEXLXDLFXQIGIXYE |
| | | 1351-1400 | NG-XXGXXXXXEX----NENFXKXXIXXLNLIQQIRNXDSXRYX-X-X-- |
| | | 1401-1450 | ------------X---XXNXDFIAXPXXPFXSXXNPYTFXNXXX------ |
| | | 1451-1500 | -----------XXNGDANGAYNIARKGIXXLEXIKQXKXNP------XXX |
| | | 1501-1531 | XXX-XXDLYIXXXXWDKXXQKX---X----- |
| 6 | 104, 107, | 1-51 | --------------------------------------------------- |
| | 260, 253, | 52-102 | --------------------------------------------------- |
| | 91, 99, 92, | 103-153 | --------------------------------------------------- |
| | 262, 271 | 154-204 | --------------------------------------------------- |
| | | 205-255 | --------------------------------------------------- |
| | | 256-306 | --------------------------------------------------- |
| | | 307-357 | --------------------------------------------------- |
| | | 358-408 | --------------------------------------------------- |
| | | 409-459 | --------------------------------------------------- |
| | | 460-510 | --------------------------------------------------- |
| | | 511-561 | --------------------------------------------------- |
| | | 562-612 | --------------------------------------X------------ |
| | | 613-663 | -------X---X---X-X-X----X---XKXKXLFEIYXKDFSX------- |
| | | 664-714 | -----XXXXXXXXXXEXLFSXXNTEXXXFKLXXXAEVFFXEKXDX------ |
| | | 715-765 | --------------X-X-XKKXEKNXKXXXXIIKXXRXTEDXIXFHLPITL |
| | | 766-816 | NF--KGXXKXINXEVXXYIQENEXXXIIGXDXXXKXLXYYGXIDXRGNIYX |
| | | 817-867 | -EKTLXXXGXXXXXGX----KXXXTDYRXKLDIKXXERXXXRRXWTEIEGI |
| | | 868-918 | XDLKXGYXSFVXXEIAXLLVQNNXXIVXEELXGGFKKDR-KXIXKNXYQNL |
| | | 919-969 | XNXXXDKLSYLVXK-----X-KXILXXRNXLXLTPKXXXSEXXGNQXGXLF |
| | | 970-1020 | Y--XDAXXTS-KXDPVXGFXXX----LXLXXSNXKXXXXKXSXXXSIXFNE |
| | | 1021-1071 | EKXXXEFXXXXX--X--XXXKX----XXXXKXDXXXXXXXXEXXXSXXXXX |
| | | 1072-1122 | XXXXXYXXXXSLTEXFX-------------------------------KLF |
| | | 1123-1173 | QDY-XXX--XLXXXIVEXX-KXXXXFXXDXXXLXLXLRNXXXXX---XXDI |
| | | 1174-1224 | XXXXXXXXXXKLXFXX----XX---------X-XKDGDANGAYNIARKGLM |
| | | 1225-1275 | IXEKIXXXXXKXRXNXT------------IXSKXLYXXLQEWDKXXPXXXX |
| | | 1276-1326 | XX------------------------------------------------- |
| | | 1327-1354 | ---------------------------- |
| 7 | 269, 220, | 1-51 | ----XX-X-XXXXXXXX-XXXXXXXXXXXXXX-X-X-X--XXX------XX |
| | 225, 266, | 52-102 | XXXX-X-X-X-XXXX-XX-XXX-XXX-XXX-X--XX-XXXXX-------X- |
| | 186 | 103-153 | -XXX--XXXXXX--XXXX-X-XX-XX---------X-X-XXXXX--X--XX |
| | | 154-204 | X-------X-----X-X--XXXXXXXXX-XXXXXX-XXXXXXXXXXXXXXX |
| | | 205-255 | XXXXXX-XXX-X-XXXX------XX-X-X-XX-----XXXXXXX-X-XXX- |
| | | 256-306 | -XXX--XX-XXXXXX-X-XXXX--XXXXXXXXXXXXX-X--XXXX-X-XXX |
| | | 307-357 | XXXXXXX-XXXXXXXXX-X--XXX-X---X-XX-X--X----XX--X--XX |
| | | 358-408 | --X-XXXXXXXXXXXX-XX-XXXX--XX---------XXX---XXX-XXX- |
| | | 409-459 | -----X--X----X--XXXXX-XX-X--XXX-X-XXX--XXXXXXXXXX-- |
| | | 460-510 | ----X----XXXX---XX------X---X-XXXX--------XX--XX--- |
| | | 511-561 | X----X-XX-X--XX--XXX-X---XXXXXX--X-------XXXXXX-X-X |
| | | 562-612 | XXXXX---X--XXXXXXX-XXXXXX-XXXXXXX--XX-XXTGWVDSKTEKS |
| | | 613-663 | NXGTQFGGYLFRKKNEIGEYDYFLGISSKTXLFRKNEAXIG---XDYERLD |
| | | 664-714 | YYQPKANXIYGSAYEGENSYKEDKKXLNKVIIAXIEQIKXTNIKKSXIXXX |
| | | 715-765 | XXXXNISDDDKVTPSXXLXKIKKVSIDXYNGXLSXXSFQSVNKEVIGNLXK |
| | | 766-816 | TISCLKXKEXFHDLIKKDYQXFTEVQAXIDEXXXXXXXXYXXVSXXELXXX |
| | | 817-867 | XXDKXKPLXLFXIXNKDLXXAXXXXXXXXXKXRGXXNLHTMXFKALMSGXQ |
| | | 868-918 | XXXDXGSGXXFYRXXSXXXXKXTHPAXXXIXXXNXXXKDXXXXFXYDXXKX |
| | | 919-969 | RRXXEXKFXFHLSIXQNYXAXX-XXSXXXNXXXXXYXRXXXXXXIXGIDRG |
| | | 970-1020 | ERNLLYXSXIDXXGNIVXXXXXXXIXXXXNXXXXXXXXXXXXXXXXXTXYH |
| | | 1021-1071 | XLLDKREKXXXXNXXXWXXXXXIKXLKXGYXSQXXXXIXXLMXKYNAIIXX |
| | | 1072-1122 | EDLXXXFXXXXXXXXKXVYQXFEXXLXXKLXYLVXKXXPXXEXGGXXXAXQ |
| | | 1123-1173 | LAXXXTXXXXXXKQXGFXFYXPAWNTSXIDPVTGFXXLLXPKXXXXKXAXD |
| | | 1174-1224 | FFGXFXXIXXNXXXXXFEFXXXYXXFXXXXXXXXXRWTXCTXGXXXXXXXX |
| | | 1225-1275 | KXXXNXXXYXXXXXTXXXKXLFXXYXIDYXXGNXXXEXXXXXNXXFXXXXX |
| | | 1276-1326 | XXXXXXXXXXXXXXXXXXXXXXXXXXXXXXXXXXXXXXXXXXXXXXXXXXX |
| | | 1327-1365 | XXXXXXXXXXXXXXXXXXXXXXXXXXXXXXXXXXXXXXX |
| 8 | 194, 203, | 1-51 | --X--XX-XXXXXXXXXXXXXXXXXXXXXXX-------------------X |
| | 115, 211, | 52-102 | --XXX-XX-XXX-XXXXXXXXXXXXX-XXX--XX-----------XX-XXX |
| | 273, 249 | 103-153 | X--XX--XXXX--XX--X----XXXXXXXX-XXXX-X-X------------ |
| | | 154-204 | ---------X-X-XX-X-XX--X--X---XXXXXXX-X-XXXXXXXXXXXX |
| | | 205-255 | X-XXXXXXXXXXXXXXXXXXXXXXXXXXX-XXX------XX--X-XX-X-X |
| | | 256-306 | XX-XXX-X-XXXX-XX-XXXX--XXXXXXXXXX-XXXX---XXXXXXXXXX |
| | | 307-357 | XXXXXXXX--XXXX--XXXXXXXXXXXXXXXXXXXXXXXXXXXXXXXXX-X |
| | | 358-408 | X--XX--------X---XX-X-X-XXX-XXXXXXX-XXXX-XX--XXXXXX |
| | | 409-459 | -XXX-X--X--XX---XX-X--XX-----XXXX-XXXNRKKXXXSXXSXSI |
| | | 460-510 | XXIXEYXKEFYTXKXXTDEKSVIDXFXXXGXXENGTX-LFEXXEXAYXXXK |
| | | 511-561 | DXLNXXXXGNXRXEXXXDEXXXIKXLLDXLKKLQXFXXXXXGSGXESXXDX |
| | | 562-612 | AFYXXXXXLMEVXXEXXPLYNXVRNYLTQXPYSEXKXKXNFEXSXXXXGWD |
| | | 613-663 | XXXEXXXXXVIXXKDGLYXLAIXNKXXXXXLXXDXIKSXGXCYEKXXXXXI |
| | | 664-714 | XXXXXXXXXXXXXXXFXRXXXXXFXXAXXXXXXXXCXXXXXXXEGKXXXXX |
| | | 715-765 | XXXXX-XX-XXLXXLIDXXKXXFXXXEXXXXXYKDFGFKWXXXSEYXSXXE |
| | | 766-816 | FXNDIXRXXYXXKFXRXSXSYXNXLVKXGKIYLFXIYNKDFSPXSKGTPNX |
| | | 817-867 | HTXYXXALFSEXNLXXXXXXLNGXAEXFYRKXSLEXXAKXMXXXHHXXXXK |
| | | 868-918 | XXXN-XX--XX-YXIXKDXRYTEDKYFLHVPITLNFKAVXDNKXXIKVXEX |
| | | 919-969 | IKEXXI--EHXIGIDRGERNLLYXSXIDXKGNIVXQLSLNEIXNXXXX--E |
| | | 970-1020 | MXDYHAXLDKREXEXDEARXXWQTIEGIKNLKEGYXSQVVHIXSKXMXXYX |
| | | 1021-1071 | AIXVLEDLNXGFXRSRQKVEKQVYQKFEXMLIXKLNXLVDKKKPVSEA--- |
| | | 1072-1122 | -XGLXNAYQLXDEYKGFQKVGK-QXGFLFYVXAWNTSKIDPXTGFVXLXXA |
| | | 1123-1173 | KYENIDXAKKXXSXFDEIRYNXEKDXFEFVIXDYSKFXXKAXGTQXX-WTL |
| | | 1174-1224 | CXFGDRIVTXRXNNXXGXWXHKXVKLXXEFKNLFDQXNIDYTKS-XLKEXI |
| | | 1225-1275 | LSQNXA----XXXX----------XXLDLXGLMLQMRNSXP---XTXEDXX |
| | | 1276-1326 | XSPVXXDXGEFXDSRXXXXEDNLPIXADANGAYNIARKGLWXXXQIXXXDN |
| | | 1327-1357 | LEKVXLAXXXXXWLCXAQQKXXXXX------ |
| 9 | 132, 133, | 1-50 | -------FXXFTNLYPXSKTLRFELXPXGKTXENIEKXGILXXDEHRAES |
| | 124, 152, | 51-100 | YKKVKKIIDEYHKXFIDXXLXXFXLX----X-----XXLEEYXXLYX--X |
| | 151, 72, | 101-150 | KXEXXXKX-FXKXQXNLRKQIVXXLTKDXXYK--------------IXKK |
| | 206, 24, | 151-200 | ELIXEDLXXFVXX--------------------------XXXXLIXEFXD |
| | 25, 68, | 201-250 | FTTYFXGFHENRKNMYSAEEKSTAIAYRLIHENLPKFIDNMXXFXKIAX- |
| | 195, 232, | 251-300 | -XVAEXFXXIYXXXXEXXNX-----IXEMFXLDYXXXXLTQKQIXVYNAI |
| | 30, 12, | 301-350 | IGGXT-XX-XKKIKXINEYXNLYNQQQKD--XRLPKLKPLXKQILSDREA |
| | 182, 252, | 351-400 | XSWLPEEFXSDXEMLXAIXEXYXXLXX------------XKXLLXXLXXY |
| | 259, 222, | 401-450 | DLXGIXIXNDLQLT-DISQKXFGXWXVIXXAIKX--X--XP---XXKEXX |
| | 251, 190, | 451-500 | EXYEERISKXFKXXKSFSIXYXN-XXX-----------X--IEDYFATLG |
| | 209, 239, | 501-550 | --AVNTXXXQKENLFAQ-IENAYTDAXXLL--XXYPTXXNLSQDKXNVAK |
| | 250, 192, | 551-600 | IKALLDAXKDLQHFXKPLLGXGDEXXKDERFYGEXXXLWXELDX-XTPLY |
| | 205, 71, | 601-650 | NKVRNYXTRKPYSTEKIKLNFDNS--QLLGGWDXNKEXDNTSVILRKDGL |
| | 76, 215, | 651-700 | YYLXIMDKKSNXXXXX-XXXXXDG----------XCYEKMDYKL--LPGA |
| | 93, 264, | 701-750 | NKMLPK-------------------VFFSX-------------------- |
| | 208, 267, | 751-800 | ---SRIXEFXPSEXLLXX---YKXXT--------HKKGXXFXLXDCHXL- |
| | 183, 265, | 801-850 | --------XDFFKXSIXKHEDWX------XFGFKFSDT--XTYEDXSGFY |
| | 193, 210, | 851-900 | REVEQQGYKLSFXXVSVSYIDQLVXEGKLYLFQIYNKDFS---------E |
| | 89, 263, | 901-950 | YSKGTPNXHTLYWKMLFDERNLXXVVYKLNGQAEVFXRKXSIKX----PT |
| | 268, 270, | 951-1000 | HPANXPIKNKNXXNKKXEST--FEYDXIKDXRYTVDKFQFHVPITXNXKA |
| | 213, 224, | 1001-1050 | XGXX---NINPXVXXYXRXX-XDTHIIGIDRGERHLLYLSXIDLKGNIXX |
| | 218, 257, | 1051-1100 | QFXLNEIXNEYXGNTYKTNYHDLLXKXEGERXEARXSWQTIENIKELKEG |
| | 36, 178, | 1101-1150 | YLSQVXHKISKXMVXYNAIVVLEDLNMGFMRGRQKXEKQVYXKFEKMLID |
| | 187, 244 | 1151-1200 | KLNYXXDKXXDADEPGGLLHAYQLTNKFESFX--------XLGKQSGFLF |
| | | 1201-1250 | YIPAWNTSKIDPVTGFVNLXDTRYEX-VXKAXSFFSKFXSIRYNXEKXWF |
| | | 1251-1300 | EFXFD-YNDFTTKAEGTRTXWTLCTXGTRIETFRNPEKNXQWDXEEXNLT |
| | | 1301-1350 | DEFKXLFXKYGIDING--NLKEAIXX-------QTXXXFFXELLHLXKLX |
| | | 1351-1400 | LQMRNSITX---XXVDYLXSPVAXENGXFXDSRX-------XXLPENADA |
| | | 1401-1449 | NGAYNIARKGLWVIRXIXXXX-DXEKLKLA-IXNKEWLQFAQXKPYLND |
| 10 | 158, 230, | 1-50 | ----------XXKKIDXXTNXYXVSKTXRFXLIPVGKTXXNFXXKXXLEE |
| | 234, 140, | 51-100 | DEKXXEDYXKXKEIIDRYXRXXIXXVLSK--------XXLDXLKDYAXLY |
| | 164, 142, | 101-150 | YXXN-TDADXK-KXXECESKLRKEIXKXXKNXXEYNKLFNKKLIEXXLPX |
| | 141, 180, | 151-200 | XLKN----EDEKEVVASFKNFTTYFTGFFTNRKNMYSDEEKSTAIAYRCI |
| | 77, 78, | 201-250 | NENLPKFLDNVKAF--EKAXSKLXKXAIXXLXETXSGLCGTXLXDVFTVD |
| | 167, 13, | 251-300 | YFNFVLXQSGIDXYNXIIGGYTTSDGTKXKGXNEYINLYNQ---XVXKXX |
| | 35, 179 | 301-350 | KXPXLKXLYKQILSESEXVSFIPXKFEXDNELLSAVXEFYAN-X-----X |
| | | 351-400 | XLKKAIDETKLLFXNLDX--XSLNGIYXKNDXSVTNXSNSMFGSWSVIXD |
| | | 401-450 | LWNKXYDS--VNSNXXIKDIEKYEDKRKKAYKKXKSXSXSXXQVLISXS- |
| | | 451-500 | NXEX-XKSXVDYYKXSLXELXDXIXXKYXEAKXLFXXXYXNX--KXLKND |
| | | 501-550 | DKAXELIKNFLDSXKEXEKFIKPLSGTGKXXEKXELFYGEFTPLLDXXSX |
| | | 551-600 | IDSLYXKVRNYVT--XKPXSTDKIKLNFGNPQXLXGWDRNKEXDYXAVLL |
| | | 601-650 | XKDGKYYLAIXDKSNXKIXENIXXXDXX-SDCYEKIIYKLLPGPNKMLPK |
| | | 651-700 | VFFS--------------------XKXXXXFXPSDEILXIXKXGTFKK-- |
| | | 701-750 | ------------GDXFXXDDCHKLIDXYKESFKKXPX-------WSXXXF |
| | | 751-800 | KFKDTXEYNDISEFYNXVAXQGYKIXXXKIPTSXIDKLVXEGKXYLFQXY |
| | | 801-850 | NKDFSXXSKGTPNLHTXYFKMLFDERNLEXVVYKLNGXAEMFYRPASIKX |
| | | 851-900 | D-KIXHPXNXPIKN---KNPLNDKKXST----------FPYDXXKDXRXT |
| | | 901-950 | KDQFSLHXPITMNFK---APDXXXINDDVRXLLKSCXNNYXIGIDRGERN |
| | | 951-1000 | LXYXSVXDSNGXIXEQHSLNIIINEYXGKTYETXYHXXLDXXEKERDEXR |
| | | 1001-1050 | XNWKTIEXIKELKEGYISQXVHXICXLVVKYDAIIXMEDLNFGFKRGRTK |
| | | 1051-1100 | -XEKQVYQKFEKMLIDKLNYXVDKK----LDPEEEGGLLKAYQLTNKFES |
| | | 1101-1150 | FXKXGKQXGXIFYVPXWLTSKIDPXTGFXXLLYPKYE-XXDKAKDXISRF |
| | | 1151-1200 | DXIRYNA------XEDXFEFDIDYDKFPRTAXDYRKKWTXCTNGERIEXF |
| | | 1201-1250 | RNPXKNNEWDYXTXILTEXFKELFDNYSIXYXDX-DDXKXXILSXTKXKF |
| | | 1251-1300 | FEDXXKLLRLTLQMRNSXPG-----TDXDYXXSPVKXKNGNFYDSSKYXE |
| | | 1301-1350 | KXK----LPXDADANGAYNIARKGLWIIEQXKKXD--DVLXKEKLAISNX |
| | | 1351-1365 | EWLEXXQKNXX---- |
| 11 | 62, 121, | 1-50 | ----XSXFQEFXXXYXLSKTLRFELXPXGKTLEXIXAKG------XXLQD |
| | 61, 82, 4, | 51-100 | EKRAKDYXKVKQIXDXYHXDFIEEXLSX--XXXSEXLLQXXYDVYLK--- |
| | 29, 39, | 101-150 | ------KKSXXDD-LQKXXKXAQDXLXKXIVKXIXX--KGKXXFX--XXX |
| | 117, 58, | 151-200 | XXAKKXXKXDX--X-LXKXXKQQED-X--------X--X-DEALEIIKSF |
| | 57, 40, 27, | 201-250 | XXFXTYFTGFHENRKNMYSXEDXHTXIAYRXXXXNLPXFXXNLXIXESXK |
| | 7, 6, 31, 9, | 251-300 | XKXPELXYXEXIX-X--XXX---------X-NXXXFXLXEXFEXXXXNNX |
| | 28, 38, 37, | 301-350 | LXQSGITXXNTIIGGKXVNGXXXKXKGINEXIN-LYXQQIXDKS---XIA |
| | 26, 34, | 351-400 | KXXXLHKQILSDGEXVSFXXDKFXD------DSEVCQXVXEFYR------ |
| | 129, 96, | 401-450 | --XXX-HXETXFLXVQXLFA-XXDXDXXKIYVKNXKSLXXLSXQVFG--D |
| | 181, 168, | 451-500 | XSXXGXAXDXYYTXXVAPKFNDXXXKXKTDNXX-XX-XEKXXFKKGXHSL |
| | 47, 261, 2, | 501-550 | XTXEQAXEXYXXHHDDXX----XXX--XXXXYXKXNFAAIXX------XX |
| | 46, 22, 63, | 551-600 | XIXKIHNNLXXIKGFLEXEXXKXERQLXKEKX--------XKQXKELLDN |
| | 42, 44, 43, | 601-650 | TXNXLHFLKLXHXKXXXXX-TLLXKDEXFYGXFEELYDELAXIVPLYNKV |
| | 45, 20, 51, | 651-700 | RXYXXQKPXSXEKXKLNFXNXTLXNGWDLNKEK-DNXXXXXXKDXKYYLX |
| | 52, 64, 11, | 701-750 | XXXKXX--------------XKXFDNXPN--------------------- |
| | 84, 116, | 751-800 | -GKEXYXKXVYKLLPGXNKMLP---------------------------- |
| | 21, 14, | 801-850 | ------KVFFXXX------------------------------------- |
| | 119 | 851-900 | -------------------------------------------------- |
| | | 901-950 | ---------NXKYYNPSXX---------X--XXX----XX-XXXNX---- |
| | | 951-1000 | ----XPXXXKXX--DXFN--XXDCXKXIDFXKXSIXKHPEX-WX-XFGFX |
| | | 1001-1050 | FSXTSSYXXXXXFYREVENQ--GYKXXFXXIXXXYIDXLVXQGKLYLFQI |
| | | 1051-1100 | YNKDFSXXXXG--XPNLHTLYXKALFXERNLQXXXY------KLNGEAEX |
| | | 1101-1150 | FYRKXSXX-XXXXXHXAXEXXXNKNXDNPKKX------------------ |
| | | 1151-1200 | -------------------X-XX-X-X-YDXIKDKRXTXDKFXXHXPITM |
| | | 1201-1250 | NFXXXG-XXXXXFNXXXNXXXXXXAX-DVXXXXIDRGERHLXYXTXXXXK |
| | | 1251-1300 | GXIXXQXXXNXIGXDXMXX----XXXXXXYHXXLXXXEXXRXXARXXWXX |
| | | 1301-1350 | IXXIKEXKXGYLSXVVHXIXXLXXXYNAIVVXEDLNFGFKRGRFKVEKQX |
| | | 1351-1400 | YQXXEXXLIXKLNXLVXKDX-XXDXXGXXXXAXQLTXXFXXXKKXG--KQ |
| | | 1401-1450 | TGXXXYVPAXXTSKIXPVTGFVXXLX--PXYEXXXXSQXFFXKFDKICYN |
| | | 1451-1500 | XDKGYFEFSFD---------YXXFG-DKAXXXXXKWTIXSXGXXXXXXXX |
| | | 1501-1550 | XXXNXX-------------XDTRXXXXXXELXXLXKXYSIXYXXXX--XX |
| | | 1551-1600 | XXXXICXXXDKXFXXXLXXLLXTXLXXRXS-----XXXXXXDXXXSPVAX |
| | | 1601-1650 | XNGXFFXXX---------XXLAXXX----XPQXADANGAYHIXLKGLXLL |
| | | 1651-1700 | NRXKX---XXDXKKXXLXIXNXXXXXFXQNRX------------------ |
| | | 1701-1710 | ---------- |
| 12 | 219, 90 | 1-51 | XXXXXXXXXXXXLXXVSKTLRFELKPIGXTKEYXEXXXIXXXDXXXXEXXX |
| | | 52-102 | XVKXXXDXYHKXFIEEXLXDXXXXXXXXXLXXXXXLYXXXXXXXXXXKXFE |
| | | 103-153 | XIXXXLRKXISXXFKKXXXYXXLFXKEXIKNXLXXXXXXNXEXXXXXXXFX |
| | | 154-204 | DFTTYFTGXNQNRXNMYSXEXKXTAIAYRLIXXNLPXXXXNXKXFXXXXXX |
| | | 205-255 | XXXIKKQIXXXXNXXXXXYXXXXXXXXXXXXXXXXXXXXQXXIXXYNXXIX |
| | | 256-306 | GXXXXEXXKXQGXNEXXNLXXQFKXXXXPKXKXLXKQILSDXXSXSFXXXX |
| | | 307-357 | XXNDXEXXXSIXXXXXXXXXXXLXXXEXXXXXXXXNXXXYXLXXIXXXNDX |
| | | 358-408 | SLTXXSXXXYGDWXXXXXXXXXXXXXXYTGKXKKXXXKYXEXKXKXLKXIK |
| | | 409-459 | XXSIXXXDXLXXXXXXXXXXXXXXXEXXKNXXXXXXXXXXXXXXEXXXXXX |
| | | 460-510 | KXXXXXXNXLXKXXXXIXXIKXXLDSIKXXQXFXKXLXXXXXXXXXDXXFY |
| | | 511-561 | XXLXXXYXXLXXIXPLYNKVRNYXTXKPXSTEKXKLNFXXXTLLXGWDXNK |
| | | 562-612 | EXXNLGXXFXKDXXYXLGIXNXXNXKIXXXXXXXXXNXXYKKXXYKXXPGP |
| | | 613-663 | NKMLPKVFFSKSRXXEFXPSEEXLXXXXXXXXKKGXXFXXXXCHXLIXFFK |
| | | 664-714 | XSIXKHEXWSXFXFXFXXTXXYXDISXFYXEVEXQGYKITXXXIXXXYINX |
| | | 715-765 | LVDEGXLYLFQIYNKDFSXYSKGXPNLHTLYWXXXFXXXNLKDVXYKLNGX |
| | | 766-816 | AEIFYRKXSIXXXXXXXHPXNXPXXNKXPXNXKKXSXFXYDXXKDXRYXXD |
| | | 817-867 | KFQFHVPITMNFKAXGENXXNXXXXXXIXXXDXXHIIGIDRGERXLLYXXV |
| | | 868-918 | IXXXGXIVEQXXLNXXXXXXXXXXXHXXDYHELLDXXEKEXXXARXXWXTI |
| | | 919-969 | XXIKELKEGYXSQVXHXIXXLXXKYNAIVVLEDLNXGFKXXRXKXEKXVYX |
| | | 970-1020 | KFEXMLXXKLXYLVXKXXQXXXEXGXXLXAYQLXXXXXXXXXXGXQXGXXX |
| | | 1021-1071 | YXPAWXTSKIDPXTGFVNLFXTKXXXXEXXXXFXXKFXNIXXXXXXXXXXF |
| | | 1072-1122 | XFXYXXXXXXXXGXRXXWXXXSXGXRXXXFRNXXKNXEWDXXXVXLTXEFX |
| | | 1123-1173 | XLFXRYXXXXXXXXXXXXSXXXXXXXXAVXXXDXXXXFXXLFXLXVQXRNS |
| | | 1174-1224 | XXXXXXDXXXSPVKNXXXXFYXSXXXSXXLPXDADANGAYNIAXKGLXLVX |
| | | 1225-1255 | XIKXSXKXXXXKIXXXIXNXXWLXFXQEXXX |

TABLE 2
Conserved sequences identified by aligning
272 MAD7 GIG- nuclease homologs with clustalw.

| SEQ | | | Number of | |
|---|---|---|---|---|
| ID NO | Sequence | Cluster | sequences | Position |
| 815 | EEVXKDNNFYAELEEIYDELXP | 1 | 104 | 630-652 |
| 816 | SKNFDIKFCX | 1 | 104 | 891-901 |
| 817 | CIAKHPXWKNFGFKFS | 1 | 104 | 915-931 |
| 818 | EENLKXIVLKLNGEAELFXR | 1 | 104 | 1034-1054 |
| 819 | KSSIK | 1 | 104 | 1058-1063 |
| 820 | IXEQKSFNIVNG | 1 | 104 | 1217-1229 |
| 821 | YGFKRGRF | 1 | 104 | 1299-1307 |
| 822 | KVERQVYQKFETMLINKLNYLVFKDRX | 1 | 104 | 1308-1335 |
| 823 | MEQALSN | 4 | 14 | 92-99 |
| 824 | LLYND | 4 | 14 | 106-111 |
| 825 | DAQWFKWYDXVRNYLTKKPQDDXKENKL | 4 | 14 | 113-152 |
| | KLNFDNXSLLG | | | |
| 826 | IFDTSKE | 4 | 14 | 192-199 |
| 827 | LKFQTLAGKGFKGENG | 4 | 14 | 223-239 |
| 828 | ERYVSKYPLLEE | 4 | 14 | 291-303 |
| 829 | GSKHQLCA | 4 | 14 | 396-404 |
| 830 | GAEIFMRXPVAK | 4 | 14 | 409-421 |
| 831 | YQDIEGKKQFGXMLYTRANYTSVTDPAT | 4 | 14 | 731-791 |
| | GWRKTIYXKNGKEEDIKKQILEKFSDFG | | | |
| | FDGR | | | |
| 832 | TWRLYSGKNGKPLPRFXNKKQLQQDKNI | 4 | 14 | 816-874 |
| | WVPEQVNVVEILDXLFAXFDKTKSFKXQ | | | |
| | IE | | | |
| 833 | KLFQDY | 6 | 9 | 1120-1126 |
| 834 | XXTGWVDSKTEKSNXGTQFGGYLFRKKN | 7 | 5 | 600-654 |
| | EIGEYDYFLGISSKTXLFRKNEAXIG | | | |
| 835 | IEDYFATLG | 9 | 49 | 492-501 |
| 836 | XTPLYNKVRNYXTRKPYSTEKIKLNFDN | 9 | 49 | 596-625 |
| | S | | | |
| 837 | XCYEKMDYKL | 9 | 49 | 685-695 |
| 838 | LPGANKMLPK | 9 | 49 | 697-707 |
| 839 | XTYEDXSGFYREVEQQGYKLSFXXVSVS | 9 | 49 | 841-891 |
| | YIDQLVXEGKLYLFQIYNKDFS | | | |
| 840 | XLGKQSGFLFYIPAWNTSKIDPVTGFVN | 9 | 49 | 1191-1227 |
| | LXDTRYEX | | | |
| 841 | EDEKEVVASFKNFTTYFTGFFTNRKNMY | 10 | 14 | 159-215 |
| | SDEEKSTAIAYRCINENLPKFLDNVKAF | | | |
| 842 | SDCYEKIIYKLLPGPNKMLPKVFFS | 10 | 14 | 630-655 |
| 843 | KNPLNDKKXST | 10 | 14 | 868-879 |
| 844 | XEKQVYQKFEKMLIDKLNYXVDKK | 10 | 14 | 1052-1076 |

### 6.13.2. Example 2: Functional Analysis of Nucleic Acid-Guided Nucleases

### Methods for Testing In Vitro Function of GIG-Nucleases

The functional properties of novel GIG-nucleases are tested using an E. coli derived in vitro transcription-translations system previously described by Maxwell et al. (Methods. 2018 Jul. 1; 143:48-57). In brief, DNA sequences encoding the novel GIG-nuclease and the cognate guide RNA targeting a DNA sequence of choice are placed under the control of strong bacterial promoters and expressed in a cell-free system (available commercially from Arbor Biosciences, Ann Arbor, MI). Nuclease DNA sequences are amplified by PCR or synthesized de novo using Gibson Assembly, gene blocks, oligonucleotides, or similar methods. The nuclease DNA sequences are wild type or codon optimized. Specifically, transcription of the nuclease is driven by the T7 promoter (5′-TAATACGACTCACTATAG-3′), which is transcribed by T7 RNA polymerase expressed in the same reaction under the control of the constitutively active p70a promoter (e.g., plasmid pTXTL-P70a-T7rnap from Arbor Biosciences). Expression of the guide RNA is placed under the control of the P70a promoter and proper transcriptional termination is ensured by the presence of a strong transcriptional terminator. Alternatively, the template for gRNA transcription is omitted from the in vitro transcription-translation reaction and a synthetically synthesized gRNA is instead added after completed expression of the GIG-Cas nuclease. In addition, target DNA is added to the reaction either in the form of a circular plasmid or a linear DNA fragment. Expression of a functional nuclease and its cognate guide RNA will result in cleavage of the target DNA which can be detected by various analytical methods including mobility shift analysis on an agarose gel, by capillary electrophoresis or on microfluidic systems. Alternative readout methods include quantitative PCR.

For productive cleavage by CRISPR/Cas nucleases, a bona fide PAM (protospacer adjacent motif) sequence is required in the immediate vicinity of the protospacer target sequence. In the absence of a permissible PAM sequence, typically 3-5 nucleotides in length and positioned immediate adjacent to the protospacer sequence or a few nucleotides removed, no cleavage will occur. In the case of novel GIG-Cas nucleases for which the PAM sequence is originally unknown, the above described in vitro transcription-translation system is used, after modifications to the target sequence, to determine the recognized PAM sequences. For this purpose, a randomized stretch of nucleotides is introduced in a region immediately next to the protospacer sequence. Typically, such a region consists of 6, 7, 8, 9 or 10 randomized nucleotides. By subjecting the aforementioned library of randomized PAM sequences to GIG-nuclease digestion, sequences corresponding to permissible PAM nucleotide variants and locations are cleaved leaving sequences with non-conforming PAM variants undigested. By the means of high-throughput DNA sequencing (“next generation sequencing”, or NGS, manufactured by Illumina) the PAM profile is determined as the difference is abundance of each PAM sequence variant between a digested sample and a control devoid of a guide RNA or supplemented with an irrelevant guide RNA.

In certain instances where are a particular PAM sequence is prevalent for e.g., a specific subtype of nucleases, a screen based on nuclease-mediated cleavage and inactivation of a reporter gene is employed. For example, many members of the Cas12a family of proteins (Class II, Type V nucleases which includes MAD7) canonically recognize PAM sequences with a consensus motif of TTTV (where V=A, C or G) immediately 5′ of the protospacer sequence. In such cases a precursory nuclease activity screen as described by Maxwell et al. (Methods. 2018 Jul. 1; 143:48-57) and above can be designed to rapidly detect nuclease activity for a large number of novel nucleases. In one implementation, the reporter gene encodes a fluorescent protein, such as GFP or RFP. A PAM sequence motif corresponding to the tetranucleotide TTTA, TTTC or TTTG is identified within the coding region of the gene, preferably in proximity to ATG start codon, or immediately upstream of the open reading-frame and a guide-RNA is designed to facilitate cleavage of the target gene. Using the in vitro transcription-translation method of Maxwell et al, a novel GIG-nuclease, its cognate reporter-targeting guide RNA and the reporter protein are expressed in a test tube and the accumulation of reporter protein and the associated fluorescent signal is monitored over time (every 10 min for 18 hours). Cleavage of the reporter gene results in reduced fluorescence compared to a negative control lacking the target-specific guide RNA or supplemented with a non-targeting guide RNA, including guide RNAs with a scrambled spacer. Alternatively, the screen can be established in bacterial cells or any other cellular system, i.e., to implicitly test functionality in mammalian cells, using fluorescent reporter proteins or other commonly used reporters such as beta-galactosidase, luciferase or antibiotic selection markers. This system is suitable for screening hundreds of novel nucleases for activity and can be used as an initial screen of candidate nucleases when a presumptive PAM sequence is available. With the appropriate modification this system can also be used to assess relative activities and kinetic properties of nucleases.

In order to have a functioning targetable nuclease complex, a nucleic acid-guided nuclease and a compatible guide nucleic acid are needed. To determine the compatible guide nucleic acid sequence, specifically the scaffold sequence portion of the guide nucleic acid, multiple approaches are taken. First, scaffold sequences are looked for near the endogenous loci of each nucleic acid-guided nuclease. When no endogenous scaffold sequence is found, scaffold sequences found near the endogenous loci of the other novel GIG-Cas nucleases are tested.

A homology template is generated to assess the functionality of the nucleic acid-guided nucleases and corresponding guide nucleic acids. The homology template comprises a mutation relative to the target sequence. The mutations are flanked by regions of homology (homology arms or HA) which would allow recombination into the cleaved target sequence. Guide nucleic acids comprising various scaffold sequences are tested.

An expression construct encoding the nucleic acid-guided nuclease is added to host cells along with an editing polynucleotide as described above. Editing efficiency is determined by qPCR to measure the editing plasmid in the recovered cells in a high-throughput manner. The editing polynucleotide can comprise a selectable marker to allow easier selection of edited cells.

### Identification of Compatible PAM Sequences

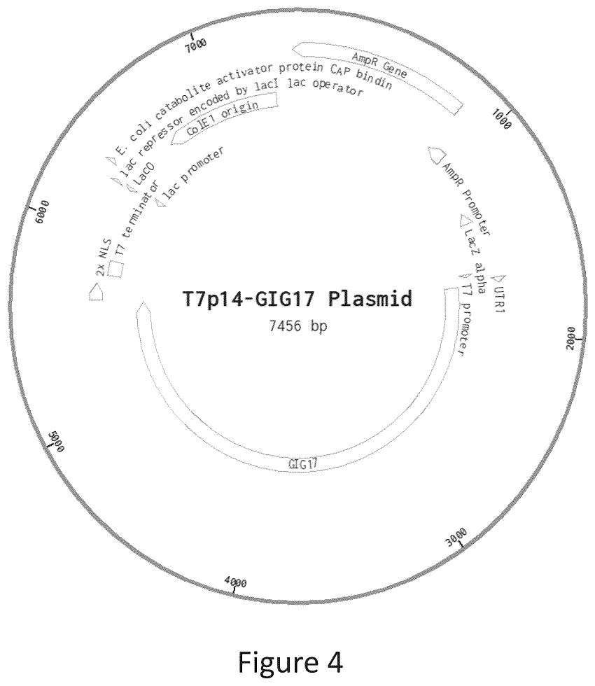

To elucidate the permissible PAM variants for novel Cas proteins the in vitro system of Maxwell et al. (Methods, 2018) was employed. Briefly, a plasmid containing the Cas protein ORF under the regulation of the T7 promoter (e.g., FIG. 4 and SEQ ID NO: 814), a plasmid containing a target gene, MR1 gene (NM_001531.2) with a randomized 10-mer cassette placed immediately 5′ of a protospacer (5′-sequence, and a synthetic DNA molecule encoding a cognate guide RNA (5′-gggcgtttcggatcccatccatgggg-3′) under the control of the P70a promoter were added to the in vitro transcription-translation system (Arbor Biosciences) and incubated for 18 h at 29° C. to allow expression of the nuclease and guide RNA and cleavage of the target DNA. Example guide RNAs are shown in Table 3 and example target MR1 sequences are shown in Table 4.

TABLE 3
Examples of guide RNAs for novel
GIG- nucleases targeting the MR1 gene.

| Cas protein | gRNA sequence |
|---|---|
| MAD7 | GTCAAAAGACCTTTTTAATTTCTACTCTTGTA |
| | GATgcctgggcgtttcggatccca |
| GCA_000156415.1_DS995364.1 | GTCTAAGAACTTTAAATAATTTCTACTGTTGT |
| | AGATgggcgtttcggatcccatccatggggtc |
| GCA_003436785.1_QSQP01000003.1 | GTTAAGTAATATAGAATAATTTCTACTGTTGT |
| | AGATgggcgtttcggatcccatccatgggg |
| GCA_002633275.1_NWBO01000064.1 | GTCTATAAGACGAACTAAATTTCTACTATTGT |
| | AGATgggcgtttcggatcccatccatggggtc |
| GCA_000988655.1_CP011377.1 | GTCTAACGACCTTTTAAATTTCTACTGTTTGT |
| | AGATgggcgtttcggatcccatccatggggt |
| GCA_002372885.1_DFJA01000037.1 | ATCTACAACAGTAGAAATTTAATTAGTAGGTC |
| | AAACgggcgtttcggatcccatccatgggg |
| GCA_902799665.1_CADCBK010000014.1 | ATCTACAACAGTAGAAATTTAGTATGAAGTTC |
| | AAACgggcgtttcggatcccatccatgggg |

Upper case = repeat sequence;
lower case = spacer sequence targeting the MR1 gene.

TABLE 4
Examples of target MR1
sequences with highlighted PAM sites.

| Target | |
|---|---|
| description | Target sequence |
| MR1 target | GACGCACTCTCTGAGATATTTTCgcctgggcgt |
| sequence | ttcggatcccaTCCATGGGGTC |
| for MAD7 | |
| MR1 target | GACGCACTCTCTGNNNNNNNNNNGCCTGGGCGT |
| sequence, | TTCGGATCCCATCCATGGGGTC |
| 5′ N₁₀ PAM | |
| MR1 target | GACGCACTCTCTGAGNNNNNNNNGCCTGGGCGT |
| sequence, | TTCGGATCCCATCCATGGGGTC |
| 5′ N₈ PAM | |
| MR1 target | GACGCACTCTCTGAGATNNNNNNGCCTGGGCGT |
| sequence, | TTCGGATCCCATCCATGGGGTC |
| 5′ N₆ PAM | |

Upper case, bold/underlined = PAM sequence, or randomized PAM screening cassette;
lower case = 21 base pair protospacer sequence for MAD7.

A negative control devoid of the guide RNA was run in parallel. Following the incubation, a DNA region encompassing the PAM cassette was PCR amplified and subjected to high-throughput sequencing. The nucleotide preference of the GIG-Cas nuclease for each position of the putative PAM cassette was computed as the relative difference in abundance between the guide RNA containing and deficient samples. Using this assay, the PAMs for GIG-1 (SEQ ID NO: 123), GIG-4 (SEQ ID NO: 254) and GIG-5 (SEQ ID NO: 28) were determined to be TTTV.

For higher throughput, a similar in vitro transcription-translation system was implemented in combination with a green fluorescent protein (GFP) reporter gene and evaluated the activity of 43 GIG-nucleases. The chosen 43 GIG-nucleases were representative of the protein sequence diversity of the full set (SEQ IDS 2-273) of GIG-Cas nucleases, i.e., the sequences analyzed represented a diverse sampling of the clades of GIG-Cas nucleases (i.e., FIG. 2). The reactions were essentially set up as described above, except in this instance the target gene encoded GFP and the guide RNA spacer sequence was chosen to reside in immediate proximity of a naturally occurring TTTC PAM sequence within the open-reading frame of the GFP protein. By cleaving the target gene close to the ATG start codon, reduced GFP activity was expected in a sample containing a target specific guide RNA compared to a control devoid of a guide RNA. In a successful experiment, a distinct reduction in fluorescence was observed when the reactions were supplemented with a GFP-targeting guide RNA, with some GIG-nucleases demonstrating a more pronounced effect than others. As described above for the MR1 targeting assay, following the incubation, a DNA region encompassing the PAM cassette was PCR amplified and subjected to high-throughput sequencing. The nucleotide preference of the GIG-Cas nuclease for each position of the putative PAM cassette was computed as the relative difference in abundance between the guide RNA containing and deficient samples.

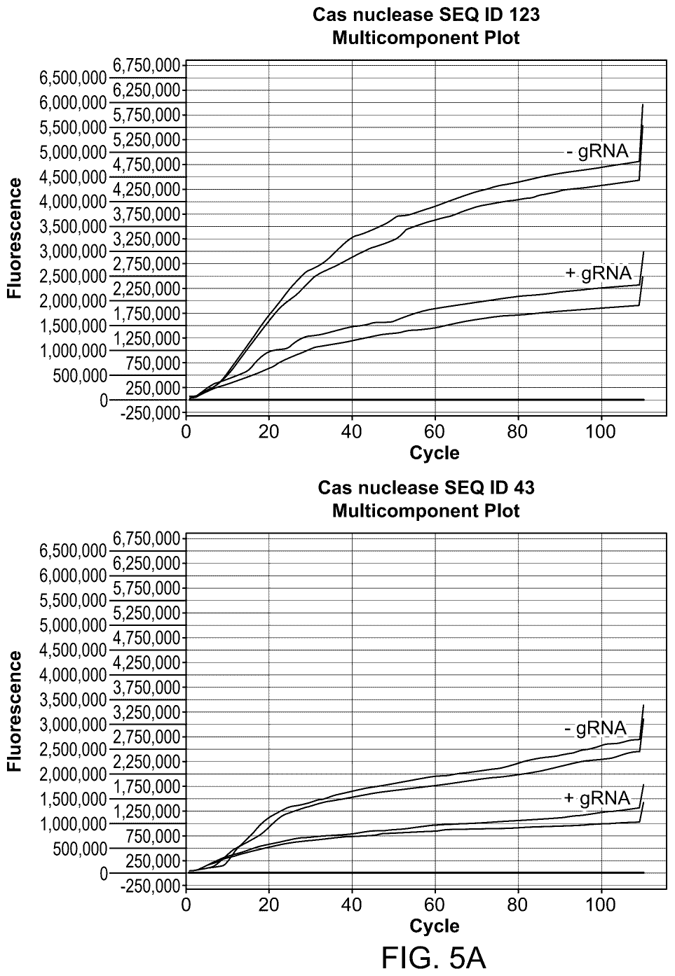

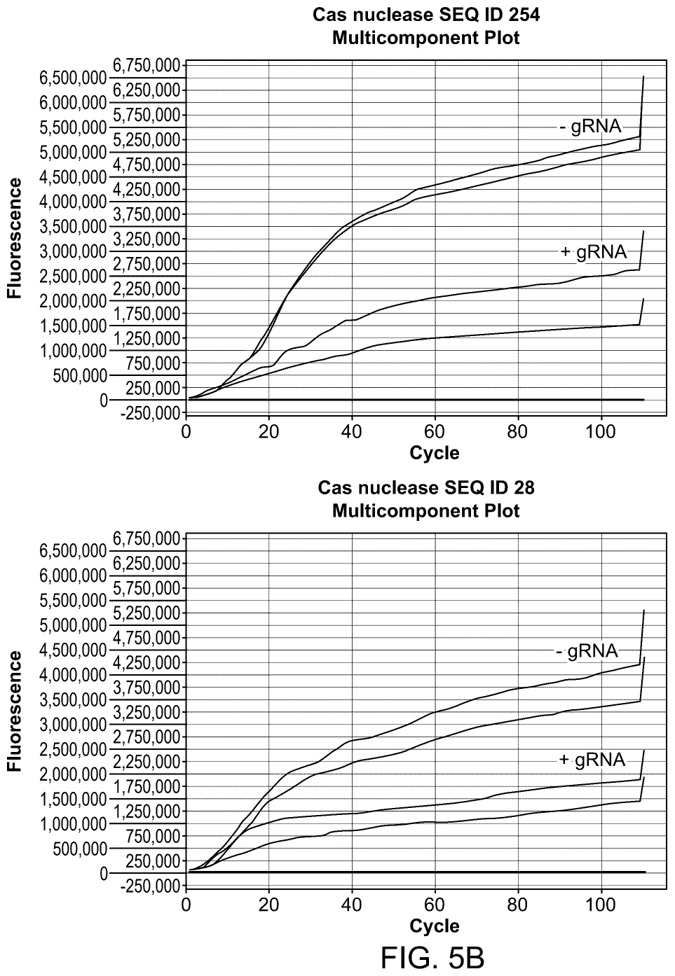

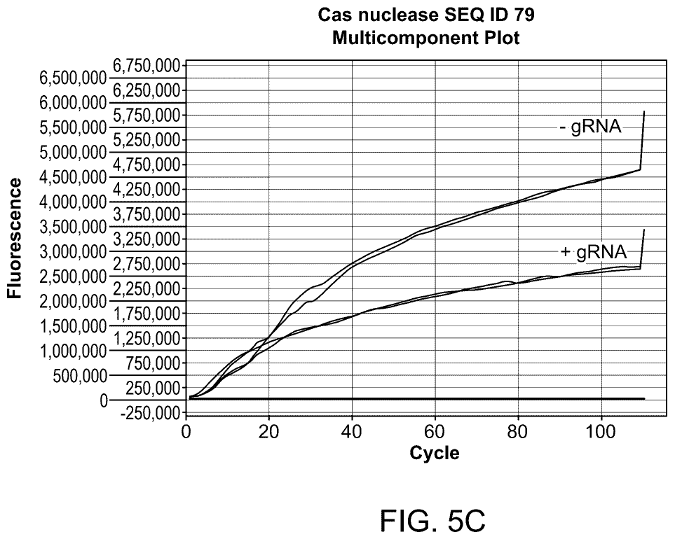

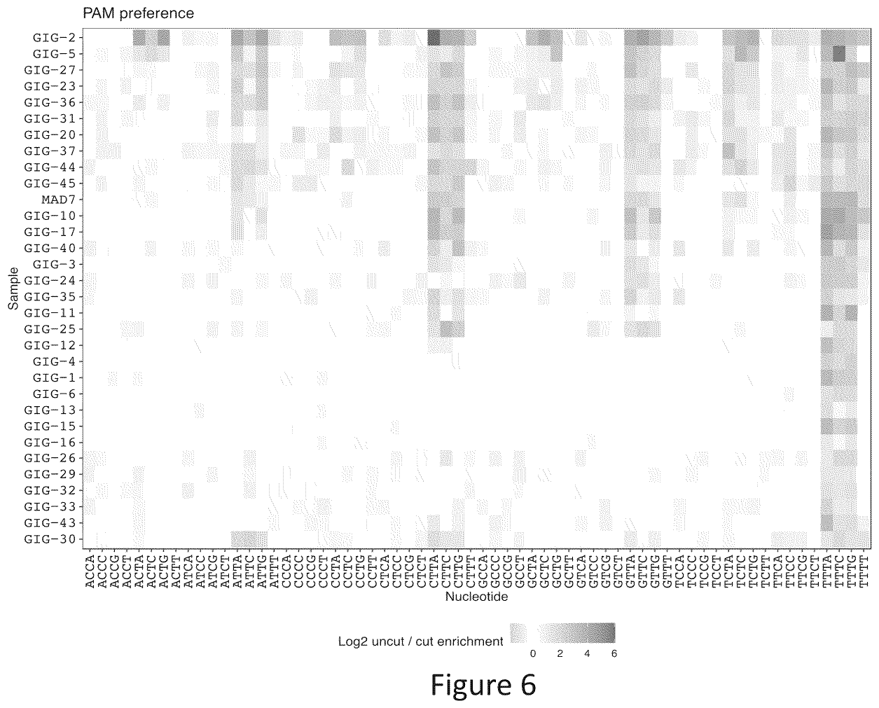

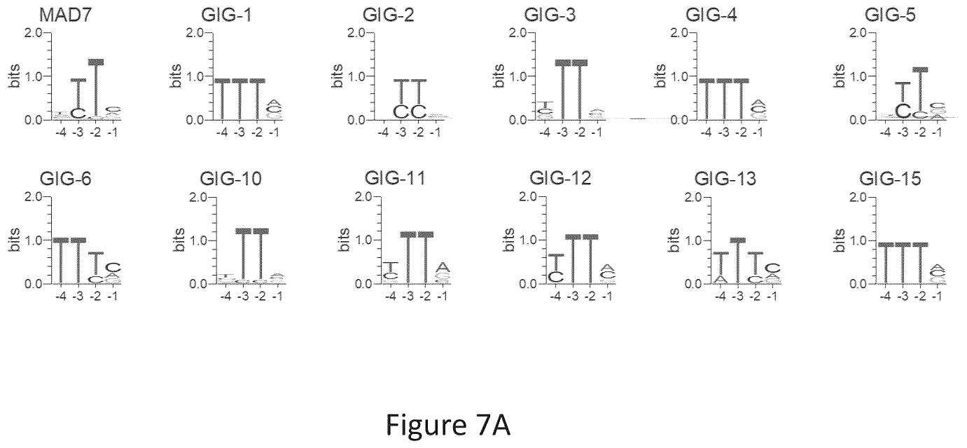

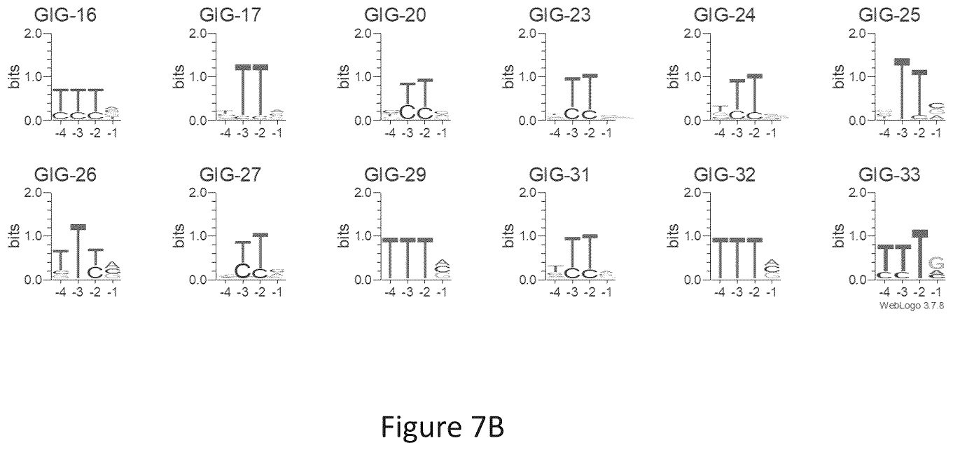

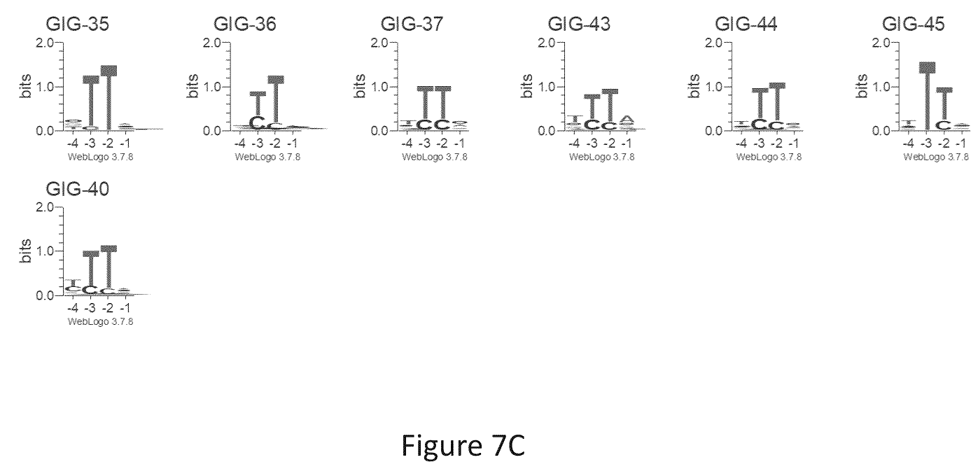

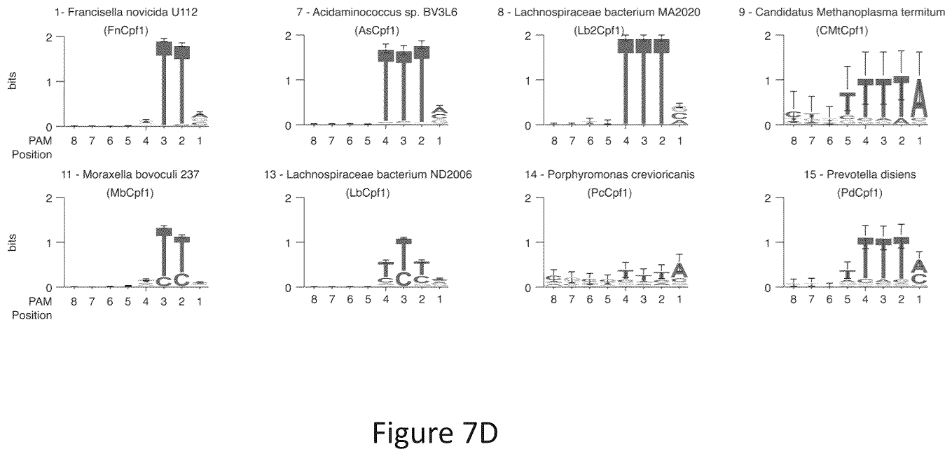

FIGS. 5A-5C show exemplary GFP reporter results of the PAM screen for GIG-1 (SEQ ID NO: 123), GIG-4 (SEQ ID NO: 254), GIG-3 (SEQ ID NO: 79), GIG-2 (SEQ ID NO: 43), and GIG-5 (SEQ ID NO: 28) from the present invention. FIG. 6 shows quantitative sequencing heatmap results for 31 example GIG-enzymes. FIGS. 7A-7D show sequence logos which summarize the heatmaps for 31 example GIG-enzymes. Table 5 provides the consensus, dominant PAM sequences identified for GIG-nucleases described herein. Though most GIG-nucleases of the present invention show similarities with previously disclosed Cpf1 nucleases, many of the GIG-nucleases show quantitative or qualitative differences from known PAM sequences. For example, GIG-2, GIG-20, and GIG-27 allow for cytosine nucleotides at the −3 and −2 positions of the PAM, in contrast with MAD7, which does not have strong activity with cytosine at the −2 position of the PAM. Such differences may confer advantages for genome engineering applications. Table 5 provides a look-up key to link enzyme ID, amino acid sequence, E. coli optimized nucleotide sequence, human optimized nucleotide sequence, protospacer adjacent motif (PAM), and cluster.

TABLE 5
Enzyme ID, Sequence, and Cluster

| | | E. coli | | | |
|---|---|---|---|---|---|
| | | optimized | Human optimized | | |
| Enzyme | Amino acid | nucleotide | nucleotide | | |
| ID | sequence | sequence | sequence | PAM | Cluster |
| GIG-1 | SEQ ID NO: 123 | SEQ ID NO: 632 | SEQ ID NO: 722 | TTTV | 1 |
| GIG-2 | SEQ ID NO: 43 | SEQ ID NO: 633 | SEQ ID NO: 723 | NTTN, | 11 |
| | | | | NCTV | |
| GIG-3 | SEQ ID NO: 79 | SEQ ID NO: 634 | SEQ ID NO: 724 | TTTV | 1 |
| | | | | (strongest), | |
| | | | | CTTV, | |
| | | | | GTTV | |
| GIG-4 | SEQ ID NO: 254 | SEQ ID NO: 635 | SEQ ID NO: 725 | TTTV | 1 |
| GIG-5 | SEQ ID NO: 28 | SEQ ID NO: 636 | SEQ ID NO: 726 | NTTN, | 11 |
| | | | | NCTV | |
| | | | | (weak) | |
| GIG-6 | SEQ ID NO: 98 | SEQ ID NO: 637 | SEQ ID NO: 727 | TTTV | 9 |
| GIG-7 | SEQ ID NO: 272 | SEQ ID NO: 638 | SEQ ID NO: 728 | Not found | 3 |
| GIG-8 | SEQ ID NO: 166 | SEQ ID NO: 639 | SEQ ID NO: 729 | Not found | 1 |
| GIG-9 | SEQ ID NO: 202 | SEQ ID NO: 640 | SEQ ID NO: 730 | Not found | 1 |
| GIG-10 | SEQ ID NO: 146 | SEQ ID NO: 641 | SEQ ID NO: 731 | TTTV | 1 |
| | | | | (strongest), | |
| | | | | NTTV, | |
| | | | | TCTV | |
| | | | | (weak) | |
| GIG-11 | SEQ ID NO: 175 | SEQ ID NO: 642 | SEQ ID NO: 732 | TTTV | 1 |
| | | | | (strongest), | |
| | | | | CTTV | |
| | | | | (weak) | |
| GIG-12 | SEQ ID NO: 197 | SEQ ID NO: 643 | SEQ ID NO: 733 | TTTV | 1 |
| | | | | (strongest), | |
| | | | | CTTV | |
| | | | | (weak) | |
| GIG-13 | SEQ ID NO: 214 | SEQ ID NO: 644 | SEQ ID NO: 734 | TTTV | 1 |
| GIG-14 | SEQ ID NO: 21 | SEQ ID NO: 645 | SEQ ID NO: 735 | Not found | 11 |
| GIG-15 | SEQ ID NO: 151 | SEQ ID NO: 646 | SEQ ID NO: 736 | TTTV | 9 |
| GIG-16 | SEQ ID NO: 142 | SEQ ID NO: 647 | SEQ ID NO: 737 | TTTV | 10 |
| GIG-17 | SEQ ID NO: 116 | SEQ ID NO: 648 | SEQ ID NO: 738 | TTTV | 11 |
| | | | | (strongest), | |
| | | | | NTTV, | |
| | | | | TCTV | |
| | | | | (weak) | |
| GIG-18 | SEQ ID NO: 103 | SEQ ID NO: 649 | SEQ ID NO: 739 | Not found | 5 |
| GIG-19 | SEQ ID NO: 55 | SEQ ID NO: 650 | SEQ ID NO: 740 | Not found | 5 |
| GIG-20 | SEQ ID NO: 172 | SEQ ID NO: 651 | SEQ ID NO: 741 | NTTV, | 1 |
| | | | | TCTV | |
| | | | | (weak) | |
| GIG-23 | SEQ ID NO: 210 | SEQ ID NO: 654 | SEQ ID NO: 744 | NTTV, | 9 |
| | | | | TCTV | |
| | | | | (weak) | |
| GIG-24 | SEQ ID NO: 232 | SEQ ID NO: 655 | SEQ ID NO: 745 | TTTV | 9 |
| | | | | (strongest), | |
| | | | | GTTV | |
| GIG-25 | SEQ ID NO: 83 | SEQ ID NO: 656 | SEQ ID NO: 746 | TTTV | 1 |
| | | | | (strongest), | |
| | | | | CTTV, | |
| | | | | GTTV | |
| GIG-26 | SEQ ID NO: 259 | SEQ ID NO: 657 | SEQ ID NO: 747 | TTTV | 9 |
| GIG-27 | SEQ ID NO: 179 | SEQ ID NO: 658 | SEQ ID NO: 748 | NTTV, | 10 |
| | | | | TCTV | |
| | | | | (weak) | |
| GIG-28 | SEQ ID NO: 188 | SEQ ID NO: 659 | SEQ ID NO: 749 | Not found | 1 |
| GIG-29 | SEQ ID NO: 252 | SEQ ID NO: 660 | SEQ ID NO: 750 | TTTV | 9 |
| GIG-30 | SEQ ID NO: 242 | SEQ ID NO: 661 | SEQ ID NO: 751 | NTTV | 1 |
| GIG-31 | SEQ ID NO: 77 | SEQ ID NO: 662 | SEQ ID NO: 752 | NTTV, | 10 |
| | | | | TCTV | |
| | | | | (weak) | |
| GIG-32 | SEQ ID NO: 217 | SEQ ID NO: 663 | SEQ ID NO: 753 | TTTV | 1 |
| GIG-33 | SEQ ID NO: 239 | SEQ ID NO: 664 | SEQ ID NO: 754 | TTTV | 9 |
| GIG-34 | SEQ ID NO: 106 | SEQ ID NO: 665 | SEQ ID NO: 755 | Not found | 5 |
| GIG-35 | SEQ ID NO: 191 | SEQ ID NO: 666 | SEQ ID NO: 756 | TTTV | 1 |
| | | | | (strongest), | |
| | | | | CTTV, | |
| | | | | GTTV | |
| GIG-36 | SEQ ID NO: 11 | SEQ ID NO: 667 | SEQ ID NO: 757 | NTTV, | 11 |
| | | | | TCTV | |
| | | | | (weak) | |
| GIG-37 | SEQ ID NO: 62 | SEQ ID NO: 668 | SEQ ID NO: 758 | NTTV, | 11 |
| | | | | TCTV | |
| | | | | (weak) | |
| GIG-38 | SEQ ID NO: 67 | SEQ ID NO: 669 | SEQ ID NO: 759 | Not found | 5 |
| GIG-39 | SEQ ID NO: 129 | SEQ ID NO: 670 | SEQ ID NO: 760 | Not found | 11 |
| GIG-40 | SEQ ID NO: 10 | SEQ ID NO: 671 | SEQ ID NO: 761 | TTTV | 1 |
| | | | | (strongest), | |
| | | | | CTTV, | |
| | | | | GTTV | |
| GIG-41 | SEQ ID NO: 131 | SEQ ID NO: 672 | SEQ ID NO: 762 | Not found | 1 |
| GIG-42 | SEQ ID NO: 84 | SEQ ID NO: 673 | SEQ ID NO: 763 | Not found | 11 |
| GIG-43 | SEQ ID NO: 158 | SEQ ID NO: 674 | SEQ ID NO: 764 | TTTV | 10 |
| GIG-44 | SEQ ID NO: 200 | SEQ ID NO: 675 | SEQ ID NO: 765 | NTTV, | 1 |
| | | | | TCTV | |
| | | | | (weak) | |
| GIG-45 | SEQ ID NO: 82 | SEQ ID NO: 676 | SEQ ID NO: 766 | NTTV | 11 |

### Expression and Purification of Nuclease Proteins

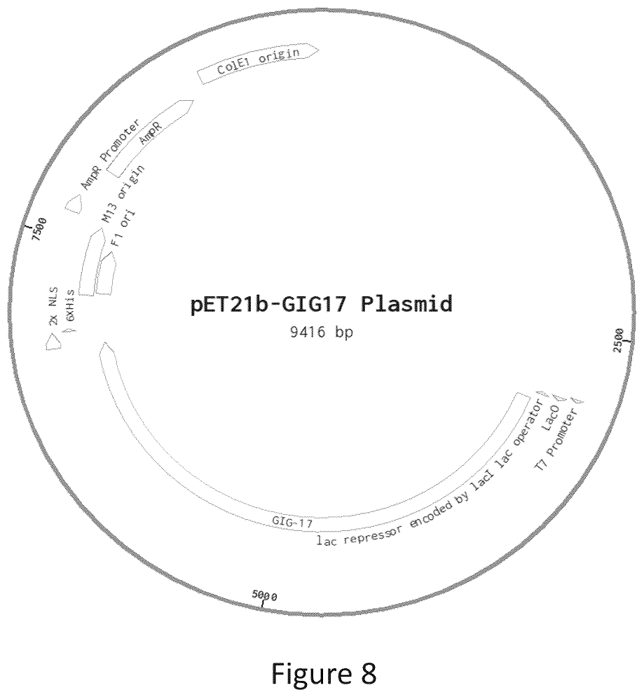

The nucleases of the present invention are purified using methods well known by those skilled in the art. Coding sequences of the nucleases were codon-optimized for E. coli (e.g., SEQ ID Nos: 632-676 and 677-721) and cloned in to a pET21b expression vector (e.g., FIG. 8 and SEQ ID NO: 812 for pET21b-GIG-17) in frame with a 6×his tag. Other types of purification tags can be also used, e.g., FLAG tag, etc. The plasmid was transformed into Rosetta2 (DE3) E. coli, which were cultured to an OD of 0.5, placed on ice for 15 minutes, then induced with 1 mM IPTG and shaken overnight at 20 C for expression. Cells were harvested and lysed by chemical and/or physical methods. His-tagged protein was captured from the lysate using free IMAC resin (Ni-NTA), or resin packed in a column, with imidazole for elution. Further purification was performed using CEX column chromatography at pH 5.5-7.5 and high salt elution. Final polishing was performed using size exclusion chromatography. Purified nucleases are formulated in 20 mM HEPES, 500 mM NaCl pH 7.5, and stored at 4 C or −80 C.

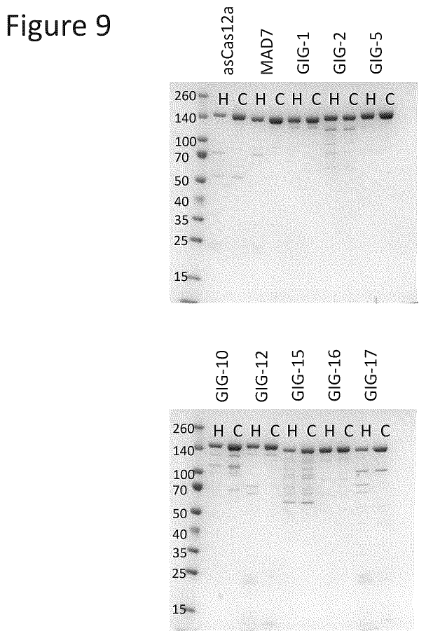

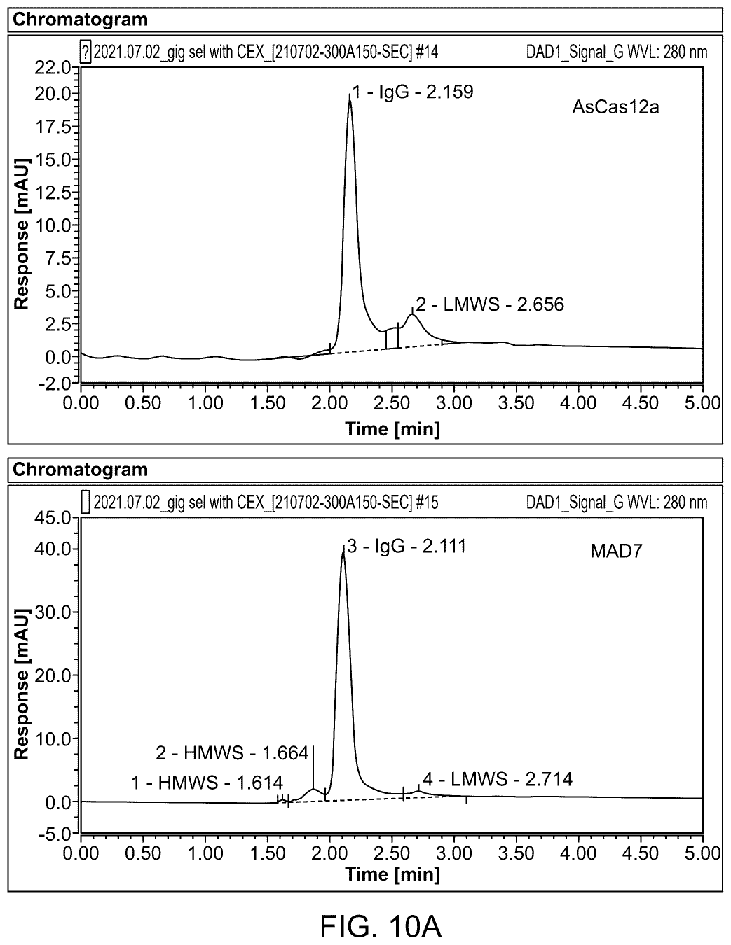

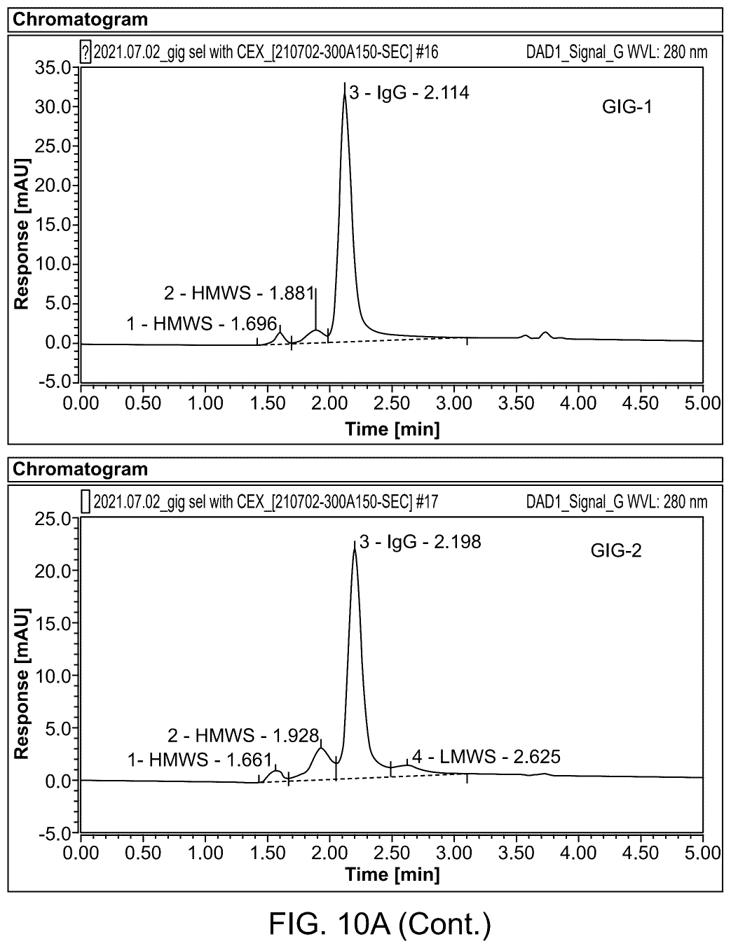

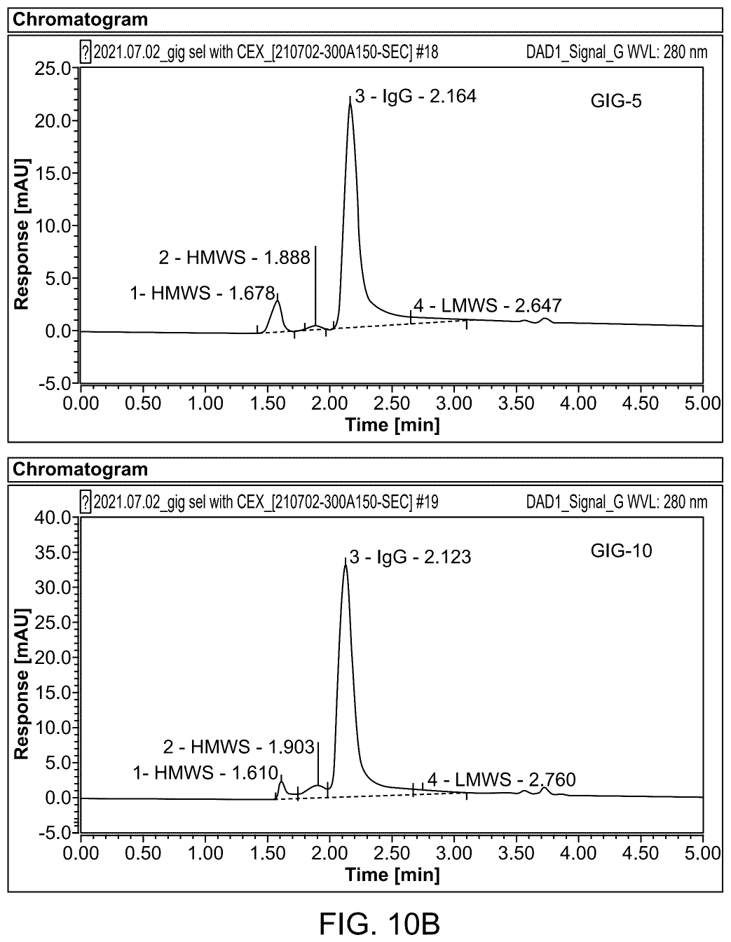

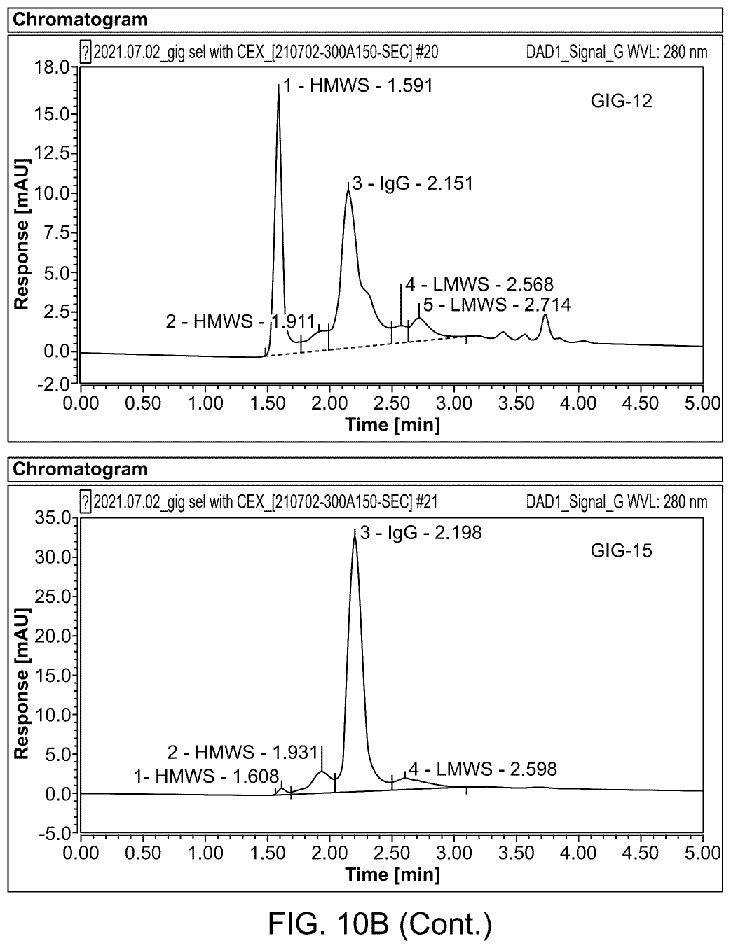

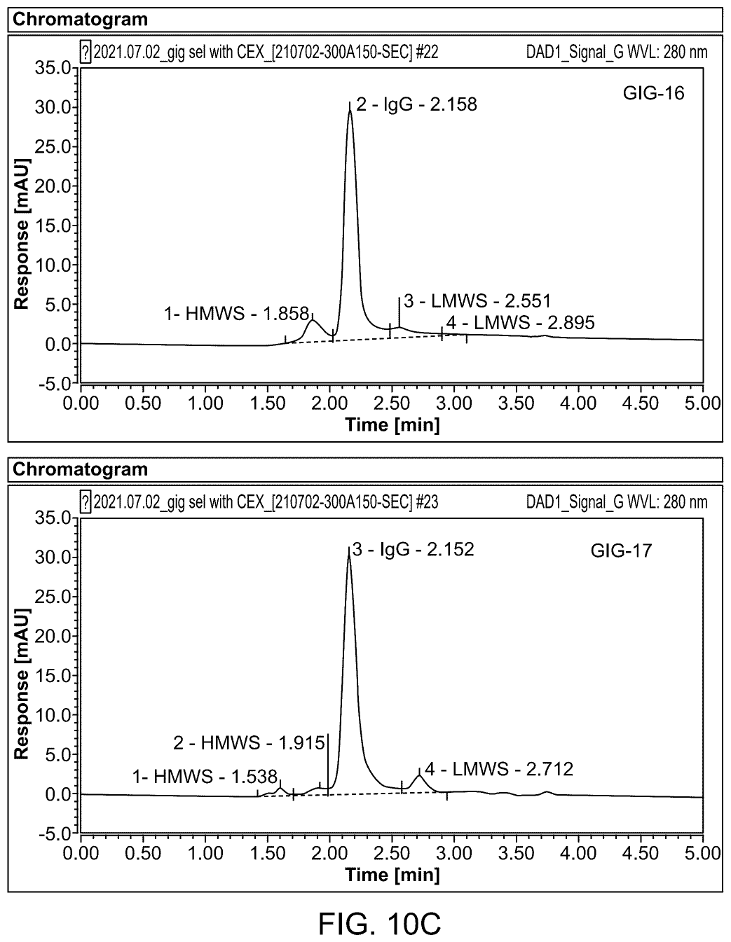

Purified protein was assessed for purity using SDS-PAGE (FIG. 9) and SE-HPLC (FIGS. 10A-10C), with concentration by A280. Results are summarized in Table 6.

TABLE 6
Purification results for GIG- nucleases of the present invention
using Ni-NTA (His-tag capture) and CEX purification methods.

| | After Ni-NTA | | | After CEX | | | |
|---|---|---|---|---|---|---|---|
| | micrograms | Gel purity | SEC purity | micrograms | Gel purity | SEC purity | Mg/mL |
| AsCas12a | 788 | 67% | 38% | 157 | 90% | 84% | 5.22 |
| MAD7 | 723 | 84% | 83% | 257 | 99% | 91% | 10.29 |
| GIG-1 | 3087 | 96% | 90% | 1179 | 96% | 91% | 23.58 |
| GIG-2 | 3089 | 76% | 86% | 1514 | 81% | 75% | 30.27 |
| GIG-5 | 1888 | 100% | 86% | 838 | 100% | 87% | 20.95 |
| GIG-10 | 1967 | 79% | 88% | 779 | 68% | 88% | 12.98 |
| GIG-12 | 444 | 64% | 25% | 78 | 87% | 52% | 1.96 |
| GIG-15 | 1357 | 63% | 77% | 467 | 64% | 82% | 10.38 |
| GIG-16 | 2709 | 93% | 86% | 1537 | 92% | 81% | 23.64 |
| GIG-17 | 401 | 50% | 57% | 78 | 73% | 88% | 3.1 |

### Genome Editing Activity Assay for GIG-Nucleases

SpCas9 (Synthego Corporation, Redwood City, CA, USA), Alt-R AsCas12a (Cpf1) V3 (IDT, Coralville, IA, USA), purified MAD7 and purified GIG-nucleases were electroporated into Jurkat E6-1 cells (TIB-152, ATCC, Manassas, VA, USA) using the Amaxa Nucleofector system (Lonza, Basel, Switzerland). Ribonucleoprotein (RNP) complexes were prepared by incubating SpCas9, AsCas12a, or GIG-nucleases with synthetic guide RNA (sgRNA) at a 1:1.2 molar ratio for 10 minutes at room temperature. SgRNA sequences used with asCas 12a and GIG-nucleases were synthesized by IDT (Coralville, IA, USA) and (are provided in Table 7. The sgRNA sequence used with spCas9 was synthesized by Synthego Corporation (Redwood City, CA, USA) and consists of a TRAC-targeting protospacer (Table 7) and a proprietary scaffold from Synthego. Cells were pelleted and resuspended in Nucleofection Buffer SE (Lonza, Basel, Switzerland) at 1×10⁷ cells/mL. Alt-RR Cpf1 Electroporation Enhancer (IDT, Coralville, IA, USA) was added to the cells, then 20 μL of the cell suspension was mixed with 40 pmol RNP complex immediately before electroporation. Cells were then transferred to a 96-well plate, resuspended in 200 μL RPMI medium supplemented with 10% FBS. After recovering for 24 hours, the cells were transferred to 6-well plates containing 2 mL RPMI medium supplemented with 10% FBS. Cells were analyzed for knockdown efficiency by flow cytometry 5 days after electroporation.

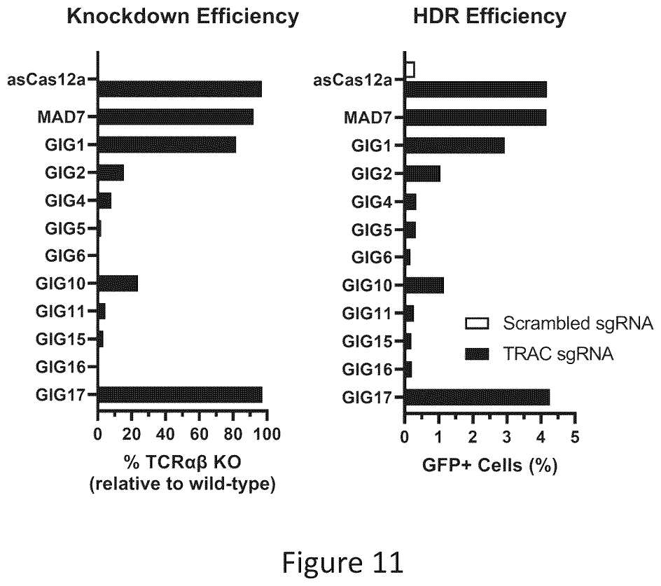

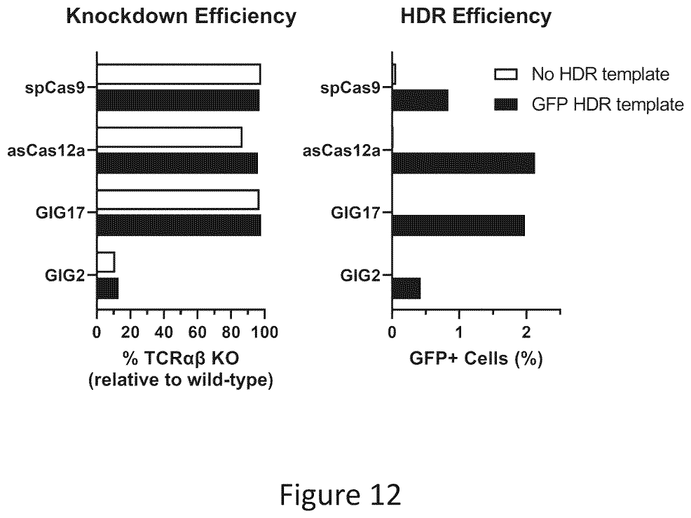

Electroporated Jurkat E6-1 cells were washed with MACS buffer, then stained with APC anti-human CD3 antibody (Clone UCHT1, BioLegend, San Diego, CA, USA) and PerCP/Cyanine5.5 anti-human TCR α/β Antibody (Clone IP26, BioLegend, San Diego, CA, USA) for 30 minutes at 4° C. After washing twice with MACS buffer, cells were stained with DAPI and analyzed using a CytoFLEX flow cytometer (Beckman Coulter, Brea, CA, USA) and FlowJo software (BD Biosciences, San Jose, CA, USA). Cytometry data for 50,000 live (DAPI-) cells was collected for each sample. TCR knockdown efficiency was determined by assessing the percentage of TCRαβ+/CD3+ cells in electroporated samples normalized to wild-type cells. The results of these experiments are shown in Tables 8-9 and FIGS. 11-12.

TABLE 7
TRAC target genome and sgRNA sequences.

| | sgRNA Target | |
|---|---|---|
| sgRNA | (Gene: (PAM) | |
| Name | gRNA Sequence) | sgRNA Sequence |
| GR-31 | TRAC: | Protospacer: |
| | AGAGTCTCTCAGCT | rArGrA rGrUrC rUrCrU rCrArG |
| | GGTACA(CGG) | rCrUrG rGrUrA rCrA |
| GR-40 | Scrambled/non- | rUrA rArUrU rUrCrU rArCrU |
| | targeting: | rCrUrU rGrUrA rGrArU rCrGrU |
| | CGTTAATCGCGTAT | rUrArA rUrCrG rCrGrU rArUrA |
| | AATACGG | rArUrA rCrGrG |
| GR-42 | TRAC: | rUrA rArUrU rUrCrU rArCrU |
| | (TTTA)GAGTCTCT | rCrUrU rGrUrA rGrArU rGrArG |
| | CAGCTGGTACACGG | rUrCrU rCrUrC rArGrC rUrGrG |
| | C | rUrArC rArCrG rGrC |

TABLE 8
Knockdown and HDR Efficiency of selected GIG nucleases
at the human TRAC locus in Jurkat cells.

| | | TCR Knockdown | HDR Efficiency |
|---|---|---|---|
| Nuclease | sgRNA Target | Efficiency | (GFP + cells) |
| asCas12a | Scrambled (GR-40) | 0.0% | 0.31% |
| asCas12a | TRAC (GR-42) | 97.1% | 4.18% |
| MAD7 | TRAC (GR-42) | 92.2% | 4.17% |
| GIG-1 | TRAC (GR-42) | 81.9% | 2.94% |
| GIG-2 | TRAC (GR-42) | 15.5% | 1.05% |
| GIG-4 | TRAC (GR-42) | 8.1% | 0.35% |
| GIG-5 | TRAC (GR-42) | 2.1% | 0.33% |
| GIG-6 | TRAC (GR-42) | 0.8% | 0.18% |
| GIG-10 | TRAC (GR-42) | 23.9% | 1.16% |
| GIG-11 | TRAC (GR-42) | 4.5% | 0.28% |
| GIG-15 | TRAC (GR-42) | 3.4% | 0.20% |
| GIG-16 | TRAC (GR-42) | 1.1% | 0.22% |
| GIG-17 | TRAC (GR-42) | 97.4% | 4.27% |

TABLE 9
Knockdown and HDR Efficiency of selected GIG nucleases at
the human TRAC locus in Jurkat cells.

| | | TCR Knockdown | | HDR Efficiency | |
|---|---|---|---|---|---|
| | | Efficiency | | (GFP + cells) | |
| | | No | GFP | No | GFP |
| Nuclease | sgRNA Target | Template | Template | Template | Template |
| spCas9 | TRAC (GR-31) | 97.9% | 97.2% | 0.059% | 0.84% |
| asCas12a | TRAC (GR-42) | 86.6% | 96.0% | 0.021% | 2.13% |
| GIG-17 | TRAC (GR-42) | 97.0% | 97.8% | 0.013% | 1.98% |
| GIG-2 | TRAC (GR-42) | 11.1% | 12.9% | 0.011% | 0.43% |

The results show that some nucleases in cluster 1 or 11 (e.g., GIG-1, GIG-17, GIG-10, GIG-2) have particularly strong nuclease activity compared to other nucleases in other clusters, as summarized in Table 10.

TABLE 10
GIG nucleases in the order of HDR Efficiency

| | | HDR | | | TCR |
|---|---|---|---|---|---|
| | | Efficiency | | | Knockdown |
| Nuclease | Cluster | (GFP + cells) | Nuclease | Cluster | Efficiency |
| GIG-17 | 11 | 4.27% | GIG-17 | 11 | 97.40% |
| (SEQ ID NO: 116) | | | (SEQ ID NO: 116) | | |
| GIG-1 | 1 | 2.94% | GIG-1 | 1 | 81.90% |
| (SEQ ID NO: 123) | | | (SEQ ID NO: 123) | | |
| GIG-10 | 1 | 1.16% | GIG-10 | 1 | 23.90% |
| (SEQ ID NO: 146) | | | (SEQ ID NO: 146) | | |
| GIG-2 | 11 | 1.05% | GIG-2 | 11 | 15.50% |
| (SEQ ID NO: 43) | | | (SEQ ID NO: 43) | | |
| GIG-4 | 1 | 0.35% | GIG-4 | 1 | 8.10% |
| (SEQ ID NO: 254) | | | (SEQ ID NO: 254) | | |
| GIG-5 | 11 | 0.33% | GIG-11 | 1 | 4.50% |
| (SEQ ID NO: 28) | | | (SEQ ID NO: 175) | | |
| GIG-11 | 1 | 0.28% | GIG-15 | 9 | 3.40% |
| (SEQ ID NO: 175) | | | (SEQ ID NO: 151) | | |
| GIG-16 | 10 | 0.22% | GIG-5 | 11 | 2.10% |
| (SEQ ID NO: 142) | | | (SEQ ID NO: 28) | | |
| GIG-15 | 9 | 0.20% | GIG-16 | 10 | 1.10% |
| (SEQ ID NO: 151) | | | (SEQ ID NO: 142) | | |
| GIG-6 | 9 | 0.18% | GIG-6 | 9 | 0.80% |
| (SEQ ID NO: 98) | | | (SEQ ID NO: 98) | | |

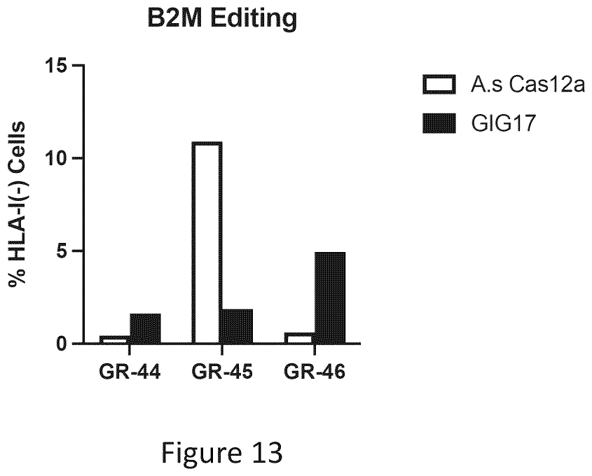

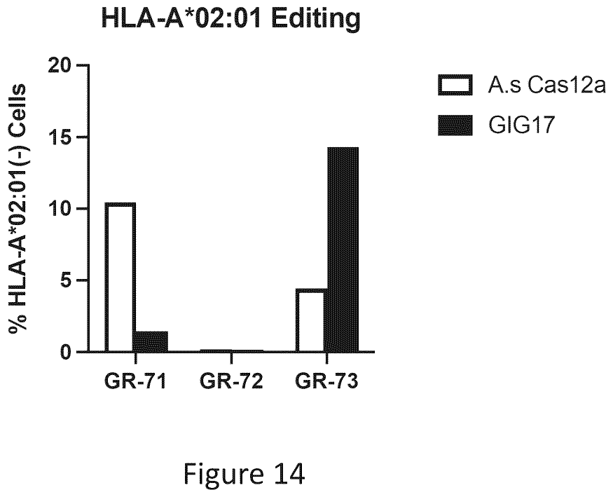

Genome editing activity of purified GIG-17 was further analyzed and compared to that of Alt-R AsCas12a (Cpf1) V3 (IDT, Coralville, IA, USA). RNPs were generated as described above with sgRNAs (Table 11) designed to generate loss-of-function mutations within the B2M and HLA-A*02:01 genes in Jurkat E6-1 and T2 cell lines, respectively. Jurkat E6-1 cells were resuspended in Nucleofection Buffer SE and T2 cells were resuspended in Nucleofection Buffer SF (Lonza, Basel, Switzerland) at 1×10⁷ cells/mL. 20 μL of the cell suspension was mixed with 40 pmol RNP complex immediately before electroporation. Cells were then transferred to a 96-well plate, resuspended in 200 μL of appropriate medium (RPMI medium supplemented with 10% FBS for Jurkat E6-1 cells and IMDM medium supplemented with 20% FBS for T2 cells). After recovering, the cells were transferred to 6-well plates containing 2 mL of appropriate medium. Cells were analyzed for knockdown efficiency by flow cytometry 5 days after electroporation as described above. Jurkat E6-1 cells were stained with PE anti-human HLA-A,B,C antibody (clone W6/32, BioLegend), and T2 cells were stained with PE anti-human HLA-A2 antibody (clone BB7.2, BioLegend). Knockdown efficiency was determined by assessing the percentage of HLA-deficient cells in the electroporated samples. The results of these experiments are shown in Tables 12-13 and FIGS. 13-14.

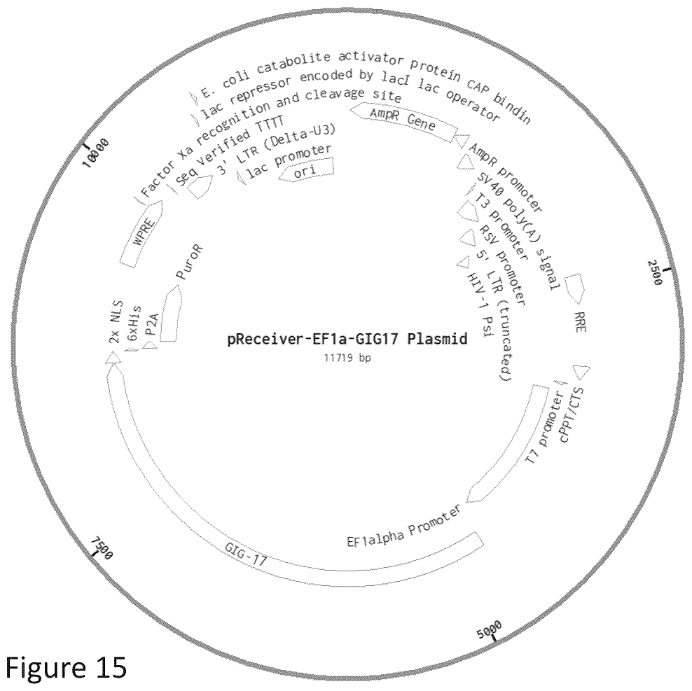

An example GIG-nuclease mammalian expression vector is shown in FIG. 15 and SEQ ID 813. Example GIG-nucleases codon optimized for mammalian expression are listed in SEQ ID 722-811.

TABLE 11
B2M and HLA-A*02:01
target genome and sgRNA sequences.

| | sgRNA Target | |
|---|---|---|
| sgRNA | (Gene: (PAM) | |
| Name | gRNA Sequence) | sgRNA Sequence |
| GR-44 | B2M: | rUrA rArUrU rUrCrU rArCrU |
| | (TTTC)ATCCATCC | rCrUrU rGrUrA rGrArU rArUrC |
| | GACATTGAAGTTGA | rCrArU rCrCrG rArCrA rUrUrG |
| | C | rArArG rUrUrG rArC |
| GR-45 | B2M: | rUrA rArUrU rUrCrU rArCrU |
| | (TTTC)CCGATATT | rCrUrU rGrUrA rGrArU rCrCrG |
| | CCTCAGGTACTCCA | rArUrA rUrUrC rCrUrC rArGrG |
| | A | rUrArC rUrCrC rArA |
| GR-46 | B2M: | rUrA rArUrU rUrCrU rArCrU |
| | (TTTA)CTCACGTC | rCrUrU rGrUrA rGrArU rCrUrC |
| | ATCCAGCAGAGAAT | rArCrG rUrCrA rUrCrC rArGrC |
| | G | rArGrA rGrArA rUrG |
| GR-71 | HLA-A*02:01: | rUrA rArUrU rUrCrU rArCrU |
| | (TTTC)CCTCCCGA | rCrUrU rGrUrA rGrArU rCrCrU |
| | CCCCGCACTCACCC | rCrCrC rGrArC rCrCrC rGrCrA |
| | GC | rCrUrC rArCrC rCrGrC |
| GR-72 | HLA-A*02:01: | rUrA rArUrU rUrCrU rArCrU |
| | (TTTC)TTCACATC | rCrUrU rGrUrA rGrArU rUrUrC |
| | CGTGTCCCGGCCCG | rArCrA rUrCrC rGrUrG rUrCrC |
| | GC | rCrGrG rCrCrC rGrGrC |
| GR-73 | HLA-A*02:01: | rUrA rArUrU rUrCrU rArCrU |
| | (TTTC)CCAGAGCC | rCrUrU rGrUrA rGrArU rCrCrA |
| | GTCTTCCCAGCCCA | rGrArG rCrCrG rUrCrU rUrCrC |
| | CC | rCrArG rCrCrC rArCrC |

TABLE 12
Knockdown efficiency of AsCas12a and GIG17
nucleases at the human B2M locus in Jurkat cells.

| | Knockdown efficiency (% HLA-A,B,C⁻ cells) | |
|---|---|---|
| sgRNA | AsCas12a | Gig17 |
| GR-44 | 0.43% | 1.63% |
| GR-45 | 10.90% | 1.88% |
| GR-46 | 0.61% | 4.97% |

TABLE 13
Knockdown efficiency of AsCas12a and GIG17 nucleases
at the human HLA-A*02:01 locus in T2 cells.

| | Knockdown efficiency (% HLA-A2⁻ cells) | |
|---|---|---|
| sgRNA | AsCas12a | Gig17 |
| GR-71 | 10.40% | 1.45% |
| GR-72 | 0.21% | 0.17% |
| GR-73 | 4.42% | 14.30% |

### 7. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### 8. EQUIVALENTS

While various specific embodiments have been illustrated and described, the above specification is not restrictive. It will be appreciated that various changes can be made without departing from the spirit and scope of the invention(s). Many variations will become apparent to those skilled in the art upon review of this specification.

### 9. SEQUENCES

TABLE 14
Sequences included in the Sequence Listing

| Sequence | Description |
|---|---|
| 1 | MAD7 nuclease sequence |
| 2-273 | MAD7 homolog sequences (predicted novel |
| | CRISPR nucleases) |
| 274-627 | Predicted CRISPR array repeat sequences |
| 628-631 | Nuclear localization sequences (NLSs) |
| 632-676 | E. coli codon optimized nucleotide sequence |
| 677-721 | E. coli codon optimized nucleotide sequence |
| | with 2x NLS (Nucloeplasmin & c-Myc), |
| | 6x His-tag, and stop codon |
| 722-766 | Human codon optimized nucleotide sequence |
| 767-811 | Human codon optimized nucleotide sequence |
| | with 2x NLS (Nucloeplasmin & c-Myc), |
| | 6x His-tag, and stop codon |
| 812 | pET21b-GIG17 Plasmid |
| 813 | pReceiver-EF1a-GIG17 Plasmid |
| 814 | T7p14-GIG17 Plasmid |
| 815-822 | Conserved sequences of cluster 1 |
| 823-832 | Conserved sequences of cluster 4 |
| 833 | Conserved sequence of cluster 6 |
| 834 | Conserved sequence of cluster 7 |
| 835-840 | Conserved sequences of cluster 9 |
| 841-844 | Conserved sequences of cluster 10 |

1-58. (canceled)

59. An engineered, non-naturally occurring targetable nuclease system comprising: (a) a nucleic acid-guided nuclease, comprising a nuclease polypeptide having at least 95% sequence identity to a sequence selected from SEQ ID NOs: 2-273, and (b) at least one engineered guide polynucleotide designed to form a complex with the nuclease and comprising a guide sequence, wherein the guide sequence is designed to hybridize with a target sequence in a eukaryotic cell, and (c) the complex of the nuclease and the guide polynucleotide do not naturally occur.

60. The targetable nuclease system of claim 59, wherein the nuclease polypeptide has at least 97% sequence identity to a sequence selected from SEQ ID NOs: 2-273.

61. The targetable nuclease system of claim 59, wherein the nuclease polypeptide has at least 100% sequence identity to a sequence selected from SEQ ID NOs: 2-273.

62. The targetable nuclease system of claim 59, wherein the nuclease polypeptide has at least 95% sequence identity to a sequence selected from SEQ ID NO: 2, 123, 116, 146, 43, 254, and 175.

63. The targetable nuclease system of claim 59, wherein the nuclease polypeptide has at least 97% sequence identity to a sequence of SEQ ID NO: 2.

64. The targetable nuclease system of claim 63, wherein the nuclease polypeptide has 100% sequence identity to a sequence of SEQ ID NO: 2.

65. The targetable nuclease system of claim 59, wherein the nuclease polypeptide has at least 95% sequence identity to a sequence selected from SEQ ID NOs.: 815-822.

66. The targetable nuclease system of claim 59, wherein the nuclease polypeptide has at least 95% sequence identity to a sequence selected from SEQ ID NO: 116 and 43.

67. The targetable nuclease system of claim 59, wherein the nuclease polypeptide is fused to a fusion peptide.

68. The targetable nuclease system of claim 67, wherein the fusion peptide is a signal peptide fused in-frame to the nuclease polypeptide.

69. The targetable nuclease system of claim 67, wherein the fusion peptide is a nuclear localization sequence fused to the nuclease polypeptide.

70. The targetable nuclease system of claim 69, wherein the nuclear localization sequence has a sequence selected from SEQ ID NO: 628-631.

71. The targetable nuclease system of claim 59, wherein the nuclease polypeptide is originated from Acidaminococcus massiliensis, Acidaminococcus sp., Acinetobacter indicus, Agathobacter rectalis, Anaerovibrio lipolyticus, Bacteroidales bacterium, Bacteroides galacturonicus, Bacteroides plebeius, Bacteroidetes bacterium, Butyrivibrio fibrisolvens, Butyrivibrio hungatei, Butyrivibrio sp., Candidatus Falkowbacteria bacterium, Candidatus Falkowbacteria bacterium, Candidatus Gottesmanbacteria bacterium, Candidatus Jacksonbacteria bacterium, Candidatus Magasanikbacteria bacterium, Candidatus Moranbacteria bacterium, Candidatus Pacebacteria bacterium, Candidatus Roizmanbacteria bacterium, Candidatus Ryanbacteria bacterium, Candidatus Saccharibacteria bacterium, Candidatus Sungbacteria bacterium, Candidatus Uhrbacteria bacterium, Candidatus Wildermuthbacteria bacterium, Candidatus Yonathbacteria bacterium, Catenovulum sp., Clostridiales bacterium, Clostridium sp., Coprococcus eutactus, Coprococcus sp., Deltaproteobacteria bacterium, Elizabethkingia sp., Eubacteriaceae bacterium, Eubacterium eligens, Eubacterium rectale, Eubacterium sp., Eubacterium ventriosum, Fibrobacter sp., Fibrobacter succinogenes, Firmicutes bacterium, Flavobacterium branchiophilum, Francisella hispaniensis, Francisella novicida, Francisella philomiragia, Francisella tularensis, Lachnospiraceae bacterium, Lachnospira pectinoschiza, Lentisphaeria bacterium, Leptospiraceae bacterium, Leptospira sp., Moraxella bovis, Moraxella bovoculi, Moraxella lacunata, Moraxella ovis, Moraxella sp., Muribaculaceae bacterium, Patescibacteria group bacterium, Phycisphaerae bacterium, Phycisphaerales bacterium, Porphyromonadaceae bacterium, Porphyromonas crevioricanis, Prevotella brevis, Prevotellaceae bacterium, Prevotella copri, Prevotellamassilia sp., Prevotella ruminicola, Prevotella sp., Prolixibacteraceae bacterium, Pseudobutyrivibrio sp., Pseudobutyrivibrio xylanivorans, Psychrobacter sp., Ruminococcaceae bacterium, Ruminococcus sp., Sedimentisphaera cyanobacteriorum, Sneathia amnii, Spirochaetia bacterium, Succinivibrionaceae bacterium, or Treponema sp.

72. A polynucleotide comprising a first polynucleotide segment encoding the nucleic acid-guided nuclease having at least 95% sequence identity to a sequence selected from SEQ ID NOs: 2-273.

73. The polynucleotide of claim 72, further comprising a second polynucleotide segment encoding a fusion peptide.

74. A vector encoding the nucleic acid-guided nuclease, comprising the polynucleotide of claim 72.

75. A host cell comprising the polynucleotide of claim 74.

76. A method of generating a nucleic acid-guided nuclease comprising the steps of:
culturing the host cell of claim 75, and
isolating the nucleic acid-guided nuclease from the host cell culture.

77. A method of modifying a target region of a eukaryotic or prokaryotic genome, comprising the steps of:
a. contacting a sample comprising the target region with
i. a nucleic acid-guided nuclease having at least 90% sequence identity to a sequence selected from SEQ ID NOs: 2-273, and
ii. a guide nucleic acid complexed with the nucleic acid-guided nuclease, and
b. allowing the nucleic acid-guided nuclease to modify the target region.
